(19) United States
(12) Patent Application Publication
(10) Pub. No.: US 2026/0210963 A1
(43) Pub. Date: Jul. 23, 2026

(54) SENSOR FOR DETECTING BIOMARKERS IN A FLUID SAMPLE AND METHODS OF USE

(71) Applicants: Arizona Board of Regents on behalf of Arizona State University, Scottsdale, AZ (US); American University of Beirut, Beirut (LB)

(72) Inventors: Jitendran Muthuswamy, Scottsdale, AZ (US); Massoud Khraiche, Beirut (LB)

(21) Appl. No.: 19/568,369

(22) Filed: Mar. 16, 2026

Related U.S. Application Data
(63) Continuation of application No. 18/566,986, filed on Jun. 6, 2022, now Pat. No. 12,578,334.
(60) Provisional application No. 63/196,814, filed on Jun. 4, 2021.

(51) Int. Cl. G01N 33/569; G01N 27/12; G01N 33/543

(52) U.S. Cl. CPC G01N 33/56983; G01N 27/12; G01N 33/5438; G01N 2333/165; G01N 2469/20

(57) ABSTRACT

The invention relates to a diagnostic platform for detection of biomarkers associated with a particular condition, disease, or disorder and methods of making and using the same. In various embodiments, the diagnostic platform includes a sensing device and an electronic reading platform.

### CROSS-REFERENCE TO RELATED APPLICATIONS

This application is continuation application of U.S. non-provisional patent application Ser. No. 18/566,986, filed on Dec. 4, 2023, which is a U.S. national stage entry under 35 U.S.C. § 371 of an International Application No. PCT/US2022/032379, filed on Jun. 6, 2022, which claims priority from U.S. provisional patent application No. 63/196,814, filed on Jun. 4, 2021, the contents of each of which are incorporated herein by reference in their entireties

### STATEMENT OF GOVERNMENT SPONSORSHIP

N/A

### REFERENCE TO A SEQUENCE LISTING

This application contains a Sequence Listing in computer readable form. The computer readable form is incorporated herein by reference. Said ASCII copy, created on Dec. 5, 2025, is named 2952332-000005-US2_SL.txt and is 248,295 bytes in size.

### FIELD OF THE INVENTION

The invention relates to a sensor device for detection of biomarkers associated with a particular condition, disease, or disorder and methods of making and using the same. The invention further includes a diagnostic platform that employs the sensor device.

### BACKGROUND OF THE INVENTION

Severe acute respiratory syndrome coronavirus 2 (SARS-COV-2) leads to the infectious disease COVID-19, which was first reported in Wuhan, China in December 2019. The disease has since spread across the globe infecting over 200 countries. The lack of cheap, scalable, and rapid testing platform has contributed significantly to the spread of the diseases as countries struggle to identify patients and isolate them to prevent the wide spread of the disease before health care systems are overwhelmed. The problem is exacerbated by the presence of many asymptomatic infected patients. In the absence of proven antiviral drug therapies and vaccines, the current pandemic containment and mitigation strategy depends on isolation of the infected individuals and their close contacts in addition to social distancing through large scale lockdowns for the entire countries or communities. The latter has strained economies across the world, currently risks the availability of resources and has paralyzed the world in ways that will take years to recover from [5]. However, to mitigate the risk of having resurge in cases, increasing testing capacity and access is fundamental for the rapid identification and isolation of COVID-19 cases and containment of any new clusters.

### SUMMARY OF THE INVENTION

Aspects of the invention are directed to a diagnostic platform for detection of at least one biomarker in a fluid sample. In embodiments, the diagnostic platform comprises a sensing device configured to receive the fluid sample. The platform can further comprise an electronic reading platform and a computing device. The electronic reading platform can be configured to activate the sensor. In embodiments, the sensing device comprises a layer of piezoelectric material comprising two faces; at least one electrode layer, wherein the at least one electrode layer is affixed to one face of the piezoelectric material; a second reference electrode layer that is affixed to a second face of the piezoelectric material; and a sensing layer disposed upon the electrode layer. In certain embodiments, the sensing layer is configured to bind the at least one biomarker for a disease or condition. The sensing device can be communicatively linked with the electronic reading platform, and the electronic reading platform can be configured to receive sensor data from the sensing device and to communicate the data to the computing device. The computing device is configured to determine the presence, absence, or amount of the at least one biomarker. The computing device can be integral with the electronic reading platform or external to the electronic reading platform.

In embodiments, the fluid sample comprises blood, saliva, nasal fluid, or a combination thereof. Certain embodiments comprise a fingerstick system configured to obtain the blood from a patient. The fingerstick system can be a belt-driven fingerstick system.

The diagnostic platform comprises a portable, hand-held device. In embodiments, the portable, hand-held device is configured to be worn by a user. The diagnostic platform configured to determine the presence, absence, or amount of the at least one biomarker within about 10 minutes after the fluid sample contacts the sensing device.

In certain embodiments, the piezoelectric material comprises a quartz crystal, PZT (lead zirconate titanate), lead titanate, Barium titanate, Zinc Oxide, lead magnesium niobate lead titanate (PMNPT), polyvinylidene difluoride, polyvinylidene fluoride (PVDF), Aluminum nitride, Gallium nitride, or a combination thereof.

The piezoelectric material can comprise a diameter of at least about 1 mm. In embodiments, the piezoelectric material comprises a diameter of up to about 153 mm.

In certain embodiments, the piezoelectric material comprises a thickness of at least about 10 μm. The piezoelectric material can comprise a thickness of up to about 3 mm.

In embodiments, the electrode layer comprises at least one working electrode. The at least one working electrode comprises a conductive film. The conductive film comprises gold, indium tin oxide (ITO), or a combination thereof.

In certain embodiments, the at least one working electrode is greater than about 1 nm thick. The at least one working electrode comprise a thickness of between about 1 nm and 500 nm, inclusive. The at least one working electrode can comprise a diameter of at least 10 μm. In embodiments, the at least one working electrode comprises a diameter of up to 150 mm.

In certain embodiments, the sensing device comprises a thickness shear mode (TSM) transducer. The sensing device can comprise a disposable sensor cartridge. In embodiments, the sensing layer comprises an antigen or an antibody that is specific for the at least one biomarker.

In embodiments, the disease or condition is caused by a coronavirus. The disease or condition can comprise COVID-19, severe acute respiratory syndrome, middle-east respiratory syndrome (MERS), a tissue inflammation or a combination thereof. In certain embodiments, at least one biomarker comprises IgG antibodies, IgM antibodies, or a combination thereof.

The sensing device can comprise at least two disposable sensor cartridges. In embodiments, one of the at least two sensor cartridges comprises a means for detecting IgG antibodies in the fluid sample, and the other sensor cartridge comprises a means for detecting IgM antibodies in the fluid sample.

In embodiments, the sensing layer comprises a severe acute respiratory syndrome coronavirus 2 (SARS-COV-2) structural protein or an antibody thereto immobilized on a surface of the at least one electrode layer, and IgG, IgM, or a combination thereof, wherein the IgG, IgM, or a combination thereof is tethered to the SARS-COV-2 structural protein. The SARS-CoV-2 structural protein or the antibody thereto can be immobilized to the surface of the at least one electrode layer via a self-assembled monolayer (SAM). The SAM can comprise a streptavidin-biotin bond, a thiol-bond, or a combination thereof.

In certain embodiments, the SARS-COV-2 structural protein comprises an S Protein, an N protein, an M protein, or a combination thereof.

In embodiments, the diagnostic platform can comprise at least one stabilizing solution that is configured to extend the shelf life of the diagnostic platform for up to at least 12 months.

The sensing device can be communicatively linked with the electronic reading platform via a USB connection. In embodiments, the computing device comprises a mobile computing device. In one embodiment, the diagnostic platform further comprises an application running on a processor of the mobile computing device, wherein the electronic reading platform is communicatively linked to the mobile computing device, and the electronic reading platform is configured to transmit sensor data to the mobile computing device. The electronic reading platform can be communicatively linked to the mobile computing device through one or more wireless communications protocols. The mobile computing device can comprise a portable digital assistant, a tablet, a smartphone, a laptop, or a combination thereof.

In another aspect, the invention comprises a method of predicting the existence or progression of a disease or condition or level of immunity in a patient. The method can comprise obtaining a fluid sample from the patient; placing the fluid sample on any of the various sensing device embodiments disclosed herein; permitting the diagnostic platform to determine the presence, absence, or amount of the at least one biomarker for the disease or condition; permitting the diagnostic platform to generate a sensor data report; reviewing the sensor data report; and predicting the presence or progression of the disease or condition. In embodiments, the method comprises two biomarkers, wherein the disease or condition comprises COVID-19, MERS, SARS, or a combination thereof; one of the two biomarkers comprises IgM antibodies and the remaining biomarker comprises IgG antibodies. In certain embodiments, the presence of IgM antibodies but not IgG antibodies indicates that the patient is in an intermediate stage of infection. The presence of IgG antibodies but not IgM antibodies can indicate that either the patient is in a late stage or an early stage of recurring infection or the patient is in a convalescent stage of infection. The presence of both IgM antibodies and IgG antibodies can indicate that either the patient is in a late phase of the infection, or the patient is in recovery stage of infection. In embodiments, the absence of IgG and IgM antibodies indicates that the patient does not have COVID-19.

In certain embodiments, the disease or condition comprises COVID-19, MERS, SARS, or a combination thereof. A first biomarker can comprise an S protein, a second biomarker can comprise an N protein, and a third biomarker can comprise an M protein, and the presence of any one or more of the biomarkers indicates the presence of the disease or condition.

### BRIEF DESCRIPTION OF THE DRAWINGS

The patent or application file contains at least one drawing executed in color. Copies of this patent or patent application publication with color drawings will be provided by the Office upon request and payment of necessary fee.

FIG. 1 shows a graphical representation of data collected from a literature survey showing the timeline of the COVID-19 with respect to the level of different known COVID-19 biomarkers.

FIG. 2 shows the presently disclosed system under one embodiment. FIG. 2A shows an exploded version of the system under one embodiment. FIG. 2B provides a detailed view of a sensor cartridge under one embodiment. FIG. 2C provides a top view of the full system in this embodiment. As shown in FIG. 2D, the system can be integrated with a software application on a portable device. FIG. 2E provides an illustration of a wearable embodiment of the presently disclosed.

FIG. 3 panel A shows fabricated TSM sensors under one embodiment of the present invention. A US penny is shown next to the TSM sensor for scale. FIG. 3 panel B provides a close-up view of a micro TSM under an embodiment of the present invention. FIG. 3 panel C a graphical plot showing regions under each curve combinations of electrode thicknesses and diameters that will suppress inharmonic modes of vibration. FIG. 3 panel D provides a schematic example of various steps involved in immobilizing GABA on the surface for anti-GABA sensing using TSM. This shows experience four group in anti-body sensing and immobilization chemistry FIG. 3 panel E provides a calibration curve for detection of anti-GABA antibody in PBS buffer (chemistry in FIG. 3 panel D). Maximum sensitivity of sensor to anti-GABA antibody is determined as the slope (red line) at the midpoint. The sensing interface consists of SAM/dextran/GABA on a gold electrode. [2,3]. The curve in FIG. 3 panel E is be fitted to the equation: Δf=Δf0+Δfmax/(1+Keq/C), where C is concentration, Keq equilibrium association constant [2].

FIG. 4 panel A provides Electrochemical Impedance Spectroscopy (EIS) data that show impact of poly(3,4-ethylenedioxythiophene) polystyrene sulfonate (PEDOT:PSS) multiple layer printing on 50 μm electrode impedance. FIG. 4 panel B-EIS shows impact of PEDOT:PSS electrode diameter on electrode impedance and phase angle. FIG. 4 panel C provides a close-up view of 16 Ag/PEDOT:PSS electrode leads printed with 50 μm line width on Polycaprolactone (PCL) under one embodiment (Left). A close-up view of recording sites where AgNPs leads terminate with PEDOT: PSS electrodes of 50 μm (Right). In this embodiment, the leads are passivated with PCL (Right). A 16-electrode neural interface printed on Polyimide (PI). Closeup of AgNPs lead terminate with graphene/PEDOT:PSS and PEDOT: PSS-coated electrodes of 50 μm. The leads are passivated with PVPh (Middle). FIG. 4 panel D provides a schematic showing a fabrication schematic that can be utilized when employing inkjet devices.

FIG. 5 provides a schematic workflow of the sensor device and associated diagnostic test under one embodiment. As shown, the system can be configured to diagnose COVID-19 according to the presence of IgM (top), IgG (bottom), or a combination thereof.

FIG. 6 shows the response of a miniaturized point-of-care, in vitro diagnostic sensor to different concentrations of monoclonal antibody to the nucleocapsid protein of the SARS-COV-2 virus, under one embodiment. The early sensitivity assessment was performed without any optimization of the resonator or electrode dimensions. The monoclonal antibodies were in a solution of bovine serum albumin and added to the sensor surface that had 60 μl of phosphate buffered saline solution.

FIG. 7 shows a reaction scheme of embodiments of immobilization chemistry to sense antibodies to Spike-protein of the SARS-COV-2 virus.

FIG. 8 shows a reaction scheme of embodiments of immobilization chemistry to sense antibodies to Spike-protein of the SARS-COV-2 virus.

FIG. 9 shows a reaction scheme of embodiments of immobilization chemistry to sense antibodies to Spike-protein of the SARS-COV-2 virus.

FIG. 10 shows a graph of QCM sensor data. In PBS, using 100% 11-MUA as the initial alkanethiol layer (SAM), the dynamic range of the sensor was ~0-0.8 μg/ml.

FIG. 11 shows a graph of QCM sensor data. In PBS, using 50% 11-MUA and 50% 9-Mercapto-1-nonanol as the initial alkanethiol layer (SAM), the dynamic range of the sensor increased to ~0-100 μg/ml.

FIG. 12 shows a graph of QCM sensor data. Testing sensor with spike antibody in calf blood plasma diluted in PBS.

FIG. 13 shows a graph of QCM sensor data. QCM sensor was responsive to [Spike Ab] in plasma when diluted in PBS. Results of sensitivity analysis show the sensor demonstrating a linear response to increasing concentrations of anti-S IgG in plasma from calf-blood. (Inset) Time series responses of the sensor (change in frequency) to 4 different concentrations of anti-S IgG in plasma from calf blood. A response of ~48.3 Hz at 0 ng/ml indicates the response of the Acousto-Ab sensor to non-specific binding of plasma proteins in calf blood.

FIG. 14 shows a photograph of a two channel quartz resonators fabricated on a glass substrate. Scale bar=300 mm. Data published in Khraiche and Muthuswamy, Lab on Chip, 2012, 12, 2930-2941.

FIG. 15 shows a photograph of an example of fluid spotting precision on 30 μm diameter electrodes.

FIG. 16 shows an illustration of blood being sampled by a lancet pen and dispensed into a dilution well (filled with PBS). It is then filtered using a graphene filter before releasing plasma into the sensor well in the cartridge. Illustration of blood being sampled by a lancet pen and dispensed into a dilution well. It is then filtered using a graphene filter before releasing plasma into the sensor well in the cartridge.

FIG. 17 provides an illustration of the overall concept of Acousto-Ab systems for sensing antibodies in blood. (Panel A) The user will use a lancet to draw a drop of blood. (Panel B) the blood sample will be added to the sensor well in a cartridge (inset) that is integrated to an electronic reader. (Panel C) Multiple sensing electrodes defined on a resonating platform will enable simultaneous detection of multiple antibodies to proteins in the blood sample. (Panel D) Changes in resonant frequency of the resonator under a given sensing electrode is directly proportional to the concentration of the target antibody and (Panel E) will be transmitted wirelessly by the reader to an external hand-held device.

FIG. 18 provides an illustration of the surface immobilization chemistry and the operating principle of the Acousto-Ab system—(Panel A) biotynlated Spike protein are bound to a self-assembled monolayer terminating in streptavidin. Any IgG captured from serum or plasma sample is detected by the subsequent addition of anti-IgG. (Panel B) similar scheme for the detection of IgM. (Panel C) & (Panel D). Molecular adhesion to the gold electrodes on the quartz resonator causes a decrease in the resonant frequency of the resonator, in direct proportion to the mass of adhered molecules. We will use surface immobilization chemistry for Nucleocapsid (N-protein) and NSP5 proteins.

### DETAILED DESCRIPTION OF THE INVENTION

The present invention relates to a sensor device for detection of biomarkers associated with a particular condition, disease, or disorder and methods of making and using the same. In embodiments, the invention comprises a highly sensitive diagnostic platform for rapid detection of seroprevalence of a disease or condition is caused by a coronavirus. The disease or condition can comprise COVID-19, severe acute respiratory syndrome, middle-east respiratory syndrome (MERS), a tissue inflammation or a combination thereof. The diagnostic platform can be configured to permit rapid detection of antigens of SARS-COV-2.

Before the present disclosure is described in greater detail, it is to be understood that this disclosure is not limited to particular embodiments described, and as such can, of course, vary. It is also to be understood that the terminology used herein is for the purpose of describing particular embodiments only, and is not intended to be limiting.

The discussion of the background to the invention herein is included to explain the context of the present invention. This is not to be taken as an admission that any of the material referred to was published, known, or part of the common general knowledge in any country as of the priority date of any of the claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges can independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein can have the same meaning as commonly understood by one of ordinary skill in the art to which this disclosure belongs. Any methods and materials similar or equivalent to those described herein can also be used in the practice or testing of the present disclosure.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that can need to be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, nanotechnology, organic chemistry, biochemistry, botany and the like, which are within the skill of the art. Such techniques are explained fully in the literature.

### Abbreviations and Definitions

Detailed descriptions of one or more embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it can modify that range by extending the boundaries above and below the numerical values set forth. In general, the term “about” is used herein to modify a numerical value above and below the stated value by a variance of 20 percent up or down (higher or lower).

The terms “sufficient” and “effective”, as used interchangeably herein, can refer to an amount (e.g. mass, volume, dosage, concentration, and/or time period) needed to achieve one or more desired result(s).

The term “administration” can refer to introducing a composition of the present disclosure into a subject. For example, one route of administration of the composition is intracranial administration. As another example, the composition can be administered by intravenous administration. However, any route of administration, such as topical, subcutaneous, peritoneal, intraarterial, inhalation, vaginal, rectal, nasal, introduction into the cerebrospinal fluid, or instillation into body compartments can be used.

As used herein, “treat”, “treatment”, and/or “treating” can refer to acting upon a condition (e.g., inflammation), a disease or a disorder with a composition to affect the condition (e.g., inflammation), disease or disorder by improving or altering it. The improvement or alteration can include an improvement in symptoms or an alteration in the physiologic pathways associated with the condition (e.g., inflammation), disease, or disorder. “Treatment” can refer to one or more treatments of the disease or condition in a subject (e.g., a mammal, typically a human or non-human animal of veterinary interest), and can include: (a) reducing the risk of occurrence in a subject determined to be predisposed to the condition or disease but not yet diagnosed with it (b) impeding the development of the condition or disease, and/or (c) relieving the condition or disease, e.g., causing regression of the condition or disease and/or relieving one or more condition or disease symptoms. As used herein, the terms “prophylactically treat” or “prophylactically treating” can refer to completely or partially preventing (e.g., about 50% or more, about 60% or more, about 70% or more, about 80% or more, about 90% or more, about 95% or more, or about 99% or more) a condition (e.g., condition or disease), a disease, or a symptom thereof and/or can be therapeutic in terms of a partial or complete cure for a condition (e.g., condition or disease), a disease, and/or adverse effect attributable to the disease.

As used herein, “therapeutic” can refer to curing or treating a symptom of a disease or condition.

As used herein, the term “subject,” or “patient,” can include humans and mammals (e.g., mice, rats, pigs, cats, dogs, and horses), and non-mammals (e.g., aves such as chickens etc.). Typical subjects to which compounds of the present disclosure can be administered will be mammals, particularly primates, especially humans. For veterinary applications, a wide variety of subjects will be suitable, non-limiting examples of which comprise livestock such as cattle, sheep, goats, cows, swine; poultry such as chickens, ducks, geese, turkeys; and domesticated animals particularly pets such as dogs and cats. For diagnostic or research applications, a wide variety of mammals can be suitable subjects, non-limiting examples of which comprise rodents (e.g., mice, rats, hamsters), rabbits, primates, and swine such as inbred pigs and the like. The term “living subject” can refer to a subject noted above or another organism that is alive.

As used herein, the term “fluid sample” can refer to a body fluid sample including, without limitation blood, plasma, cerebrospinal fluid, and other body fluids. The fluid sample can comprise a serologic sample. The body fluid sample can be diluted with, e.g., buffer or other reagents that facilitate handling. As used herein, the term “vapor sample” is intended to mean a sample containing a non-liquid component and optionally entrained liquid component. A preferred vapor sample is exhaled breath, which can be diluted with additional gas prior to detection or concentrated by removing certain components of the vapor sample. Both fluid samples and vapor samples can be used to detect the drug or metabolite concentration. As used herein, the term “sample” without further description is intended to encompass both fluid samples and vapor samples.

Before explaining at least one embodiment of the disclosure in detail, it is to be understood that the disclosure is not necessarily limited in its application to the details set forth in the following description or exemplified by the examples. The disclosure is capable of other embodiments or of being practiced or carried out in various ways. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Accordingly, a first aspect of the present invention relates to a diagnostic platform for the detection of at least one biomarker in a fluid sample. In embodiments, the diagnostic platform comprises a sensing device and an electronic reading platform. Under certain embodiments, the sensing device comprises a layer of piezoelectric material, at least one electrode layer, a sensing layer, or a combination thereof. In embodiments, the sensing layer is disposed upon the electrode layer and is configured to bind at least one biomarker of a particular disease or condition.

In one embodiment, the diagnostic platform is configured to test for the serological prevalence of COVID-19.

### Sensors

In various embodiments, the sensing device disclosed herein comprises an acoustic sensor configured to detect the presence of certain biomarkers of a disease or condition. In embodiments, the sensor comprises a piezoelectric material that operates as a transducer of a detection event. The piezoelectric material can comprise quartz crystals or any piezoelectric material known in the art. Exemplary piezoelectric material includes, but is not limited to, Sucrose (table sugar), Rochelle salt, Topaz, Tourmaline, Berlinite (AlPO₄), Barium titanate (BaTiO₃), Lead titanate (PbTiO₃), Lead zirconate titanate (PZT), Piezoelectric ceramic, Potassium niobate (KNbO₃), Lithium niobate (LiNbO₃), Lithium tantalate (LiTaO₃), Sodium tungstate (Na₂WO₄), Sodium potassium niobate (NaKNb), Bismuth ferrite (BiFeO₃), Sodium niobate (NaNbO₃), Collagen, Gallium orthophosphate (GaPO₄), Langasite (La₃Ga₄SiO₁₄), lead magnesium titanate-lead titanate (PMNPT), zinc Oxide, aluminum nitride (AlN), polyvinylidene difluoride, polyvinylidene fluoride (PVDF), or a combination thereof,.

In certain embodiments, AT-cut quartz produces bulk transverse shear waves with particle displacements parallel to the surface of the crystal and its electrodes. The application of an electric field across the thickness of AT-cut quartz can lead to particle movement, which in turn results in two types of standing waves—a transverse wave in the thickness direction referred to as the thickness shear wave TS₁ and a wave traveling in the radial direction known as the thickness twist TT₃ wave. In one embodiment, the path length of TS₁ waves is the plate thickness with nodes along the diameter of the plate, while the path length for TT₃ is the electrode radius with concentric nodal lines along the center of the plate. When the length of the path is an integral number of wavelengths, a standing wave occurs and results in resonance [20]. The fundamental resonant frequency of AT-cut quartz is a result of the TS₁ standing wave and is a reliable and large wave of this type of acoustic systems. These AT-cut quartz oscillators can also be called thickness shear mode (TSM) resonator given the displacement direction. In operation, when a small mass, such as a biomarker for a particular disease or condition is deposited on the surface of a quartz crystal oscillator, the oscillator's resonance frequency decreases in direct proportion to the deposited mass as described by the classic Sauerbrey equation for sensitivity of the resonator, which is provided below as Equation 1:

$$Δ {f}_{o} = \frac{2 {f}_{o}^{2}}{{( {ρ}_{Q} {μ}_{Q} )}^{1 / 2}} \frac{Δ m}{A}$$

Wherein fₒ is the fundamental resonant frequency of the quartz crystal, A is the surface area of the electrode on top of the crystal, μ_{Q} and ρ_{Q} are the shear modulus and the density of quartz. While m is the mass deposited on sensor.

As noted herein, the acoustic sensor is intended to be in contact with a fluid sample. As such, during use, the electrochemical sensor is intended to be exposed to a fluid sample. To facilitate exposure to the fluid sample, a fluid sample can be drawn from the patient and then exposed ex vivo to the sensor or sensing device. The sensor or sensor device according to any embodiment described herein is suitable for ex vivo detection of a biomarker for a particular disease or condition.

As discussed herein, the sensing device can comprise a sensing layer configured to bind at least one biomarker for a disease, infection, or condition. In embodiments, the sensing layer comprises a probe for detection of a biomarker, wherein the probe is immobilized, fixed, anchored, or otherwise tethered to a solid support. The probe can comprise a polypeptide, a polynucleotide, or a combination thereof. For example, in embodiments, the probe can be an antibody, an antibody fragment, an antigen (such as a portion of a viral spike protein or cell wall protein), or a fragment of antigen. The term “antibody fragment”, as used herein, can be a portion of an antibody such as F_{(ab′)2}, F_{(ab)2}, F_{ab}′, F_{ab}, Fv, scFv and the like. Non-limiting examples of antibody fragments that can be attached to a support include Fab, Fab′ and F(ab′)₂, Fd, Fvs, single-chain Fvs (scFv), single-chain antibodies, dAb (domain antibody), minibodies, disulfide-linked Fvs (sdFv), fragments comprising either a VL or VH domain, fragments produced by a Fab expression library, and anti-idiotypic (anti-Id) antibodies.

The probe can be covalently or non-covalently attached to the support. In some embodiments, the probe (such as the antibody, antigen, or fragment thereof) can be cross-linked to the solid support for immobilization. The skilled artisan understands it can use a crosslinker to aim at the primary amine (—NH₂) and carboxyl (—COOH) groups since they are abundant and well distributed over the protein surface. Non-limiting examples of cross-linkers include NHS esters, imidoesters, or glutaraldehyde for amine-to-amine conjugation, carbodiimide for carboxyl-to-amine linking, maleimide or epoxide for sulfhydryl groups, and hydrazides for aldehyde groups (see also, Shen at al., Methods. 2017 Mar. 1; 116:95-111; which is incorporated by reference in its entirety). In some embodiments, the solid support can comprise one or more amine(s), hydroxyl(s) or epoxide(s) to immobilize the probe. For example, the probe can be an antibody or antibody fragment that is attached to the support. For example, the probe can be an antigen or antigen fragment that is attached to the support. In some embodiments, the support can be porous or non-porous. Examples of non-porous supports include polystyrene, polyethylene, dextran, polypropylene, plastic, and glass. In some embodiments, the support can be transparent. The probe can be tethered to the solid support via a sandwich assay. In embodiments, the probe is tethered to an amino acid sequence or a polynucleotide sequence that is immobilized the support surface. In one embodiment, the sandwich assay comprises immobilized S protein on the surface and ant-IgM and IgG in buffer topping the sensor to detect IgM and IgG (as illustrated in FIG. 5—panels 2a & 2b). In embodiments, the probe is immobilized utilizing a conjugation of biotin and streptavidin, biotin and avidin, a sugar and lectin, or a combination thereof. One embodiment comprises amination of the electrode surface via self-assembled mono-layer (SAM) of 11-amino-1-undecanethiol or 9-amino-1-undecanethiol. This can be followed by covalent attachment of streptavidin and then biotinylated S protein are attached via streptavidin-biotin bond. Such a process of electrode surface amination followed by a streptavidin-biotin bridge can be utilized to attach any protein to the sensor surface. Another embodiment comprises amination of the electrode surface via SAM of 11-amino-1-undecanethiol or 9-amino-1-undecanethiol. This can be followed by covalent attachment of antibodies to S-protein or N-protein or M-protein or E protein.

Wherein the diameter of the sensing electrode (non-liming examples include about 10 μm-about 15 mm) is optimized along with the thickness of the sensing layer above the electrode (non-limiting examples include about 30-about 150 nm), and the operating resonant frequency (e.g. about 1-about 100 MHz) of the piezoelectric substrate for a given viscosity (e.g. about 0.1-about 10 cP) of the sample fluid (blood, saliva, nasal swab or other biofluid) to achieve the desired Q-factor (e.g. about 100-about 100,000), sensitivity (e.g. about 1 pg/ml-about 100 μg/ml) and detection limits. Additionally, for a given operating resonant frequency, the diameter and thickness of the sensing electrode (e.g. about 1-about 500 nm) can be optimized to maximize energy trapping and mitigate inharmonic modes of oscillation under fluid. The number of sensing electrodes (e.g. about 1-about 100,000) on the piezoelectric substrate is determined by the number of independent, simultaneous measurements that need to be performed. In embodiments, the thickness, diameter size, or both of the at least one working electrode can be configured to optimize energy trapping, mitigate inharmonic modes in the bulk piezoelectric with standing liquid (serum or buffer or saliva or any other sample fluid) on top of the working electrode.

### Biomarkers

As discussed herein, various embodiments of the disclosure permit the detection of biomarkers in a sample as an indicator of a particular disease, infection, or condition. In embodiments, biomarkers include any biological marker whose presence or absence is known to be associated with a particular disease, infection, or condition. Biomarkers can be useful for diagnosing a patient with a particular disease, infection, or condition; predicting clinical outcomes of a patient suffering from a disease, infection, or condition, directing individualized treatment decisions for a patient suffering from a particular disease, infection, or condition, predicting the likelihood that a particular treatment will be effective, assessing the effectiveness of a particular treatment response, or a combination thereof. Biomarkers can be specific for a particular disease, infection, or condition, showing little to no cross-over to alternate disease states.

In embodiments, biomarkers can comprise biological indicators of infectious disease caused by a microorganism (such as the presence of antigens or antibodies). In some embodiments, the infectious disease can be caused by a microorganism, such as a DNA virus, RNA virus, or reverse transcribing virus. Non-limiting examples of viruses include Adenovirus, Coxsackievirus, Epstein-Barr virus, Hepatitis A virus, Hepatitis B virus, Hepatitis C virus, Herpes simplex virus, type 1, Herpes simplex virus, type 2, Cytomegalovirus, Human herpesvirus, type 8, HIV, Influenza virus, Measles virus, Mumps virus, Human papillomavirus, Parainfluenza virus, Poliovirus, Rabies virus, Respiratory syncytial virus, Rubella virus, Varicella-zoster virus. In embodiments, biomarkers can comprise biological indicators of a coronavirus, such as SARS-CoV-2 or COVID-19. Representative coronaviruses include but are not limited to human coronavirus NL63 (HCoV-NL63), porcine transmissible gastroenteritis coronavirus (TGEV), porcine epidemic diarrhea coronavirus (PEDV), and porcine respiratory coronavirus (PRCV) in the genus Alphacoronavirus; severe acute respiratory syndrome coronavirus (SARS-COV), Middle East respiratory syndrome coronavirus (MERS-COV), bat coronavirus HKU4, mouse hepatitis coronavirus (MHV), bovine coronavirus (BCOV), and human coronavirus OC43 in the genus Betacoronavirus; avian infectious bronchitis coronavirus (IBV) in the genus Gammacoronavirus; and porcine deltacoronavirus (PdCV) in the genus Deltacoronavirus Embodiments can also include biomarkers for Influenza A, Influenza B, and its different sub-types, or for any other disease condition for which appropriate biomarkers can be found.

In some embodiments, the infectious disease can be caused by a microorganism, such as a Gram-positive bacterium, a Gram-negative bacterium, a protozoa, or a fungus. Non-limiting examples of disease-causing bacteria include: Bacillus anthracis, Bacillus cereus, Bartonella henselae, Bartonella Quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheria, Enterococcus faecalis, Enterococcus faecium, Escherichia coli, Francisella tularensis, Haemophilus influenza, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitides, Pseudomonas aeruginosa, Rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholera, Yersinia pestis, Yersinia enterocolitica, Yersinia pseudotuberculosis. Non-limiting examples of disease-causing protozoa include: Plasmodium falciparum (malaria), Toxoplasma gondii (toxoplasmosis), Leishmania species (leishmaniases), Trypanosoma brucei (Δfrican sleeping sickness), Trypanosoma cruzi (Chagas disease), and Giardia intestinalis (giardiasis). Non-limiting examples of disease-causing fungi include Candida albicans, Aspergillus fumigatus, Aspergillus flavus, Cryptococcus neoformans, Cryptococcus gattii, Histoplasma capsulatum, Pneumocystis carinii, Stachybotrys chartarum.

For example, the biomarkers described herein can be found in saliva, nasal swabs, blood samples or other biological samples.

The SARS-COV-2 genome encodes for four major structural proteins: the spike (S, QHD43416.1), membrane (M, QHD43419.1), envelope (E, QHD43418.1) and nucleocapsid (N, QHD43423.2) proteins, each of which can serve as a biomarker for COVID-19. The S protein comprises 2 subunits, S1 and S2. S1 mediates the binding of the virus to the host cell receptor, while S2 contains other elements required for membrane fusion. The M protein is the most abundant structural protein that defines the shape of the virus. The N protein is the most abundantly shed viral protein during infection and can be detected in serum and urine samples within the first 2 weeks of infection. The smallest major structural protein, E protein, participates in viral assembly and pathogenesis.

Exemplary COVID-19 biomarkers include SARS-COV-2 RNA detected via envelope (E), RNA-dependent RNA polymerase (RdRp) genes (YP_009725307, RdRp/helicase (H) genes (YP_009725308). Additional COVID-19 biomarkers include nucleocapsid protein (N, QHD43423.2), and ORF1b (BCN86436.1), which are highly conserved among other respiratory viruses. An additional exemplary biomarker comprises ORFla. In some embodiments, the technology described herein can be useful for the detection of SARS-COV2 variants. For example, the variants can be: the UK variant B.1.1.7 (such as B.1.1.7 with S: E484K); the South Δfrican variant B.1.351; the California variant B.1.427; the California variant B.1.429; the Brazilian variant P.1; the Brazilian variant P.2; the New York variant B.1.526 (such as B.1.526 with S: E484K or B.1.526 with S: S477N); the New York variant B.1.526.1; the New York variant B.1.526.2, the amino acid mutations of each strain which can be accessed at https://outbreak.info/situation-reports #Lineage_Mutation, and is incorporated by reference in their entireties. For example, a variant of SARS-COV2 has accession number YP 009724390.1. For example, a variant of SARS-COV2 has accession number QHD43416.1.

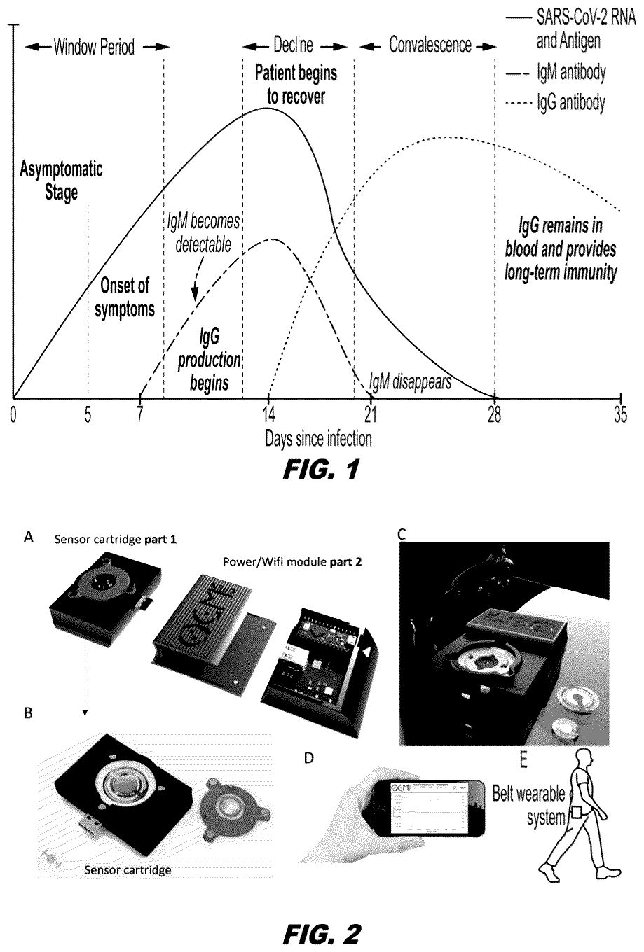

Antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, IgG₃, IgG₄. Furthermore, in humans, the light chain can be a kappa chain or a lambda chain. The term “antigen-binding site,” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In embodiments, additional biomarkers include antibodies for SARS-COV-2 that can be present in the serum or plasma of patients. In certain embodiments, such antibodies include IgG, IgM, IgA antibodies, or a combination thereof. IgG can be detectable starting 13 to 21 days after infection and persists for long durations. IgM response on the other hand occurs earlier, at around 10 days after infection, but then decreases rapidly after 35 days and disappears. Data in FIG. 1 is collected from a literature survey showing the timeline of the disease with respect to the level of biomarkers (citations to the data points are in the references cited in Example 1).

Biomarkers can include any isotopes, fragments, variants, derivatives, or other modifications of any of the foregoing.

Biomarkers can include any one or more of the following amino acid and nucleotide sequence.

The amino acid sequence of the spike protein(S) (Severe acute respiratory syndrome coronavirus 2; GenBank: QHD43416.1; SEQ ID NO: 1) is:

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGENCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

The amino acid sequence of the membrane (M) protein (Severe acute respiratory syndrome coronavirus 2; GeneBank: QHD43419.1; SEQ ID NO: 2) is:

MADSNGTITVEELKKLLEQWNLVIGFLFLTWICLLQFAYANRNRFLYIIK
LIFLWLLWPVTLACFVLAAVYRINWITGGIAIAMACLVGLMWLSYFIASF
RLFARTRSMWSFNPETNILLNVPLHGTILTRPLLESELVIGAVILRGHLR
IAGHHLGRCDIKDLPKEITVATSRTLSYYKLGASQRVAGDSGFAAYSRYR
IGNYKLNTDHSSSSDNIALLVQ

The amino acid sequence of the envelope (E) protein (Severe acute respiratory syndrome coronavirus 2; GeneBank Accession No. QHD43418.1; SEQ ID NO: 3) is:

MYSFVSEETGTLIVNSVLLFLAFVVFLLVTLAILTALRLCAYCCNIVNVS
LVKPSFYVYSRVKNLNSSRVPDLLV

The amino acid sequence of the nucleocapsid (N) protein (Severe acute respiratory syndrome coronavirus 2; GeneBank: QHD43423.2; SEQ ID NO: 4) is:

MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWVATEGALNTPKDHIGTRN
PANNAAIVLQLPQGTTLPKGFYAEGSRGGSQASSRSSSRSRNSSRNSTPG
SSRGTSPARMAGNGGDAALALLLLDRLNQLESKMSGKGQQQQGQTVTKKS
AAEASKKPRQKRTATKAYNVTQAFGRRGPEQTQGNFGDQELIRQGTDYKH
WPQIAQFAPSASAFFGMSRIGMEVTPSGTWLTYTGAIKLDDKDPNFKDQV
ILLNKHIDAYKTFPPTEPKKDKKKKADETQALPQRQKKQQTVILLPAADL
DDFSKQLQQSMSSADSTQA

The amino acid sequence of helicase (H) (Severe acute respiratory syndrome coronavirus 2; NCBI Reference Sequence: YP_009725308.1; SEQ ID NO: 5) is:

AVGACVLCNSQTSLRCGACIRRPFLCCKCCYDHVISTSHKLVLSVNPYVC
NAPGCDVTDVTQLYLGGMSYYCKSHKPPISFPLCANGQVFGLYKNTCVGS
DNVTDFNAIATCDWTNAGDYILANTCTERLKLFAAETLKATEETFKLSYG
IATVREVLSDRELHLSWEVGKPRPPLNRNYVFTGYRVTKNSKVQIGEYTF
EKGDYGDAVVYRGTTTYKLNVGDYFVLTSHTVMPLSAPTLVPQEHYVRIT
GLYPTLNISDEFSSNVANYQKVGMQKYSTLQGPPGTGKSHFAIGLALYYP
SARIVYTACSHAAVDALCEKALKYLPIDKCSRIIPARARVECFDKFKVNS
TLEQYVFCTVNALPETTADIVVFDEISMATNYDLSVVNARLRAKHYVYIG
DPAQLPAPRTLLTKGTLEPEYFNSVCRLMKTIGPDMFLGTCRRCPAEIVD
TVSALVYDNKLKAHKDKSAQCFKMFYKGVITHDVSSAINRPQIGVVREFL
TRNPAWRKAVFISPYNSQNAVASKILGLPTQTVDSSQGSEYDYVIFTQTT
ETAHSCNVNRFNVAITRAKVGILCIMSDRDLYDKLQFTSLEIPRRNVATL
Q

The amino acid sequence of RNA-Dependent RNA polymerase (RdRp) (Severe acute respiratory syndrome coronavirus 2; NCBI Reference Sequence: YP_009725307.1; SEQ ID NO: 6) is:

SADAQSFLNRVCGVSAARLTPCGTGTSTDVVYRAFDIYNDKVAGFAKFLK
TNCCRFQEKDEDDNLIDSYFVVKRHTFSNYQHEETIYNLLKDCPAVAKHD
FFKFRIDGDMVPHISRQRLTKYTMADLVYALRHFDEGNCDTLKEILVTYN
CCDDDYFNKKDWYDFVENPDILRVYANLGERVRQALLKTVQFCDAMRNAG
IVGVLTLDNQDLNGNWYDFGDFIQTTPGSGVPVVDSYYSLLMPILTLTRA
LTAESHVDTDLTKPYIKWDLLKYDFTEERLKLFDRYFKYWDQTYHPNCVN
CLDDRCILHCANFNVLFSTVFPPTSFGPLVRKIFVDGVPFVVSTGYHFRE
LGVVHNQDVNLHSSRLSFKELLVYAADPAMHAASGNLLLDKRTTCFSVAA
LTNNVAFQTVKPGNFNKDFYDFAVSKGFFKEGSSVELKHFFFAQDGNAAI
SDYDYYRYNLPTMCDIRQLLFVVEVVDKYFDCYDGGCINANQVIVNNLDK
SAGFPFNKWGKARLYYDSMSYEDQDALFAYTKRNVIPTITQMNLKYAISA
KNRARTVAGVSICSTMTNRQFHQKLLKSIAATRGATVVIGTSKFYGGWHN
MLKTVYSDVENPHLMGWDYPKCDRAMPNMLRIMASLVLARKHTTCCSLSH
RFYRLANECAQVLSEMVMCGGSLYVKPGGTSSGDATTAYANSVFNICQAV
TANVNALLSTDGNKIADKYVRNLQHRLYECLYRNRDVDTDFVNEFYAYLR
KHFSMMILSDDAVVCFNSTYASQGLVASIKNFKSVLYYQNNVFMSEAKCW
TETDLTKGPHEFCSQHTMLVKQGDDYVYLPYPDPSRILGAGCFVDDIVKT
DGTLMIERFVSLAIDAYPLTKHPNQEYADVFHLYLQYIRKLHDELTGHML
DMYSVMLINDNTSRYWEPEFYEAMYTPHTVLQ

The amino acid sequence of ORF1b (Severe acute respiratory syndrome coronavirus 2; NCBI Sequence: BCN86436.1; SEQ ID NO: 7) is:

# 5′3′ Frame 1
VQPVLHRAAQALVLMSYTGLLTSTMIK-LVLLNS-KLIVVASKKRTKMTI-LILTL-LRDTLSLTTNMKKQFIIYLR
IVQLLLNMTSLSLE-TVTWYHIYHVNVLLNTQWQTSSML-GILMKVIVTH-KKYLSHTIVVMMIISIKRTGMIL-KT
QIYYAYTPT-VNVYAKLC-KQYNSVMPCEMLVLLVY-H-IIKISMVTGMISVISYKPRQVVEFLL-ILIIHC-CLY-
P-PGL-LQSHMLTLT-QSLTLSGIC-NMTSRKRG-NSLTVILNIGIRHTTQIVLTVWMTDAFCIVQTLMFYSLQCSH
LQVLDH--EKYLLMVFHL-FQLDTTSES-VLYIIRM-TYIALDLVLRNYLCMLLTLLCTLLLVIYY-INALRAFQ-L
HLLTMLLFKLSNPVILTKTSMTLLCLRVSLRKEVLLN-NTSSLLRMVMLLSAIMTTIVIIYQQCVISDNYYL-LKLL
ISTLIVTMVAVLMLTKSSSTT-TNQLVFHLINGVRLDFIMIQ-VMRIKMHFSHIQNVMSSLL-LK-ILSMPLVQRIE
LAP-LVSLSVVL-PIDSFIKNY-NQ-PPLEELL--LEQANSMVVGTTC-KLFIVM-KTLTLWVGIILNVIEPCLTCL
ELWPHLFLLANIQRVVACHTVSID-LMSVLKY-VKWSCVAVHYMLNQVEPHQEMPQLLMLIVFLTFVKLSRPMLMHF
YLLMVTKLPISMSAIYNTDEMSVSIEIEMLTQTL-MSFTHICVNISQ--YSLTMLLCVSIALMHLKV-WLA-RTLSQ
FFIIKTMFLCLKQNVGLRLTLLKDLMNFALNIQC-LNRVMIMCTFLTQIHQES-GPAVL-MIS-KQMVHL-LNGSCL
-L-MLTHLLNILIRSMLMSFICTYNT-ESYMMS-QDTC-TCILLCLLMITLQGIGNLSFMRLCTHRIQSYRLLGLVF
FAIHRLH-DVVLAYVDHSYVVNAVTTMSYQHHIN-SCLLIRMFAMLQVVMSQM-LNFT-EV-AIIVNHINHPLVFHC
VLMDKFLVYIKIHVLVAIMLLTLMQLQHVTGQMLVITE-LTPVLKDSSFLQQKRSKLLRRHLNCLMVLLLYVKCCLT
ENYIFHGKLVNLDHHLTEIMSLLVIV-LKTVKYK-ESTPLKKVTMVMLLFTEVQQLTN-MLVIILC-HHIQ-CH-VH
LH-CHKSTMLELLAYTQHSISQMSFLAMLQIIKRLVCKSILHSRDHLVLVRVILLLA-LSTTLLLA-CIQLALMPLL
MHYVRRH-NICL-INVVELYLHVLV-SVLINSK-IQH-NSMSFVL-MHCLRRQQI-LSLMKFQWPQIMI-VLSMPDY
VLSTMCTLATLLNYLHHAHC-LRAH-NQNISIQCVDL-KL-VQTCSSELVGVVLLKLLTL-VLWFMIISLKHIKTNQ
LNALKCFIRVLSRMMFHLQLTGHK-AW-ENSLHVTLLGEKLSLFHLIIHRML-PQRFWDYQLKLLIHHRAQNMTMSY
SLKPLKQLTLVM-TDLMLLLPEQK-AYFA-CLIETFMTSCNLQVLKFHVGMWQLYKLKM-QDSLKIVVR-SLGYILH
RHLHTSVLTLNSKLKVYVLTYLAYLRT-PIEDSSL-WVLK-IIKLMVTLTCLSPAKKL-DMYVHGLASMSRGVMLLE
KLLVPIYLYS-VFLQVLT-LLYLQVMLIHLIIQIFPELVLNHRLEINLNTSYHLCTKDFLGM-CV-RLYKC-VTHLK
ISLTESYLSYGHMALS-HL-SIL-K-DLSAPVVYVIDVPHAFPLLQTLMPVGIILLDLITSIIRL-LMENNGVLQVT
YKATMICIVKSMVMHM-LVVMQS-LGV-LSTSALLSVLTGLLNIL-LVMN-RLMRLVERFNTWLLKLHY-QTNSQFF
TTLVTLKLLSVYLKLM-NGSSMMHSLVVTKLIK-KNYSILMPHILTNSQMVYAYFGIAMSIDILLIPLFVDLTLECY
LTLTCLVVMVAVCM-INMHSTHQLLIKVLLLI-NNYHFSITLTVHVSLMENK-CQI-IMYH-SLLRV-HVAI-VVLS
VDIMLMSTDCISMLIT--SQLALACGFTNNLILITSGTLLQDFRV-KMWLLML-IRDTLMDNRVKYQFLSLITLFTQ
KLMVLM-NCLKIKQHYLLM-HLSFGLSATLNQYQR-KYSIIWVWTLLLIL-SGTTKEMLQHIYLLLVFVL-LT-PRN
QLKRFVHHSLSFLMVELMVK-TYLEMPVMVFLLQKVVLKVYNHL-VPNKLVLMESH-LEKP-KHSSIIIRKLMVLSN
NYLKLTLLRVEIYKNLNPGVKWKLIS-N-LWMNSLNGIN-KAMPSNISFMEILVIVS-VVYIY-LD-LNVLRNHLLN
-KILFLWTVQLKTIS-QMRKQVHLSVCVLLLIYYLMILLK--NPKIYL-FLRLSK-LLTIQKFHLCFGVKMAM-KHF
TQNYNLVKRGNRVLLCLIFTKCKECY-KSVTFKIMVIVQHYLKA---MSQNILNCVNI-TH-H-LYPII-ELYILVL
VLIKELHQVQLF-DSGCLRVRCLSIQILMTLSLMQIQL-LVIVQLYIQLINGISLLVICTTLRLKMLQKKMTLKRVE
SLTFVGLYNKS-LLEVPWL-R-QNILGMLIFISSWDTSHGGQPLLLM-MRHHLKHF-LDVIILANHANK-MVMSCMQ
ITYFGGIQIQFSCLPILYLT-VNFPLN-GVLLLCL-KKVKSMI-FYLFLVKVDL-LEKTTELLFLVMELLTT
# 5′3′ Frame 2
CSPSYTVRHRH-Y-CRIQGF-HLQ--SSWFC-IPKN-LLSLPRKGRR-QFN-FLLCS-ETHFL-LPT-RNNL-FT-G
LSSCC-T-LL-V-NRR-HGTTYITSTSY-IHNGRPRLCFKAF--R-L-HIKRNTCHIQLL---LFQ-KGLV-FCRKP
RYITRIRQLR-TCTPSFVKNSTIL-CHAKCWYCWCTDIR-SRSQW-LV-FR-FHTNHAR-WSSCCRFLLFIVNAYIN
LDQGFNCRVTC-H-LNKALH-VGFVKI-LHGREVKTL-PLF-ILGSDIPPKLC-LFG-QMHSALCKL-CFILYSVPT
YKFWTTSEKNIC-WCSICSENWIPLORARCCT-SGCKLT-L-T-F-GITCVCC-PCYARCFW-SITR-THYVLFSSC
TY-QCCFSNCQTR-F-QRLL-LCCV-GFL-GRKFC-IKTLLLCSGW-CCYQRL-LLSL-STNNV-YQTTTICS-SC-
-VL-LLRWWLY-C-PSHRQQPRQISWFSI--MG-G-TLL-FNEL-GSRCTFRIYKT-CHPYYNSNES-VCH-CKE-S
SHRSWCLYL-YYDQ-TVSSKIIEINSRH-RSYCSNWNKQILWWLAQHVKNCL--CRKPSPYGLGLS-M--SHA-HA-
NYGLTCSCSQTYNVL-LVTPFL-IS--VCSSIE-NGHVWRFTIC-TRWNLIRRCHNCLC--CF-HLSSCHGQC-CTF
IY-W-QNCR-VCPQFTTOTL-VSL-K-RC-HRLCE-VLRIFA-TFLNDDTL-RCCCVFQ-HLCISRSSG-HKEL-VS
SLLSKQCFYV-SKMLD-D-PY-RTS-ILLSTYNAS-TG--LCVPSLPRSIKNPRGRLFCR-YRKNRWYTYD-TVRVF
SYRCLPTY-TS-SGVC-CLSFVLTIHKKAT--VNRTHVRHVFCYAY---HFKVLGT-VL-GYVHTAYSLTGCWGLCS
LQFTDFIKMWCLHT-TILML-MLLRPCHINIT-ISLVC-SVCLQCSRL-CHRCDSTLLRRYELLL-IT-TTH-FSIV
C-WTSFWFI-KYMCW-R-CY-L-CNCNM-LDKCW-LHES-HLY-KTOAFCSRNAQSY-GDI-TVLWYCYCT-SAV-Q
RITSFMGSW-T-TTT-PKLCLYWLSCN-KQ-STNRRVHL-KR-LW-CCCLPRYNNLQIKCW-LFCADITYSNAIKCT
YTSATRALC-NYWLIPNTQYLR-VF-QCCKLSKGWYAKVFYTPGTTWYW-ESFCYWPSSLLPFCSHSVYSLLSCRC-
CTM-EGIKIFAYR-M--NYTCTCSCRVF--IQSEFNIRTVCLLYCKCIA-DDSRYSCL--NFNGHKL-FECCQCQIT
C-ALCVHWRPCSITCTTHIAN-GHTRTRIFQFSV-TYENYRSRHVPRNLSALSC-NC-HCECFGL---A-ST-RQIS
SML-NVL-GCYHA-CFICN-QATNRRGKRIPYT-PCLEKSCLYFTL-FTECCSLKDFGTTNSNC-FITGLRI-LCHI
HSNH-NSSLL-CKQI-CCYYQSKSRHTLHNV--RPL-QVAIYKS-NST-ECGNFTS-KCNRTL-RL--GNHWVTSYT
GTYTPQC-H-IQN-RFMC-HTWHT-GHDL-KTHLYDGF-NELSS-WLP-HVYHPRRSYKTCTCMDWLRCRGVSCY-R
SCWYQFTFTARFFYRC-PSCCTYRLC-YT--YRFFQS-C-TTAWRSI-TPHTTYVORTSLECSAYKDCTNVK-HT-K
SL-QSRICLMGTWL-VDIYEVFCENRT-AHLLSM--TCHMLFHCFRHLCLLASFYWI-LRL-SVYD-CSTMGFYR-P
TKQP-SVLSSPW-CTCS-L-CNHD-VSSCPRVLC-AC-LDY-ISYNW--TED-CGL-KGSTHGC-SCIISRQIPSSS
RHW-P-SY-VCTSS-CRMEVL-CTAL--QSL-NRRIILFLCHTF-QIHRWCMPILELQCR-ISC-FHCL-I-H-SAI
-P-LAWL-WWQFVCK-TCIPHTSF--KCFC-FKTITIFLLL-QSM-VSWKTSSVRYRLCTTKVCYVYNTLQFRWCCL
-TSC--VOIVSRCL-HDDLSWL-LVGLQTI-YL-PLEHFYKTSEFRKCGF-CCK-GTL-WTTG-STSFYH--HCLHK
S-WC-CRIV-K-NNITC-CSI-ALG-AQH-TSTRGENTQ-FGCGHCC-YCDLGLOKRCSSTYIYYWCLFYD-HSQET
N-NDLCTTHCLF-W-S-WSSRLI-KCP-WCSYYRR-C-RFTTICRSQTS-S-WSHINWRSRKNTVOLL-ES-WCCPT
IT-NLLYSE-KFTRI-TQESNGN-FLRISYG-IH-TV-IRRLCLRTYRLWRF-S-SVRWFTSTDWTS-TF-GITF-I
RRFYSYGQYS-KLFHNRCANRFI-VCVFCY-FIT--FC-NNKIPRFICSF-GCQSDY-LYRNFIYALV-RWPCRNIL
PKITI-SSVATGCCYA-SLONAKNAIRKV-PSKLW--CNIT-RHNDECRKIYSTVSIFKHINISCTL-YESYTFWCW
F--RSCTRYSCFKTVVAYGYAACRFRS--LCL-CRFNFDW-LCNCTYS--MGSHY--YVRP-D-KCYKRK-L-RGFF
HLHLWVYTTKASSWRFRGYKDNRTFLEC-SL-AHGTLRMVDSLCY-CECVII-SIFNWM-LSWQTTRTNRWLCHACK
LHILEEYKSNSVVFLFFI-HE-ISP-IKGYCCYVFKRRSNQ-YDFISS--R-TYN-RKQQSCYF--CSC-QL
# 5′3′ Frame 3 (longest ORF)
AARLTPCGTGTSTDVVYRAFDIYNDKVAGFAKFLKTNCCRFQEKDEDDNLIDSYFVVKRHTFSNYQHEETIYNLLKD
CPAVAKHDFFKFRIDGDMVPHISRORLTKYTMADLVYALRHFDEGNCDTLKEILVTYNCCDDDYENKKDWYDFVENP
DILRVYANLGERVRQALLKTVQFCDAMRNAGIVGVLTLDNQDLNGNWYDFGDFIQTTPGSGVPVVDSYYSLLMPILT
LTRALTAESHVDTDLTKPYIKWDLLKYDFTEERLKLFDRYFKYWDQTYHPNCVNCLDDRCILHCANFNVLFSTVFPP
TSFGPLVRKIFVDGVPFVVSTGYHFRELGVVHNQDVNLHSSRLSFKELLVYAADPAMHAASGNLLLDKRTTCFSVAA
LTNNVAFQTVKPGNFNKDFYDFAVSKGFFKEGSSVELKHFFFAQDGNAAISDYDYYRYNLPTMCDIRQLLFVVEVVD
KYFDCYDGGCINANQVIVNNLDKSAGFPFNKWGKARLYYDSMSYEDQDALFAYTKRNVIPTITQMNLKYAISAKNRA
RTVAGVSICSTMTNRQFHQKLLKSIAATRGATVVIGTSKFYGGWHNMLKTVYSDVENPHLMGWDYPKCDRAMPNMLR
IMASLVLARKHTTCCSLSHRFYRLANECAQVLSEMVMCGGSLYVKPGGTSSGDATTAYANSVENICQAVTANVNALL
STDGNKIADKYVRNLQHRLYECLYRNRDVDTDFVNEFYAYLRKHFSMMILSDDAVVCENSTYASQGLVASIKNFKSV
LYYQNNVFMSEAKCWTETDLTKGPHEFCSQHTMLVKQGDDYVYLPYPDPSRILGAGCFVDDIVKTDGTLMIERFVSL
AIDAYPLTKHPNQEYADVFHLYLQYIRKLHDELTGHMLDMYSVMLINDNTSRYWEPEFYEAMYTPHTVLQAVGACVL
CNSQTSLRCGACIRRPFLCCKCCYDHVISTSHKLVLSVNPYVCNAPGCDVTDVTOLYLGGMSYYCKSHKPPISFPLC
ANGQVFGLYKNTCVGSDNVTDFNAIATCDWTNAGDYILANTCTERLKLFAAETLKATEETFKLSYGIATVREVLSDR
ELHLSWEVGKPRPPLNRNYVFTGYRVTKNSKVQIGEYTFEKGDYGDAVVYRGTTTYKLNVGDYFVLTSHTVMPLSAP
TLVPQEHYVRITGLYPTLNISDEFSSNVANYQKVGMQKYSTLQGPPGTGKSHFAIGLALYYPSARIVYTACSHAAVD
ALCEKALKYLPIDKCSRIIPARARVECFDKFKVNSTLEQYVFCTVNALPETTADIVVFDEISMATNYDLSVVNARLR
AKHYVYIGDPAQLPAPRTLLTKGTLEPEYFNSVCRLMKTIGPDMFLGTCRRCPAEIVDTVSALVYDNKLKAHKDKSA
QCFKMFYKGVITHDVSSAINRPQIGVVREFLTRNPAWRKAVFISPYNSQNAVASKILGLPTQTVDSSQGSEYDYVIF
TQTTETAHSCNVNRENVAITRAKVGILCIMSDRDLYDKLOFTSLEIPRRNVATLQAENVTGLFKDCSKVITGLHPTQ
APTHLSVDTKFKTEGLCVDIPGIPKDMTYRRLISMMGFKMNYQVNGYPNMFITREEAIRHVRAWIGEDVEGCHATRE
AVGTNLPLQLGFSTGVNLVAVPTGYVDTPNNTDFSRVSAKPPPGDOFKHLIPLMYKGLPWNVVRIKIVOMLSDTLKN
LSDRVVFVLWAHGFELTSMKYFVKIGPERTCCLCDRRATCESTASDTYACWHHSIGFDYVYNPFMIDVQQWGFTGNL
QSNHDLYCQVHGNAHVASCDAIMTRCLAVHECFVKRVDWTIEYPIIGDELKINAACRKVQHMVVKAALLADKFPVLH
DIGNPKAIKCVPQADVEWKFYDAQPCSDKAYKIEELFYSYATHSDKFTDGVCLFWNCNVDRYPANSIVCREDTRVLS
NLNLPGCDGGSLYVNKHAFHTPAFDKSAFVNLKQLPFFYYSDSPCESHGKQVVSDIDYVPLKSATCITRONLGGAVC
RHHANEYRLYLDAYNMMISAGFSLWVYKQFDTYNLWNTFTRLQSLENVAFNVVNKGHFDGQQGEVPVSIINNTVYTK
VDGVDVELFENKTTLPVNVAFELWAKRNIKPVPEVKILNNLGVDIAANTVIWDYKRDAPAHISTIGVCSMTDIAKKP
TETICAPLTVFFDGRVDGQVDLFRNARNGVLITEGSVKGLQPSVGPKQASLNGVTLIGEAVKTQFNYYKKVDGVVQQ
LPETYFTQSRNLQEFKPRSQMEIDFLELAMDEFIERYKLEGYAFEHIVYGDFSHSQLGGLHLLIGLAKRFKESPFEL
EDFIPMDSTVKNYFITDAQTGSSKCVCSVIDLLLDDFVEIIKSQDLSVVSKVVKVTIDYTEISFMLWCKDGHVETFY
PKLOSSQAWQPGVAMPNLYKMQRMLLEKCDLQNYGDSATLPKGIMMNVAKYTQLCQYLNTLTLAVPYNMRVIHFGAG
SDKGVAPGTAVLRQWLPTGTLLVDSDLNDFVSDADSTLIGDCATVHTANKWDLIISDMYDPKTKNVTKENDSKEGFF
TYICGFIQQKLALGGSVAIKITEHSWNADLYKLMGHFAWWTAFVTNVNASSSEAFLIGCNYLGKPREQIDGYVMHAN
YIFWRNTNPIQLSSYSLFDMSKFPLKLRGTAVMSLKEGQINDMILSLLSKGRLIIRENNRVVISSDVLVNN

The nucleotide sequence of the spike protein(S) (Severe acute respiratory syndrome coronavirus 2; GenBank: QHD43416.1; SEQ ID NO: 8) is:

atggatttgtttatgagaatcttcacaattggaactgtaactttgaagca
aggtgaaatcaaggatgctactccttcagattttgttcgcgctactgcaa
cgataccgatacaagcctcactccctttcggatggcttattgttggcgtt
gcacttcttgctgtttttcagagcgcttccaaaatcataaccctcaaaaa
gagatggcaactagcactctccaagggtgttcactttgtttgcaacttgc
tgttgttgtttgtaacagtttactcacaccttttgctcgttgctgctggc
cttgaagccccttttctctatctttatgctttagtctacttcttgcagag
tataaactttgtaagaataataatgaggctttggctttgctggaaatgcc
gttccaaaaacccattactttatgatgccaactattttctttgctggcat
actaattgttacgactattgtataccttacaatagtgtaacttcttcaat
tgtcattacttcaggtgatggcacaacaagtcctatttctgaacatgact
accagattggtggttatactgaaaaatgggaatctggagtaaaagactgt
gttgtattacacagttacttcacttcagactattaccagctgtactcaac
tcaattgagtacagacactggtgttgaacatgttaccttcttcatctaca
ataaaattgttgatgagcctgaagaacatgtccaaattcacacaatcgac
ggttcatccggagttgttaatccagtaatggaaccaatttatgatgaacc
gacgacgactactagcgtgcctttgtaa

The nucleotide sequence of the membrane (M) protein (Severe acute respiratory syndrome coronavirus 2; GeneBank: QHD43419.1; SEQ ID NO: 9) is:

atgtttcatctcgttgactttcaggttactatagcagagatattactaat
tattatgaggacttttaaagtttccatttggaatcttgattacatcataa
acctcataattaaaaatttatctaagtcactaactgagaataaatattct
caattagatgaagagcaaccaatggagattgattaa

The nucleotide sequence of the envelope (E) protein (Severe acute respiratory syndrome coronavirus 2; GeneBank Accession No. QHD43418.1; SEQ ID NO: 10) is:

atggcagattccaacggtactattaccgttgaagagcttaaaaagctcct
tgaacaatggaacctagtaataggtttcctattccttacatggatttgtc
ttctacaatttgcctatgccaacaggaataggtttttgtatataattaag
ttaattttcctctggctgttatggccagtaactttagcttgttttgtgct
tgctgctgtttacagaataaattggatcaccggtggaattgctatcgcaa
tggcttgtcttgtaggcttgatgtggctcagctacttcattgcttctttc
agactgtttgcgcgtacgcgttccatgtggtcattcaatccagaaactaa
cattcttctcaacgtgccactccatggcactattctgaccagaccgcttc
tagaaagtgaactcgtaatcggagctgtgatccttcgtggacatcttcgt
attgctggacaccatctaggacgctgtgacatcaaggacctgcctaaaga
aatcactgttgctacatcacgaacgctttcttattacaaattgggagctt
cgcagcgtgtagcaggtgactcaggttttgctgcatacagtcgctacagg
attggcaactataaattaaacacagaccattccagtagcagtgacaatat
tgctttgcttgtacagtaa

The nucleotide sequence of the nucleocapsid (N) protein (Severe acute respiratory syndrome coronavirus 2; GeneBank: QHD43423.2; SEQ ID NO: 11) is:

atgtctgataatggaccccaaaatcagcgaaatgcaccccgcattacgtt
tggtggaccctcagattcaactggcagtaaccagaatggagaacgcagtg
gggcgcgatcaaaacaacgtcggccccaaggtttacccaataatactgcg
tcttggttcaccgctctcactcaacatggcaaggaagaccttaaattccc
tcgaggacaaggcgttccaattaacaccaatagcagtccagatgaccaaa
ttggctactaccgaagagctaccagacgaattcgtggtggtgacggtaaa
atgaaagatctcagtccaagatggtatttctactacctaggaactgggcc
agaagctggacttccctatggtgctaacaaagacggcatcatatgggttg
caactgagggagccttgaatacaccaaaagatcacattggcacccgcaat
cctgctaacaatgctgcaatcgtgctacaacttcctcaaggaacaacatt
gccaaaaggcttctacgcagaagggagcagaggcggcagtcaagcctctt
ctcgttcctcatcacgtagtcgcaacagttcaagaaattcaactccaggc
agcagtaggggaacttctcctgctagaatggctggcaatggcggtgatgc
tgctcttgctttgctgctgcttgacagattgaaccagcttgagagcaaaa
tgtctggtaaaggccaacaacaacaaggccaaactgtcactaagaaatct
gctgctgaggcttctaagaagcctcggcaaaaacgtactgccactaaagc
atacaatgtaacacaagctttcggcagacgtggtccagaacaaacccaag
gaaattttggggaccaggaactaatcagacaaggaactgattacaaacat
tggccgcaaattgcacaatttgcccccagcgcttcagcgttcttcggaat
gtcgcgcattggcatggaagtcacaccttcgggaacgtggttgacctaca
caggtgccatcaaattggatgacaaagatccaaatttcaaagatcaagtc
attttgctgaataagcatattgacgcatacaaaacattcccaccaacaga
gcctaaaaaggacaaaaagaagaaggctgatgaaactcaagccttaccgc
agagacagaagaaacagcaaactgtgactcttcttcctgctgcagatttg
gatgatttctccaaacaattgcaacaatccatgagcagtgctgactcaac
tcaggcctaa

The nucleotide sequence for the protein coding sequence (CDS) of the helicase (H) (Severe acute respiratory syndrome coronavirus 2; NCBI Reference Sequence: YP_009725308.1; SEQ ID NO: 12)

gctgttggggcttgtgttctttgcaattcacagacttcattaagatgtgg
tgcttgcatacgtagaccattcttatgttgtaaatgctgttacgaccatg
tcatatcaacatcacataaattagtcttgtctgttaatccgtatgtttgc
aatgctccaggttgtgatgtcacagatgtgactcaactttacttaggagg
tatgagctattattgtaaatcacataaaccacccattagttttccattgt
gtgctaatggacaagtttttggtttatataaaaatacatgtgttggtagc
gataatgttactgactttaatgcaattgcaacatgtgactggacaaatgc
tggtgattacattttagctaacacctgtactgaaagactcaagctttttg
cagcagaaacgctcaaagctactgaggagacatttaaactgtcttatggt
attgctactgtacgtgaagtgctgtctgacagagaattacatctttcatg
ggaagttggtaaacctagaccaccacttaaccgaaattatgtctttactg
gttatcgtgtaactaaaaacagtaaagtacaaataggagagtacaccttt
gaaaaaggtgactatggtgatgctgttgtttaccgaggtacaacaactta
caaattaaatgttggtgattattttgtgctgacatcacatacagtaatgc
cattaagtgcacctacactagtgccacaagagcactatgttagaattact
ggcttatacccaacactcaatatctcagatgagttttctagcaatgttgc
aaattatcaaaaggttggtatgcaaaagtattctacactccagggaccac
ctggtactggtaagagtcattttgctattggcctagctctctactaccct
tctgctcgcatagtgtatacagcttgctctcatgccgctgttgatgcact
atgtgagaaggcattaaaatatttgcctatagataaatgtagtagaatta
tacctgcacgtgctcgtgtagagtgttttgataaattcaaagtgaattca
acattagaacagtatgtcttttgtactgtaaatgcattgcctgagacgac
agcagatatagttgtctttgatgaaatttcaatggccacaaattatgatt
tgagtgttgtcaatgccagattacgtgctaagcactatgtgtacattggc
gaccctgctcaattacctgcaccacgcacattgctaactaagggcacact
agaaccagaatatttcaattcagtgtgtagacttatgaaaactataggtc
cagacatgttcctcggaacttgtcggcgttgtcctgctgaaattgttgac
actgtgagtgctttggtttatgataataagcttaaagcacataaagacaa
atcagctcaatgctttaaaatgttttataagggtgttatcacgcatgatg
tttcatctgcaattaacaggccacaaataggcgtggtaagagaattcctt
acacgtaaccctgcttggagaaaagctgtctttatttcaccttataattc
acagaatgctgtagcctcaaagattttgggactaccaactcaaactgttg
attcatcacagggctcagaatatgactatgtcatattcactcaaaccact
gaaacagctcactcttgtaatgtaaacagatttaatgttgctattaccag
agcaaaagtaggcatactttgcataatgtctgatagagacctttatgaca
agttgcaatttacaagtcttgaaattccacgtaggaatgtggcaacttta
caa

The nucleotide sequence for the CDS of RNA-Dependent RNA polymerase (RdRp) (Severe acute respiratory syndrome coronavirus 2; NCBI Reference Sequence: YP 009725307.1; SEQ ID NO: 13) is:

tcagctgatgcacaatcgtttttaaacgggtttgcggtgtaagtgcagcc
cgtcttacaccgtgcggcacaggcactagtactgatgtcgtatacagggc
ttttgacatctacaatgataaagtagctggttttgctaaattcctaaaaa
ctaattgttgtcgcttccaagaaaaggacgaagatgacaatttaattgat
tcttactttgtagttaagagacacactttctctaactaccaacatgaaga
aacaatttataatttacttaaggattgtccagctgttgctaaacatgact
tctttaagtttagaatagacggtgacatggtaccacatatatcacgtcaa
cgtcttactaaatacacaatggcagacctcgtctatgctttaaggcattt
tgatgaaggtaattgtgacacattaaaagaaatacttgtcacatacaatt
gttgtgatgatgattatttcaataaaaaggactggtatgattttgtagaa
aacccagatatattacgcgtatacgccaacttaggtgaacgtgtacgcca
agctttgttaaaaacagtacaattctgtgatgccatgcgaaatgctggta
ttgttggtgtactgacattagataatcaagatctcaatggtaactggtat
gatttcggtgatttcatacaaaccacgccaggtagtggagttcctgttgt
agattcttattattcattgttaatgcctatattaaccttgaccagggctt
taactgcagagtcacatgttgacactgacttaacaaagccttacattaag
tgggatttgttaaaatatgacttcacggaagagaggttaaaactctttga
ccgttattttaaatattgggatcagacataccacccaaattgtgttaact
gtttggatgacagatgcattctgcattgtgcaaactttaatgttttattc
tctacagtgttcccacctacaagttttggaccactagtgagaaaaatatt
tgttgatggtgttccatttgtagtttcaactggataccacttcagagagc
taggtgttgtacataatcaggatgtaaacttacatagctctagacttagt
tttaaggaattacttgtgtatgctgctgaccctgctatgcacgctgcttc
tggtaatctattactagataaacgcactacgtgcttttcagtagctgcac
ttactaacaatgttgcttttcaaactgtcaaacccggtaattttaacaaa
gacttctatgactttgctgtgtctaagggtttctttaaggaaggaagttc
tgttgaattaaaacacttcttctttgctcaggatggtaatgctgctatca
gcgattatgactactatcgttataatctaccaacaatgtgtgatatcaga
caactactatttgtagttgaagttgttgataagtactttgattgttacga
tggtggctgtattaatgctaaccaagtcatcgtcaacaacctagacaaat
cagctggttttccatttaataaatggggtaaggctagactttattatgat
tcaatgagttatgaggatcaagatgcacttttcgcatatacaaaacgtaa
tgtcatccctactataactcaaatgaatcttaagtatgccattagtgcaa
agaatagagctcgcaccgtagctggtgtctctatctgtagtactatgacc
aatagacagtttcatcaaaaattattgaaatcaatagccgccactagagg
agctactgtagtaattggaacaagcaaattctatggtggttggcacaaca
tgttaaaaactgtttatagtgatgtagaaaaccctcaccttatgggttgg
gattatcctaaatgtgatagagccatgcctaacatgcttagaattatggc
ctcacttgttcttgctcgcaaacatacaacgtgttgtagcttgtcacacc
gtttctatagattagctaatgagtgtgctcaagtattgagtgaaatggtc
atgtgtggcggttcactatatgttaaaccaggtggaacctcatcaggaga
tgccacaactgcttatgctaatagtgtttttaacatttgtcaagctgtca
cggccaatgttaatgcacttttatctactgatggtaacaaaattgccgat
aagtatgtccgcaatttacaacacagactttatgagtgtctctatagaaa
tagagatgttgacacagactttgtgaatgagttttacgcatatttgcgta
aacatttctcaatgatgatactctctgacgatgctgttgtgtgtttcaat
agcacttatgcatctcaaggtctagtggctagcataaagaactttaagtc
agttctttattatcaaaacaatgtttttatgtctgaagcaaaatgttgga
ctgagactgaccttactaaaggacctcatgaattttgctctcaacataca
atgctagttaaacagggtgatgattatgtgtaccttccttacccagatcc
atcaagaatcctaggggccggctgttttgtagatgatatcgtaaaaacag
atggtacacttatgattgaacggttcgtgtctttagctatagatgcttac
ccacttactaaacatcctaatcaggagtatgctgatgtctttcatttgta
cttacaatacataagaaagctacatgatgagttaacaggacacatgttag
acatgtattctgttatgcttactaatgataacacttcaaggtattgggaa
cctgagttttatgaggctatgtacacaccgcatacagtcttacag

The nucleic sequence of ORF1b) (Severe acute respiratory syndrome coronavirus 2; NCBI Sequence: BCN86436.1; SEQ ID NO: 14) is:

gtgcagcccgtcttacaccgtgcggcacaggcactagtactgatgtcgtatacagggcttttgacatctacaatgat
aaagtagctggttttgctaaattcctaaaaactaattgttgtcgcttccaagaaaaggacgaagatgacaatttaat
tgattcttactttgtagttaagagacacactttctctaactaccaacatgaagaaacaatttataatttacttaagg
attgtccagctgttgctaaacatgacttctttaagtttagaatagacggtgacatggtaccacatatatcacgtcaa
cgtcttactaaatacacaatggcagacctcgtctatgctttaaggcattttgatgaaggtaattgtgacacattaaa
agaaatacttgtcacatacaattgttgtgatgatgattatttcaataaaaaggactggtatgattttgtagaaaacc
cagatatattacgcgtatacgccaacttaggtgaacgtgtacgccaagctttgttaaaaacagtacaattctgtgat
gccatgcgaaatgctggtattgttggtgtactgacattagataatcaagatctcaatggtaactggtatgatttcgg
tgatttcatacaaaccacgccaggtagtggagttcctgttgtagattcttattattcattgttaatgcctatattaa
ccttgaccagggctttaactgcagagtcacatgttgacactgacttaacaaagccttacattaagtgggatttgtta
aaatatgacttcacggaagagaggttaaaactctttgaccgttattttaaatattgggatcagacataccacccaaa
ttgtgttaactgtttggatgacagatgcattctgcattgtgcaaactttaatgttttattctctacagtgttcccac
ctacaagttttggaccactagtgagaaaaatatttgttgatggtgttccatttgtagtttcaactggataccacttc
agagagctaggtgttgtacataatcaggatgtaaacttacatagctctagacttagttttaaggaattacttgtgta
tgctgctgaccctgctatgcacgctgcttctggtaatctattactagataaacgcactacgtgcttttcagtagctg
cacttactaacaatgttgcttttcaaactgtcaaacccggtaattttaacaaagacttctatgactttgctgtgtct
aagggtttctttaaggaaggaagttctgttgaattaaaacacttcttctttgctcaggatggtaatgctgctatcag
cgattatgactactatcgttataatctaccaacaatgtgtgatatcagacaactactatttgtagttgaagttgttg
ataagtactttgattgttacgatggtggctgtattaatgctaaccaagtcatcgtcaacaacctagacaaatcagct
ggttttccatttaataaatggggtaaggctagactttattatgattcaatgagttatgaggatcaagatgcactttt
cgcatatacaaaacgtaatgtcatccctactataactcaaatgaatcttaagtatgccattagtgcaaagaatagag
ctcgcaccgtagctggtgtctctatctgtagtactatgaccaatagacagtttcatcaaaaattattgaaatcaata
gccgccactagaggagctactgtagtaattggaacaagcaaattctatggtggttggcacaacatgttaaaaactgt
ttatagtgatgtagaaaaccctcaccttatgggttgggattatcctaaatgtgatagagccatgcctaacatgctta
gaattatggcctcacttgttcttgctcgcaaacatacaacgtgttgtagcttgtcacaccgtttctatagattagct
aatgagtgtgctcaagtattgagtgaaatggtcatgtgtggcggttcactatatgttaaaccaggtggaacctcatc
aggagatgccacaactgcttatgctaatagtgtttttaacatttgtcaagctgtcacggccaatgttaatgcacttt
tatctactgatggtaacaaaattgccgataagtatgtccgcaatttacaacacagactttatgagtgtctctataga
aatagagatgttgacacagactttgtgaatgagttttacgcatatttgcgtaaacatttctcaatgatgatactctc
tgacgatgctgttgtgtgtttcaatagcacttatgcatctcaaggtctagtggctagcataaagaactttaagtcag
ttctttattatcaaaacaatgtttttatgtctgaagcaaaatgttggactgagactgaccttactaaaggacctcat
gaattttgctctcaacatacaatgctagttaaacagggtgatgattatgtgtaccttccttacccagatccatcaag
aatcctaggggccggctgttttgtagatgatatcgtaaaaacagatggtacacttatgattgaacggttcgtgtctt
tagctatagatgcttacccacttactaaacatcctaatcaggagtatgctgatgtctttcatttgtacttacaatac
ataagaaagctacatgatgagttaacaggacacatgttagacatgtattctgttatgcttactaatgataacacttc
aaggtattgggaacctgagttttatgaggctatgtacacaccgcatacagtcttacaggctgttggggcttgtgttc
tttgcaattcacagacttcattaagatgtggtgcttgcatacgtagaccattcttatgttgtaaatgctgttacgac
catgtcatatcaacatcacataaattagtcttgtctgttaatccgtatgtttgcaatgctccaggttgtgatgtcac
agatgtgactcaactttacttaggaggtatgagctattattgtaaatcacataaaccacccattagttttccattgt
gtgctaatggacaagtttttggtttatataaaaatacatgtgttggtagcgataatgttactgactttaatgcaatt
gcaacatgtgactggacaaatgctggtgattacattttagctaacacctgtactgaaagactcaagctttttgcagc
agaaacgctcaaagctactgaggagacatttaaactgtcttatggtattgctactgtacgtgaagtgctgtctgaca
gagaattacatctttcatgggaagttggtaaacctagaccaccacttaaccgaaattatgtctttactggttatcgt
gtaactaaaaacagtaaagtacaaataggagagtacacctttgaaaaaggtgactatggtgatgctgttgtttaccg
aggtacaacaacttacaaattaaatgttggtgattattttgtgctgacatcacatacagtaatgccattaagtgcac
ctacactagtgccacaagagcactatgttagaattactggcttatacccaacactcaatatctcagatgagttttct
agcaatgttgcaaattatcaaaaggttggtatgcaaaagtattctacactccagggaccacctggtactggtaagag
tcattttgctattggcctagctctctactacccttctgctcgcatagtgtatacagcttgctctcatgccgctgttg
atgcactatgtgagaaggcattaaaatatttgcctatagataaatgtagtagaattatacctgcacgtgctcgtgta
gagtgttttgataaattcaaagtgaattcaacattagaacagtatgtcttttgtactgtaaatgcattgcctgagac
gacagcagatatagttgtctttgatgaaatttcaatggccacaaattatgatttgagtgttgtcaatgccagattac
gtgctaagcactatgtgtacattggcgaccctgctcaattacctgcaccacgcacattgctaactaagggcacacta
gaaccagaatatttcaattcagtgtgtagacttatgaaaactataggtccagacatgttcctcggaacttgtcggcg
ttgtcctgctgaaattgttgacactgtgagtgctttggtttatgataataagcttaaagcacataaagacaaatcag
ctcaatgctttaaaatgttttataagggtgttatcacgcatgatgtttcatctgcaattaacaggccacaaataggc
gtggtaagagaattccttacacgtaaccctgcttggagaaaagctgtctttatttcaccttataattcacagaatgc
tgtagcctcaaagattttgggactaccaactcaaactgttgattcatcacagggctcagaatatgactatgtcatat
tcactcaaaccactgaaacagctcactcttgtaatgtaaacagatttaatgttgctattaccagagcaaaagtaggc
atactttgcataatgtctgatagagacctttatgacaagttgcaatttacaagtcttgaaattccacgtaggaatgt
ggcaactttacaagctgaaaatgtaacaggactctttaaagattgtagtaaggtaatcactgggttacatcctacac
aggcacctacacacctcagtgttgacactaaattcaaaactgaaggtttatgtgttgacatacctggcatacctaag
gacatgacctatagaagactcatctctatgatgggttttaaaatgaattatcaagttaatggttaccctaacatgtt
tatcacccgcgaagaagctataagacatgtacgtgcatggattggcttcgatgtcgaggggtgtcatgctactagag
aagctgttggtaccaatttacctttacagctaggtttttctacaggtgttaacctagttgctgtacctacaggttat
gttgatacacctaataatacagatttttccagagttagtgctaaaccaccgcctggagatcaatttaaacacctcat
accacttatgtacaaaggacttccttggaatgtagtgcgtataaagattgtacaaatgttaagtgacacacttaaaa
atctctctgacagagtcgtatttgtcttatgggcacatggctttgagttgacatctatgaagtattttgtgaaaata
ggacctgagcgcacctgttgtctatgtgatagacgtgccacatgcttttccactgcttcagacacttatgcctgttg
gcatcattctattggatttgattacgtctataatccgtttatgattgatgttcaacaatggggttttacaggtaacc
tacaaagcaaccatgatctgtattgtcaagtccatggtaatgcacatgtagctagttgtgatgcaatcatgactagg
tgtctagctgtccacgagtgctttgttaagcgtgttgactggactattgaatatcctataattggtgatgaactgaa
gattaatgcggcttgtagaaaggttcaacacatggttgttaaagctgcattattagcagacaaattcccagttcttc
acgacattggtaaccctaaagctattaagtgtgtacctcaagctgatgtagaatggaagttctatgatgcacagcct
tgtagtgacaaagcttataaaatagaagaattattctattcttatgccacacattctgacaaattcacagatggtgt
atgcctattttggaattgcaatgtcgatagatatcctgctaattccattgtttgtagatttgacactagagtgctat
ctaaccttaacttgcctggttgtgatggtggcagtttgtatgtaaataaacatgcattccacacaccagcttttgat
aaaagtgcttttgttaatttaaaacaattaccatttttctattactctgacagtccatgtgagtctcatggaaaaca
agtagtgtcagatatagattatgtaccactaaagtctgctacgtgtataacacgttgcaatttaggtggtgctgtct
gtagacatcatgctaatgagtacagattgtatctcgatgcttataacatgatgatctcagctggctttagcttgtgg
gtttacaaacaatttgatacttataacctctggaacacttttacaagacttcagagtttagaaaatgtggcttttaa
tgttgtaaataagggacactttgatggacaacagggtgaagtaccagtttctatcattaataacactgtttacacaa
aagttgatggtgttgatgtagaattgtttgaaaataaaacaacattacctgttaatgtagcatttgagctttgggct
aagcgcaacattaaaccagtaccagaggtgaaaatactcaataatttgggtgtggacattgctgctaatactgtgat
ctgggactacaaaagagatgctccagcacatatatctactattggtgtttgttctatgactgacatagccaagaaac
caactgaaacgatttgtgcaccactcactgtcttttttgatggtagagttgatggtcaagtagacttatttagaaat
gcccgtaatggtgttcttattacagaaggtagtgttaaaggtttacaaccatctgtaggtcccaaacaagctagtct
taatggagtcacattaattggagaagccgtaaaaacacagttcaattattataagaaagttgatggtgttgtccaac
aattacctgaaacttactttactcagagtagaaatttacaagaatttaaacccaggagtcaaatggaaattgatttc
ttagaattagctatggatgaattcattgaacggtataaattagaaggctatgccttcgaacatatcgtttatggaga
ttttagtcatagtcagttaggtggtttacatctactgattggactagctaaacgttttaaggaatcaccttttgaat
tagaagattttattcctatggacagtacagttaaaaactatttcataacagatgcgcaaacaggttcatctaagtgt
gtgtgttctgttattgatttattacttgatgattttgttgaaataataaaatcccaagatttatctgtagtttctaa
ggttgtcaaagtgactattgactatacagaaatttcatttatgctttggtgtaaagatggccatgtagaaacatttt
acccaaaattacaatctagtcaagcgtggcaaccgggtgttgctatgcctaatctttacaaaatgcaaagaatgcta
ttagaaaagtgtgaccttcaaaattatggtgatagtgcaacattacctaaaggcataatgatgaatgtcgcaaaata
tactcaactgtgtcaatatttaaacacattaacattagctgtaccctataatatgagagttatacattttggtgctg
gttctgataaaggagttgcaccaggtacagctgttttaagacagtggttgcctacgggtacgctgcttgtcgattca
gatcttaatgactttgtctctgatgcagattcaactttgattggtgattgtgcaactgtacatacagctaataaatg
ggatctcattattagtgatatgtacgaccctaagactaaaaatgttacaaaagaaaatgactctaaagagggttttt
tcacttacatttgtgggtttatacaacaaaagctagctcttggaggttccgtggctataaagataacagaacattct
tggaatgctgatctttataagctcatgggacacttcgcatggtggacagcctttgttactaatgtgaatgcgtcatc
atctgaagcatttttaattggatgtaattatcttggcaaaccacgcgaacaaatagatggttatgtcatgcatgcaa
attacatattttggaggaatacaaatccaattcagttgtcttcctattctttatttgacatgagtaaatttcccctt
aaattaaggggtactgctgttatgtctttaaaagaaggtcaaatcaatgatatgattttatctcttcttagtaaagg
tagacttataattagagaaaacaacagagttgttatttctagtgatgttcttgttaacaactaa

The amino sequence of ORF1a (SEQ ID NO: 15) is:

# 5′3′ Frame 1 (longest ORF)
MESLVPGFNEKTHVQLSLPVLQVRDVLVRGFGDSVEEVLSEARQHLKDGTCGLVEVEKGVLPOLEQPYVFIKRSDAR
TAPHGHVMVELVAELEGIQYGRSGETLGVLVPHVGEIPVAYRKVLLRKNGNKGAGGHSYGADLKSFDLGDELGTDPY
EDFQENWNTKHSSGVTRELMRELNGGAYTRYVDNNFCGPDGYPLECIKDLLARAGKASCTLSEQLDFIDTKRGVYCC
REHEHEIAWYTERSEKSYELQTPFEIKLAKKFDTENGECPNFVFPLNSIIKTIQPRVEKKKLDGFMGRIRSVYPVAS
PNECNQMCLSTLMKCDHCGETSWQTGDFVKATCEFCGTENLTKEGATTCGYLPQNAVVKIYCPACHNSEVGPEHSLA
EYHNESGLKTILRKGGRTIAFGGCVFSYVGCHNKCAYWVPRASANIGCNHTGVVGEGSEGLNDNLLEILOKEKVNIN
IVGDFKLNEEIAIILASFSASTSAFVETVKGLDYKAFKQIVESCGNFKVTKGKAKKGAWNIGEQKSILSPLYAFASE
AARVVRSIFSRTLETAQNSVRVLQKAAITILDGISQYSLRLIDAMMFTSDLATNNLVVMAYITGGVVOLTSQWLTNI
FGTVYEKLKPVLDWLEEKFKEGVEFLRDGWEIVKFISTCACEIVGGQIVTCAKEIKESVQTFFKLVNKFLALCADSI
IIGGAKLKALNLGETFVTHSKGLYRKCVKSREETGLLMPLKAPKEIIFLEGETLPTEVLTEEVVLKTGDLQPLEQPT
SEAVEAPLVGTPVCINGLMLLEIKDTEKYCALAPNMMVTNNTFTLKGGAPTKVTFGDDTVIEVQGYKSVNITFELDE
RIDKVLNEKCSAYTVELGTEVNEFACVVADAVIKTLQPVSELLTPLGIDLDEWSMATYYLFDESGEFKLASHMYCSF
YPPDEDEEEGDCEEEEFEPSTQYEYGTEDDYQGKPLEFGATSAALQPEEEQEEDWLDDDSQQTVGQQDGSEDNQTTT
IQTIVEVQPQLEMELTPVVQTIEVNSFSGYLKLTDNVYIKNADIVEEAKKVKPTVVVNAANVYLKHGGGVAGALNKA
TNNAMQVESDDYIATNGPLKVGGSCVLSGHNLAKHCLHVVGPNVNKGEDIQLLKSAYENFNQHEVLLAPLLSAGIFG
ADPIHSLRVCVDTVRTNVYLAVFDKNLYDKLVSSFLEMKSEKQVEQKIAEIPKEEVKPFITESKPSVEQRKODDKKI
KACVEEVTTTLEETKELTENLLLYIDINGNLHPDSATLVSDIDITFLKKDAPYIVGDVVQEGVLTAVVIPTKKAGGT
TEMLAKALRKVPTDNYITTYPGQGLNGYTVEEAKTVLKKCKSAFYILPSIISNEKQEILGTVSWNLREMLAHAEETR
KLMPVCVETKAIVSTIQRKYKGIKIQEGVVDYGARFYFYTSKTTVASLINTLNDLNETLVTMPLGYVTHGLNLEEAA
RYMRSLKVPATVSVSSPDAVTAYNGYLTSSSKTPEEHFIETISLAGSYKDWSYSGQSTQLGIEFLKRGDKSVYYTSN
PTTFHLDGEVITFDNLKTLLSLREVRTIKVFTTVDNINLHTQVVDMSMTYGQQFGPTYLDGADVTKIKPHNSHEGKT
FYVLPNDDTLRVEAFEYYHTTDPSFLGRYMSALNHTKKWKYPQVNGLTSIKWADNNCYLATALLTLQQIELKENPPA
LQDAYYRARAGEAANFCALILAYCNKTVGELGDVRETMSYLFQHANLDSCKRVLNVVCKTCGQQQTTLKGVEAVMYM
GTLSYEQFKKGVQIPCTCGKQATKYLVQQESPFVMMSAPPAQYELKHGTFTCASEYTGNYQCGHYKHITSKETLYCI
DGALLTKSSEYKGPITDVFYKENSYTTTIKPVTYKLDGVVCTEIDPKLDNYYKKDNSYFTEQPIDLVPNQPYPNASF
DNFKFVCDNIKFADDLNQLTGYKKPASRELKVTFFPDLNGDVVAIDYKHYTPSFKKGAKLLHKPIVWHVNNATNKAT
YKPNTWCIRCLWSTKPVETSNSFDVLKSEDAQGMDNLACEDLKPVSEEVVENPTIQKDVLECNVKTTEVVGDIILKP
ANNSLKITEEVGHTDLMAAYVDNSSLTIKKPNELSRVLGLKTLATHGLAAVNSVPWDTIANYAKPFLNKVVSTTTNI
VTRCLNRVCTNYMPYFFTLLLQLCTFTRSTNSRIKASMPTTIAKNTVKSVGKFCLEASFNYLKSPNFSKLINIIIWF
LLLSVCLGSLIYSTAALGVLMSNLGMPSYCTGYREGYLNSTNVTIATYCTGSIPCSVCLSGLDSLDTYPSLETIQIT
ISSFKWDLTAFGLVAEWFLAYILFTRFFYVLGLAAIMQLFFSYFAVHFISNSWLMWLIINLVQMAPISAMVRMYIFF
ASFYYVWKSYVHVVDGCNSSTCMMCYKRNRATRVECTTIVNGVRRSFYVYANGGKGFCKLHNWNCVNCDTFCAGSTF
ISDEVARDLSLQFKRPINPTDQSSYIVDSVTVKNGSIHLYFDKAGQKTYERHSLSHFVNLDNLRANNTKGSLPINVI
VFDGKSKCEESSAKSASVYYSQLMCQPILLLDQALVSDVGDSAEVAVKMFDAYVNTFSSTENVPMEKLKTLVATAEA
ELAKNVSLDNVLSTFISAARQGFVDSDVETKDVVECLKLSHQSDIEVTGDSCNNYMLTYNKVENMTPRDLGACIDCS
ARHINAQVAKSHNIALIWNVKDFMSLSEQLRKQIRSAAKKNNLPFKLTCATTRQVVNVVTTKIALKGGKIVNNWLKQ
LIKVTLVFLFVAAIFYLITPVHVMSKHTDFSSEIIGYKAIDGGVTRDIASTDTCFANKHADFDTWFSQRGGSYTNDK
ACPLIAAVITREVGFVVPGLPGTILRTTNGDFLHFLPRVFSAVGNICYTPSKLIEYTDFATSACVLAAECTIFKDAS
GKPVPYCYDTNVLEGSVAYESLRPDTRYVLMDGSIIQFPNTYLEGSVRVVTTFDSEYCRHGTCERSEAGVCVSTSGR
WVLNNDYYRSLPGVFCGVDAVNLLTNMFTPLIQPIGALDISASIVAGGIVAIVVTCLAYYFMRFRRAFGEYSHVVAF
NTLLFLMSFTVLCLTPVYSFLPGVYSVIYLYLTFYLTNDVSFLAHIQWMVMFTPLVPFWITIAYIICISTKHFYWFF
SNYLKRRVVFNGVSFSTFEEAALCTFLLNKEMYLKLRSDVLLPLTQYNRYLALYNKYKYFSGAMDTTSYREAACCHL
AKALNDFSNSGSDVLYQPPQTSITSAVLOSGFRKMAFPSGKVEGCMVQVTCGTTTLNGLWLDDVVYCPRHVICTSED
MLNPNYEDLLIRKSNHNFLVQAGNVOLRVIGHSMONCVLKLKVDTANPKTPKYKFVRIQPGQTFSVLACYNGSPSGV
YQCAMRPNFTIKGSFLNGSCGSVGFNIDYDCVSFCYMHHMELPTGVHAGTDLEGNFYGPFVDRQTAQAAGTDTTITV
NVLAWLYAAVINGDRWFLNRFTTTLNDENLVAMKYNYEPLTQDHVDILGPLSAQTGIAVLDMCASLKELLQNGMNGR
TILGSALLEDEFTPFDVVRQCSGVTFQSAVKRTIKGTHHWLLLTILTSLLVLVQSTQWSLFFFLYENAFLPFAMGII
AMSAFAMMFVKHKHAFLCLFLLPSLATVAYFNMVYMPASWVMRIMTWLDMVDTSLSGFKLKDCVMYASAVVLLILMT
ARTVYDDGARRVWTLMNVLTLVYKVYYGNALDQAISMWALIISVTSNYSGVVTTVMFLARGIVEMCVEYCPIFFITG
NTLQCIMLVYCFLGYFCTCYFGLFCLLNRYFRLTLGVYDYLVSTQEFRYMNSQGLLPPKNSIDAFKLNIKLLGVGGK
PCIKVATVQSKMSDVKCTSVVLLSVLQQLRVESSSKLWAQCVQLHNDILLAKDTTEAFEKMVSLLSVLLSMQGAVDI
NKLCEEMLDNRATLQAIASEFSSLPSYAAFATAQEAYEQAVANGDSEVVLKKLKKSLNVAKSEFDRDAAMQRKLEKM
ADQAMTQMYKQARSEDKRAKVTSAMQTMLFTMLRKLDNDALNNIINNARDGCVPLNIIPLTTAAKLMVVIPDYNTYK
NTCDGTTFTYASALWEIQQVVDADSKIVQLSEISMDNSPNLAWPLIVTALRANSAVKLONNELSPVALROMSCAAGT
TQTACTDDNALAYYNTTKGGRFVLALLSDLQDLKWARFPKSDGTGTIYTELEPPCRFVTDTPKGPKVKYLYFIKGLN
NLNRGMVLGSLAATVRLOAGNATEVPANSTVLSFCAFAVDAAKAYKDYLASGGQPITNCVKMLCTHTGTGQAITVTP
EANMDQESFGGASCCLYCRCHIDHPNPKGFCDLKGKYVQIPTTCANDPVGFTLKNTVCTVCGMWKGYGCSCDQLREP
MLQSADAQSFLNGFAV-
# 5′3′ Frame 2
WRALSLVSTRKHTSNSVCLFYRFATCSYVALETPWRRSYQRHVNILKMALVA--KLKKAFCLNLNSPMCSSNVRMLE
LHLMVMLWLSW-QNSKAFSTVVVVRHLVSLSLMWAKYQWLTARFFFVRTVIKELVAIVTAPI-SHLT-ATSLALILM
KIFKKTGTLNIAVVLPVNSCVSLTEGHTLAMSITTSVALMATLLSALKTF-HVLVKLHALCPNNWTLLTLRGVYTAA
VNMSMKLLGTRNVLKRAMNCRHLLKLNWQRNLTPSMGNVQILYFP-IP-SRLFNQGLKRKSLMALWVEFDLSIQLRH
QMNATKCAFQLS-SVIIVVKLHGRRAILLKPLANFVALRI-LKKVPLLVVTYPKMLLLKFIVQHVTIQK-DLSIVLP
NTIMNLA-KPFFVRVVALLPLEAVCSLMLVAITSVPIGFHVLALT-VVTIQVLLEKVPKVLMTTFLKYSKKRKSTSI
LLVTLNLMKRSPLFWHLFLLPQVLLWKL-KVWIIKHSNKLLNPVVILKLQKEKLKKVPGILVNRNQY-VLFMHLHQR
LLVLYDQFSPALLKLLKILCVFYRRPL-QY-MEFHSIH-DSLML-CSHLIWLLTI-L-WPTLQVVLES-LRSG-LTS
LALFMKNSNPSLIGLKRSLRKV-SFLETVGKLLNLSQPVLVKLSVDKLSPVQRKLRRVFRHSLSL-INFWLCVLTLS
LLVELNLKP-I-VKHLSRTQRDCTESVLNPEKKLAYSCL-KPQKKLSS-REKHFPQKC-QRKLS-KLVIYNH-NNLL
VKLLKLHWLVHQFVLTGLCCSKSKTQKSTVPLHLI-W-QTIPSHSKAVHQQRLLLVMTL--KCKVTRV-ISLLNLMK
GLIKYLMRSALPIQLNSVQK-MSSPVLWQMLS-KLCNQYLNYLHHWALI-MSGVWLHTTYLMSLVSLNWLHICIVLS
TLQMRMKKKVIVKKKSLSHQLNMSMVLKMITKVNLWNLVPLLLLFNLKKSKKKIG-MMIVNKLLVNKTAVRTIRQLL
FKQLLRENLN-RWNLHQLFRLLK-IVLVVI-+NLNLLTMYTLKMQTLWKKLKR-NQQWLLMQPMFTLNMEEVLQEP-
IRLLTMPCKLNLMIT-LLMDHLKWVVVVF-ADTILLNTVFMLSAQMLTKVKTENFLRVLMKILISTKFYLHHYYQLV
FLVLTLYIL-EFV-ILFAQMST-LSLIKISMTNLFQAFWK-RVKSKLNKRSLRFLKRKLSHL-LKVNLQLNRENKMI
RKSKLVLKKLQQLWKKLSSSQKTCYFILTLMAIFIQILPLLLVTLTSLS-RKMLHI-WVMLFKRVF-LLWLYLLKRL
VALLKC-RKL-EKCQQTII-PLTRVRV-MVTL-RRQRQCLKSVKVPFTFYHLLSLMRSKKFLELFLGICEKCLHMQK
KHAN-CLSVWKLKP-FQLYSVNIRVLKYKRVWLIMVLDFTFTPVKQL-RHLSTHLTI-MKLLLQCHLAM-HMA-IWK
KLLGI-DLSKCQLQFLFLHLMLLQRIMVILLLLLKHLKNILLKPSHLLVPIKIGPILDNLHN-V-NFLREVIKVYIT
LVILPHST-MVKLSPLTILRHFFL-EK-GLLRCLQQ-TTLTSTRKLWTCQ-HMDNSLVQLIWMELMLLK-NLIIHMK
VKHFMFYLMMTLYVLRLLSTTTQLILVFWVGTCQH-ITLKSGNTHKLMV-LLLNGQITTVILPLHC-HSNK-S-SLI
HLLYKMLITEQGLVKLLTFVHLS-PTVIRQ-VS-VMLEKQ-VTCFNMPI-ILAKES-TWCVKLVDNSRQPLRV-KLL
CTWAHFLMNNLRKVFRYLVRVVNKLQNI-YNRSHLLL-CQHHLLSMNLSMVHLLVLVSTLVITSVVTINI-LLKKLC
IA-TVLYLQSPQNTKVLLRMFSTKKTVTQQP-NQLLINWMVLFVQKLTLSWTIIIRKTILISQSNQLILYQTNHIQT
QASIILSLYVIISNLLMI-TS-LVIRNLLQESLKLHFSLT-MVMWWLLIINTTHPLLRKELNCYINLLFGMLTMQLI
KPRINQIPGVYVVFGAQNQLKHQIRLMY-SQRTRREWIILPAKI-NQSLKK-WKILPYRKTFLSVM-KLPKL-ETLY
LNQQIIV-KLQKRLATQI-WLLM-TILVLLLRNLMNYLEY-V-KPLLLMV-LLLIVSLGIL-LIMLSLFLTKLLVQL
LT-LHGV-TVFVLIICLISLLYCYNCVLLLEVQILELKHLCRLL-QRILLRVSVNFV-RLHLII-SHLIFLN--IL-
FGFYY-VFA-VL-STQPLL-VF-CLI-ACLLTVLVTEKAI-TLLMSLLQPTVLVLYLVVFVLVV-IL-TPILL-KLY
KLPFHLINGI-LLLA-LQSGFWHIFFSLGFSMYLDWLQSCNCFSAILQYILLVILGLCG--LILYKWPRFQLWLECT
SSLHHFIMYGKVMCML-TVVIHQLV-CVTNVIEQQESNVQLLLMVLEGPFMSMLMEVKAFANYTIGIVLIVIHSVLV
VHLLVMKLRETCHYSLKDQ-ILLTSLLTSLIVLQ-RMVPSIFTLIKLVKRLMKDILSLILLT-TT-ELITLKVHCLL
ML-FLMVNQNVKNHLQNQRLFTTVSLCVNLYCY-IRH-CLMLVIVRKLQLKCLMLTLIRFHQLLTYQWKNSKH-LQL
QKLNLQRMCP-TMSYLLLFQQLGKGLLIQM-KLKMLLNVLNCHINLT-KLLAIVVITICSPITKLKT-HPVTLVLVL
TVVRVILMRR-QKVTTLL-YGTLKISCHCLNNYENKYVVLLKRITYLLS-HVQLLDKLLML-QQR-HLRVVKLLIIG
-SS-LKLHLCSFLLLLFSI--HLFMSCLNILTFQVKS-DTRLLMVVSLVT-HLQILVLLTNMLILTHGLASVVVVIL
MTKLAH-LLQS-QEKWVLSCLVCLARYYAQLMVTFCISYLEFLVQLVTSVTHHQNL-STLTLQHQLVFWLLNVQFLK
MLLVSQYHIVMIPMY-KVLLLMKVYALTHVMCSWMALLENFLTPTLKVLLEW-QLLILSTVGTALVKDQKLVFVYLL
VVDGYLTMIITDLYQEFSVV-ML-IYLLICLHH-FNLLVLWTYQHL--LVVL-LS--HALPTIL-GLEELLVNTVM-
LPLILYYSLCHSLYSV-HQFTHSYLVFILLFTCT-HFILLMMFLF-HIFSGWLCSHL-YLSG-QLLISFVFPQSISI
GSLVIT-RDV-SLMVFPLVLLKKLRCAPFC-IKKCI-SCVVMCYYLLRNIIDT-LFIISTSILVEQWIQLATEKLLV
VISQRLSMTSVTQVLMFFTNHHKPLSPQLFCRVVLEKWHSHLVKLRVVWYK-LVVQLHLTVFGLMT-FTVQDM-SAP
LKTCLTLIMKIYSFVSLIIISWYRLVMENSGLLDILCKIVYLSLRLIQPILRHLSISLEAFNQDRLFQC-LVTMVHH
LVFTNVL-GPISLLRVHSLMVHVVVLVLT-IMTVSLFVTCTIWNYQLEFMLAQT-KVTFMDLLLTGKQHKQLVRTQL
LQLMF-LGCTLLL-METGGFSIDLPQLLMTLTLWL-STIMNL-HKTMLTY-DLFLLKLELPF-ICVLH-KNYCKMV-
MDVPYWVVLY-KMNLHLLMLLDNAQVLLSKVQ-KEQSRVHTTGCYSQF-LHF-F-SRVLNGLCSFFCMKMPFYLLLW
VLLLCLLLQ-CLSNISMHFSVCFCYLLLPL-LILIWSICLLVG-CVL-HGWIWLILVCLVLS-KTVLCMHQL-CY-S
L-QQELCMMMVLGECGHL-MS-HSFIKFIMVML-IKPFPCGLL-SLLLLTTQV-LQLSCFWPEVLFLCVLSIALFSS
-LVIHFSV-C-FIVS-AIFVLVTLASFVYSTATLD-LLVFMIT-FLHRSLDI-IHRDYSHPRIA-MPSNSTLNCWVL
VANLVSK-PLYSLKCQM-SAHQ-SYSQFCNNSE-NHHLNCGLNVSSYTMTFS-LKILLKPLKKWFHYFLFCFPCRVL
-T-TSFVKKCWTTGQPYKL-PQSLVPFHHMQLLLLLKKLMSRLLLMVILKLFLKS-RSL-MWLNLNLTVMQPCNVSW
KRWLIKL-PKCINRLDLRTRGQKLLVLCRQCFSLCLESWIMMHSTTLSTMQEMVVFP-T-YLLQQQPN-WLSYQTIT
HIKIRVMVQHLLMHQHCGKSNRL-MQIVKLENLVKLVWTIHLI-HGLLL-QL-GPILLSNYRIMSLVLLHYDRCLVL
PVLHKLLALMTMR-LTTTQQREVGLYLHCYPIYRI-NGLDSLRVMELVLSIQNWNHLVGLLQTHLKVLK-SIYTLLK
D-TT-IEVWYLVV-LPQYVYKLVMQQKCLPIQLYYLSVLLL-MLLKLTKII-LVGDNQSLIVLRCCVHTLVLVRQ-Q
LHRKPIWIKNPLVVHRVVCTAVAT-IIQILKDFVT-KVSMYKYLQLVLMTLWVLHLKTQSVPSAVCGKVMAVVVINS
ANPCFSQLMHNRF-TGLRC
# 5′3′ Frame 3
GEPCPWFQRENTRPTQFACFTGSRRARTWLWRLRGGGLIRGTSTS-RWHLWLSRS-KRRFAST-TALCVHQTFGCSN
CTSWSCYG-AGSRTRRHSVRS-W-DTWCPCPSCGRNTSGLPQGSSS-ER--RSWWP-LRRRSKVI-LRRRAWH-SL-
RFSRKLEH-T-QWCYP-THA-A-RRGIHSLCR-QLLWP-WLPS-VH-RPSSTCW-SFMHFVRTTGLY-H-EGCILLP
-T-A-NCLVHGTF-KEL-IADTF-N-IGKEI-HLQWGMSKFCISLKFHNQDYSTKG-KEKA-WLYG-NSICLSSCVT
K-MQPNVPFNSHEV-SLW-NFMADGRFC-SHLRILWH-EFD-RRCHYLWLLTPKCCC-NLLSSMSQFRSRT-A-SCR
IP--IWLENHSS-GWSHYCLWRLCVLLCWLP-QVCLLGSTC-R-HRL-PYRCCWRRFRRS--QPS-NTPKRESQHQY
CW-L-T--RDRHYFGIFFCFHKCFCGNCERFGL-SIQTNC-ILW-F-SYKRKS-KRCLEYW-TEINTESSLCICIRG
CSCCTINFLPHS-NCSKFCACFTEGRYNNTRWNFTVFTETH-CYDVHI-FGY-QSSCNGLHYRWCCSVDFAVAN-HL
WHCL-KTQTRP-LA-REV-GRCRVS-RRLGNC-IYLNLCL-NCRWTNCHLCKGN-GECSDIL-ACK-IFGFVC-LYH
YWWS-T-SLEFR-NICHALKGIVQKVC-IQRRNWPTHASKSPKRNYLLRGRNTSHRSVNRGSCLENW-FTTIRTTY-
-SC-SSIGWYTSLY-RAYVARNQRHRKVLCPCT-YDGNKQYLHTQRRCTNKGYFW--HCDRSARLQECEYHF-T--K
D--ST--EVLCLYS-TRYRSK-VRLCCGRCCHKNFATSI-ITYTTGH-FR-VEYGYILLI--VW-V-IGFTYVLFFL
PSR-G-RRR-L-RRRV-AINSI-VWY-R-LPR-TFGIWCHFCCSST-RRARRRLVR---STNCWSTRRQ-GQSDNYY
SNNC-GSTSIRDGTYTSCSDY-SE-F-WLFKTY-QCIH-KCRHCGRS-KGKTNSGC-CSQCLP-TWRRCCRSLK-GY
-QCHAS-I--LHSY-WTT-SGW-LCFKRTQSC-TLSSCCRPKC-QR-RHSTS-ECL-KF-SARSSTCTIIISWYFWC
-PYTFFKSLCRYCSHKCLLSCL--KSL-QTCFKLFGNEE-KAS-TKDR-DS-RGS-AIYN-K-TFS-TEKTR--ENQ
SLC-RSYNNSGRN-VPHRKLVTLY-H-WQSSSRFCHSC--H-HHFLKERCSIYSG-CCSRGCFNCCGYTY-KGWWHY
-NASESFEKSANRQLYNHLPGSGFKWLHCRGGKDSA-KV-KCLLHSTIYYL--EARNSWNCFLEFARNACTCRRNTQ
INACLCGN-SHSFNYTA-I-GY-NTRGCG-LWC-ILLLHQ-NNCSVTYQHT-RSK-NSCYNATWLCNTWLKFGRSCS
VYEISQSASYSFCFFT-CCYSV-WLSYFFF-NT-RTFY-NHLTCWFL-RLVLFWTIYTTRYRIS-ER--KCILH--S
YHIPPRW-SYHL-QS-DTSFFERSEDY-GVYNSRQH-PPHASCGHVNDIWTTVWSNLFGWS-CY-NKTS-FT-R-NI
LCFT---HSTC-GF-VLPHN-S-FSG-VHVSIKSH-KVEIPTS-WFNFY-MGR-QLLSCHCIVNTPTNRVEV-STCS
TRCLLQSKGW-SC-LLCTYLSLL--DSR-VR-C-RNNELLVSTCQFRFLQKSLERGV-NLWTTADNP-GCRSCYVHG
HTFL-TI-ERCSDTLYVW-TSYKISSTTGVTFCYDVSTTCSV-T-AWYIYLC--VHW-LPVWSL-TYNF-RNFVLHR
RCFTYKVLRIQRSYYGCFLQRKQLHNNHKTSYL-IGWCCLYRN-P-VGQLL-ERQFLFHRATN-SCTKPTISKRKLR
-F-VCM--YQIC--FKPVNWL-ETCFKRA-SYIFP-LKW-CGGY-L-TLHTLF-ERS-IVT-TYCLAC-QCN--SHV
-TKYLVYTLSLEHKTS-NIKFV-CTEVRGRAGNG-SCLRRSKTSL-RSSGKSYHTERRS-V-CENYRSCRRHYT-TS
K--FKNYRRGWPHRSNGCLCRQF-SYY-ET--II-SIRFENPCYSWFSCC--CPLGYYS-LC-AFS-QSC-YNY-HS
YTVFKPCLY-LYALFLYFIATIVYFY-KYKF-N-SIYADYYSKEYC-ECR-ILSRGFI-LFEVT-FF-TDKYYNLVF
TIKCLPRFFNLLNRCFRCFNV-FRHAFLLYWLQRRLFELY-CHYCNLLYWFYTL-CLS-WFRFFRHLSFFRNYTNYH
FIF-MGFNCFWLSCRVVFGIYSFH-VFLCTWIGCNHAIVFQLFCSTFY--FLAYVVNN-SCTNGPDFSYG-NVHLLC
IILLCMEKLCACCRRL-FINLYDVLQT--SNKSRMYNYC-WC-KVLLCLC-WR-RLLQTTQLELC-L-YILCW-YIY
---SCERLVTTV-KTNKSY-PVFLHR--CYSEEWFHPSLL--SWSKDL-KTFSLSFC-LRQPES--H-RFIAY-CYS
F-W-IKM-RIICKISVCLLQSAYVSTYTVTRSGISV-CW--CGSCS-NV-CLR-YVFINF-RINGKTQNTSCNCRS-
TCKECVLRQCLIYFYFSSSARVC-FRCRN-RCC-MS-IVTSI-HRSYWR-L--LYAHL-QS-KHDTP-PWCLY-L-C
ASY-CAGSKKSQHCFDMER-RFHVIV-TTTKTNT-CC-KE-LTF-VDMCNY-TSC-CCNNKDST-GW-NC--LVEAV
N-SYTCVPFCCCYFLFNNTCSCHV-TY-LFK-NHRIQGY-WWCHS-HSIYRYLFC-QTC-F-HMV-PAWW-LY--QS
LPIDCCSHNKRSGFCRAWFAWHDITHN-W-LFAFLT-SF-CSW-HLLHTIKTYRVH-LCNISLCFGC-MYNF-RCFW
-ASTILL-YQCTRRFCCL-KFTP-HTLCAHGWLYYSIS-HLP-RFC-SGNNF-F-VL-ARHL-KIRSWCLCIY-W-M
GT-Q-LLQIFTRSFLWCRCCKFTY-YVYTTNSTYWCFGHISIYSSWWYCSYRSNMPCLLFYEV-KSFW-IQSCSCL-
YFTIPYVIHCTLFNTSLLILTWCLFCYLLVLDILSY--CFFFSTYSVDGYVHTFSTFLDNNCLYHLYFHKAFLLVL-
-LPKETCSL-WCFL-YF-RSCAVHLFVK-RNVSKVA--CAITSYAI--ILSSL--VQVF-WSNGYN-LQRSCLLSSR
KGSQ-LQ-LRF-CSLPTTTNLYHLSCFAEWF-KNGIPIW-S-GLYGTSNLWYNYT-RSLA--RSLLSKTCDLHL-RH
A-P-L-RFTHS-V-S-FLGTGW-CSTQGYWTFYAKLCT-A-G-YSQS-DT-V-VCSHSTRTDFFSVSLLQWFTIWCL
PMCYEAQFHY-GFIP-WFMW-CWF-HRL-LCLFLLHAPYGITNWSSCWHRLRR-LLWTFC-QANSTSSWYGHNYYS-
CFSLVVRCCYKWRQVVSQSIYHNS--L-PCGYEVQL-TSNTRPC-HTRTSFCSNWNCRFRYVCFIKRITAKWYEWTY
HIG-CFIRR-IYTF-CC-TMLRCYFPKCSEKNNQGYTPLVVTHNFDFTFSFSPEYSMVFVLFFV-KCLFTFCYGYYC
YVCFCNDVCQT-ACISLFVFVTFSCHCSLF-YGLYAC-LGDAYYDMVGYG-Y-FVWF-AKRLCYVCISCSVTNPYDS
KNCV--WC-ESVDTYECLDTRL-SLLW-CFRSSHFHVGSYNLCYF-LLRCSYNCHVFGQRYCFYVC-VLPYFLHNW-
YTSVYNASLLFLRLFLYLLLWPLLFTQPLL-TDSWCL-LLSFYTGV-IYEFTGTTPTQE-HRCLQTQH-IVGCWWQT
LYQSSHCTV-NVRCKVHISSLTLSFATTQSRIII-IVGSMCPVTQ-HSLS-RYY-SL-KNGFTTFCFAFHAGCCRHK
QAL-RNAGQQGNLTSYSLRV-FPSIICSFCYCSRSL-AGCC-W-F-SCS-KVEEVFECG-I-I-P-CSHAT-VGKDG
-SSYDPNV-TG-I-GQEGKSY-CYADNAFHYA-KVG--CTQQHYQQCKRWLCSLEHNTSYNSSQTNGCHTRL-HI-K
YV-WYNIYLCISIVGNPTGCRCR--NCST--N-YGQFT-FSMASYCNSFKGQFCCQITE--A-SCCTTTDVLCCRYY
TNCLH--QCVSLLQHNKGR-VCTCTVIRFTGFEMG-IP-E-WNWYYLYRTGTTL-VCYRHT-RS-SEVFILY-RIKQ
PK-RYGTW-FSCHSTSTSW-CNRSACQFNCIIFLCFCCRCC-SLQRLSS-WGTTNH-LC-DVVYTHWYWSGNNSYTG
SQYGSRILWWCIVLSVLPLPHRSSKS-RIL-LKR-VCTNTYNLC--PCGFYT-KHSLYRLRYVERLWL-L-STPRTH
ASVS-CTIVEKRVCGV

The nucleic acid sequence of ORF1a); SEQ ID NO:1 6) is:

atggagagccttgtccctggtttcaacgagaaaacacacgtccaactcagtttgcctgttttacaggttcgcgacgt
gctcgtacgtggctttggagactccgtggaggaggtcttatcagaggcacgtcaacatcttaaagatggcacttgtg
gcttagtagaagttgaaaaaggcgttttgcctcaacttgaacagccctatgtgttcatcaaacgttcggatgctcga
actgcacctcatggtcatgttatggttgagctggtagcagaactcgaaggcattcagtacggtcgtagtggtgagac
acttggtgtccttgtccctcatgtgggcgaaataccagtggcttaccgcaaggttcttcttcgtaagaacggtaata
aaggagctggtggccatagttacggcgccgatctaaagtcatttgacttaggcgacgagcttggcactgatccttat
gaagattttcaagaaaactggaacactaaacatagcagtggtgttacccgtgaactcatgcgtgagcttaacggagg
ggcatacactcgctatgtcgataacaacttctgtggccctgatggctaccctcttgagtgcattaaagaccttctag
cacgtgctggtaaagcttcatgcactttgtccgaacaactggactttattgacactaagaggggtgtatactgctgc
cgtgaacatgagcatgaaattgcttggtacacggaacgttctgaaaagagctatgaattgcagacaccttttgaaat
taaattggcaaagaaatttgacaccttcaatggggaatgtccaaattttgtatttcccttaaattccataatcaaga
ctattcaaccaagggttgaaaagaaaaagcttgatggctttatgggtagaattcgatctgtctatccagttgcgtca
ccaaatgaatgcaaccaaatgtgcctttcaactctcatgaagtgtgatcattgtggtgaaacttcatggcagacggg
cgattttgttaaagccacttgcgaattttgtggcactgagaatttgactaaagaaggtgccactacttgtggttact
taccccaaaatgctgttgttaaaatttattgtccagcatgtcacaattcagaagtaggacctgagcatagtcttgcc
gaataccataatgaatctggcttgaaaaccattcttcgtaagggtggtcgcactattgcctttggaggctgtgtgtt
ctcttatgttggttgccataacaagtgtgcctattgggttccacgtgctagcgctaacataggttgtaaccatacag
gtgttgttggagaaggttccgaaggtcttaatgacaaccttcttgaaatactccaaaaagagaaagtcaacatcaat
attgttggtgactttaaacttaatgaagagatcgccattattttggcatctttttctgcttccacaagtgcttttgt
ggaaactgtgaaaggtttggattataaagcattcaaacaaattgttgaatcctgtggtaattttaaagttacaaaag
gaaaagctaaaaaaggtgcctggaatattggtgaacagaaatcaatactgagtcctctttatgcatttgcatcagag
gctgctcgtgttgtacgatcaattttctcccgcactcttgaaactgctcaaaattctgtgcgtgttttacagaaggc
cgctataacaatactagatggaatttcacagtattcactgagactcattgatgctatgatgttcacatctgatttgg
ctactaacaatctagttgtaatggcctacattacaggtggtgttgttcagttgacttcgcagtggctaactaacatc
tttggcactgtttatgaaaaactcaaacccgtccttgattggcttgaagagaagtttaaggaaggtgtagagtttct
tagagacggttgggaaattgttaaatttatctcaacctgtgcttgtgaaattgtcggtggacaaattgtcacctgtg
caaaggaaattaaggagagtgttcagacattctttaagcttgtaaataaatttttggctttgtgtgctgactctatc
attattggtggagctaaacttaaagccttgaatttaggtgaaacatttgtcacgcactcaaagggattgtacagaaa
gtgtgttaaatccagagaagaaactggcctactcatgcctctaaaagccccaaaagaaattatcttcttagagggag
aaacacttcccacagaagtgttaacagaggaagttgtcttgaaaactggtgatttacaaccattagaacaacctact
agtgaagctgttgaagctccattggttggtacaccagtttgtattaacgggcttatgttgctcgaaatcaaagacac
agaaaagtactgtgcccttgcacctaatatgatggtaacaaacaataccttcacactcaaaggcggtgcaccaacaa
aggttacttttggtgatgacactgtgatagaagtgcaaggttacaagagtgtgaatatcacttttgaacttgatgaa
aggattgataaagtacttaatgagaagtgctctgcctatacagttgaactcggtacagaagtaaatgagttcgcctg
tgttgtggcagatgctgtcataaaaactttgcaaccagtatctgaattacttacaccactgggcattgatttagatg
agtggagtatggctacatactacttatttgatgagtctggtgagtttaaattggcttcacatatgtattgttctttc
taccctccagatgaggatgaagaagaaggtgattgtgaagaagaagagtttgagccatcaactcaatatgagtatgg
tactgaagatgattaccaaggtaaacctttggaatttggtgccacttctgctgctcttcaacctgaagaagagcaag
aagaagattggttagatgatgatagtcaacaaactgttggtcaacaagacggcagtgaggacaatcagacaactact
attcaaacaattgttgaggttcaacctcaattagagatggaacttacaccagttgttcagactattgaagtgaatag
ttttagtggttatttaaaacttactgacaatgtatacattaaaaatgcagacattgtggaagaagctaaaaaggtaa
aaccaacagtggttgttaatgcagccaatgtttaccttaaacatggaggaggtgttgcaggagccttaaataaggct
actaacaatgccatgcaagttgaatctgatgattacatagctactaatggaccacttaaagtgggtggtagttgtgt
tttaagcggacacaatcttgctaaacactgtcttcatgttgtcggcccaaatgttaacaaaggtgaagacattcaac
ttcttaagagtgcttatgaaaattttaatcagcacgaagttctacttgcaccattattatcagctggtatttttggt
gctgaccctatacattctttaagagtttgtgtagatactgttcgcacaaatgtctacttagctgtctttgataaaaa
tctctatgacaaacttgtttcaagctttttggaaatgaagagtgaaaagcaagttgaacaaaagatcgctgagattc
ctaaagaggaagttaagccatttataactgaaagtaaaccttcagttgaacagagaaaacaagatgataagaaaatc
aaagcttgtgttgaagaagttacaacaactctggaagaaactaagttcctcacagaaaacttgttactttatattga
cattaatggcaatcttcatccagattctgccactcttgttagtgacattgacatcactttcttaaagaaagatgctc
catatatagtgggtgatgttgttcaagagggtgttttaactgctgtggttatacctactaaaaaggctggtggcact
actgaaatgctagcgaaagctttgagaaaagtgccaacagacaattatataaccacttacccgggtcagggtttaaa
tggttacactgtagaggaggcaaagacagtgcttaaaaagtgtaaaagtgccttttacattctaccatctattatct
ctaatgagaagcaagaaattcttggaactgtttcttggaatttgcgagaaatgcttgcacatgcagaagaaacacgc
aaattaatgcctgtctgtgtggaaactaaagccatagtttcaactatacagcgtaaatataagggtattaaaataca
agagggtgtggttgattatggtgctagattttacttttacaccagtaaaacaactgtagcgtcacttatcaacacac
ttaacgatctaaatgaaactcttgttacaatgccacttggctatgtaacacatggcttaaatttggaagaagctgct
cggtatatgagatctctcaaagtgccagctacagtttctgtttcttcacctgatgctgttacagcgtataatggtta
tcttacttcttcttctaaaacacctgaagaacattttattgaaaccatctcacttgctggttcctataaagattggt
cctattctggacaatctacacaactaggtatagaatttcttaagagaggtgataaaagtgtatattacactagtaat
cctaccacattccacctagatggtgaagttatcacctttgacaatcttaagacacttctttctttgagagaagtgag
gactattaaggtgtttacaacagtagacaacattaacctccacacgcaagttgtggacatgtcaatgacatatggac
aacagtttggtccaacttatttggatggagctgatgttactaaaataaaacctcataattcacatgaaggtaaaaca
ttttatgttttacctaatgatgacactctacgtgttgaggcttttgagtactaccacacaactgatcctagttttct
gggtaggtacatgtcagcattaaatcacactaaaaagtggaaatacccacaagttaatggtttaacttctattaaat
gggcagataacaactgttatcttgccactgcattgttaacactccaacaaatagagttgaagtttaatccacctgct
ctacaagatgcttattacagagcaagggctggtgaagctgctaacttttgtgcacttatcttagcctactgtaataa
gacagtaggtgagttaggtgatgttagagaaacaatgagttacttgtttcaacatgccaatttagattcttgcaaaa
gagtcttgaacgtggtgtgtaaaacttgtggacaacagcagacaacccttaagggtgtagaagctgttatgtacatg
ggcacactttcttatgaacaatttaagaaaggtgttcagataccttgtacgtgtggtaaacaagctacaaaatatct
agtacaacaggagtcaccttttgttatgatgtcagcaccacctgctcagtatgaacttaagcatggtacatttactt
gtgctagtgagtacactggtaattaccagtgtggtcactataaacatataacttctaaagaaactttgtattgcata
gacggtgctttacttacaaagtcctcagaatacaaaggtcctattacggatgttttctacaaagaaaacagttacac
aacaaccataaaaccagttacttataaattggatggtgttgtttgtacagaaattgaccctaagttggacaattatt
ataagaaagacaattcttatttcacagagcaaccaattgatcttgtaccaaaccaaccatatccaaacgcaagcttc
gataattttaagtttgtatgtgataatatcaaatttgctgatgatttaaaccagttaactggttataagaaacctgc
ttcaagagagcttaaagttacatttttccctgacttaaatggtgatgtggtggctattgattataaacactacacac
cctcttttaagaaaggagctaaattgttacataaacctattgtttggcatgttaacaatgcaactaataaagccacg
tataaaccaaatacctggtgtatacgttgtctttggagcacaaaaccagttgaaacatcaaattcgtttgatgtact
gaagtcagaggacgcgcagggaatggataatcttgcctgcgaagatctaaaaccagtctctgaagaagtagtggaaa
atcctaccatacagaaagacgttcttgagtgtaatgtgaaaactaccgaagttgtaggagacattatacttaaacca
gcaaataatagtttaaaaattacagaagaggttggccacacagatctaatggctgcttatgtagacaattctagtct
tactattaagaaacctaatgaattatctagagtattaggtttgaaaacccttgctactcatggtttagctgctgtta
atagtgtcccttgggatactatagctaattatgctaagccttttcttaacaaagttgttagtacaactactaacata
gttacacggtgtttaaaccgtgtttgtactaattatatgccttatttctttactttattgctacaattgtgtacttt
tactagaagtacaaattctagaattaaagcatctatgccgactactatagcaaagaatactgttaagagtgtcggta
aattttgtctagaggcttcatttaattatttgaagtcacctaatttttctaaactgataaatattataatttggttt
ttactattaagtgtttgcctaggttctttaatctactcaaccgctgctttaggtgttttaatgtctaatttaggcat
gccttcttactgtactggttacagagaaggctatttgaactctactaatgtcactattgcaacctactgtactggtt
ctataccttgtagtgtttgtcttagtggtttagattctttagacacctatccttctttagaaactatacaaattacc
atttcatcttttaaatgggatttaactgcttttggcttagttgcagagtggtttttggcatatattcttttcactag
gtttttctatgtacttggattggctgcaatcatgcaattgtttttcagctattttgcagtacattttattagtaatt
cttggcttatgtggttaataattaatcttgtacaaatggccccgatttcagctatggttagaatgtacatcttcttt
gcatcattttattatgtatggaaaagttatgtgcatgttgtagacggttgtaattcatcaacttgtatgatgtgtta
caaacgtaatagagcaacaagagtcgaatgtacaactattgttaatggtgttagaaggtccttttatgtctatgcta
atggaggtaaaggcttttgcaaactacacaattggaattgtgttaattgtgatacattctgtgctggtagtacattt
attagtgatgaagttgcgagagacttgtcactacagtttaaaagaccaataaatcctactgaccagtcttcttacat
cgttgatagtgttacagtgaagaatggttccatccatctttactttgataaagctggtcaaaagacttatgaaagac
attctctctctcattttgttaacttagacaacctgagagctaataacactaaaggttcattgcctattaatgttata
gtttttgatggtaaatcaaaatgtgaagaatcatctgcaaaatcagcgtctgtttactacagtcagcttatgtgtca
acctatactgttactagatcaggcattagtgtctgatgttggtgatagtgcggaagttgcagttaaaatgtttgatg
cttacgttaatacgttttcatcaacttttaacgtaccaatggaaaaactcaaaacactagttgcaactgcagaagct
gaacttgcaaagaatgtgtccttagacaatgtcttatctacttttatttcagcagctcggcaagggtttgttgattc
agatgtagaaactaaagatgttgttgaatgtcttaaattgtcacatcaatctgacatagaagttactggcgatagtt
gtaataactatatgctcacctataacaaagttgaaaacatgacaccccgtgaccttggtgcttgtattgactgtagt
gcgcgtcatattaatgcgcaggtagcaaaaagtcacaacattgctttgatatggaacgttaaagatttcatgtcatt
gtctgaacaactacgaaaacaaatacgtagtgctgctaaaaagaataacttaccttttaagttgacatgtgcaacta
ctagacaagttgttaatgttgtaacaacaaagatagcacttaagggtggtaaaattgttaataattggttgaagcag
ttaattaaagttacacttgtgttcctttttgttgctgctattttctatttaataacacctgttcatgtcatgtctaa
acatactgacttttcaagtgaaatcataggatacaaggctattgatggtggtgtcactcgtgacatagcatctacag
atacttgttttgctaacaaacatgctgattttgacacatggtttagccagcgtggtggtagttatactaatgacaaa
gcttgcccattgattgctgcagtcataacaagagaagtgggttttgtcgtgcctggtttgcctggcacgatattacg
cacaactaatggtgactttttgcatttcttacctagagtttttagtgcagttggtaacatctgttacacaccatcaa
aacttatagagtacactgactttgcaacatcagcttgtgttttggctgctgaatgtacaatttttaaagatgcttct
ggtaagccagtaccatattgttatgataccaatgtactagaaggttctgttgcttatgaaagtttacgccctgacac
acgttatgtgctcatggatggctctattattcaatttcctaacacctaccttgaaggttctgttagagtggtaacaa
cttttgattctgagtactgtaggcacggcacttgtgaaagatcagaagctggtgtttgtgtatctactagtggtaga
tgggtacttaacaatgattattacagatctttaccaggagttttctgtggtgtagatgctgtaaatttacttactaa
tatgtttacaccactaattcaacctattggtgctttggacatatcagcatctatagtagctggtggtattgtagcta
tcgtagtaacatgccttgcctactattttatgaggtttagaagagcttttggtgaatacagtcatgtagttgccttt
aatactttactattccttatgtcattcactgtactctgtttaacaccagtttactcattcttacctggtgtttattc
tgttatttacttgtacttgacattttatcttactaatgatgtttcttttttagcacatattcagtggatggttatgt
tcacacctttagtacctttctggataacaattgcttatatcatttgtatttccacaaagcatttctattggttcttt
agtaattacctaaagagacgtgtagtctttaatggtgtttcctttagtacttttgaagaagctgcgctgtgcacctt
tttgttaaataaagaaatgtatctaaagttgcgtagtgatgtgctattacctcttacgcaatataatagatacttag
ctctttataataagtacaagtattttagtggagcaatggatacaactagctacagagaagctgcttgttgtcatctc
gcaaaggctctcaatgacttcagtaactcaggttctgatgttctttaccaaccaccacaaacctctatcacctcagc
tgttttgcagagtggttttagaaaaatggcattcccatctggtaaagttgagggttgtatggtacaagtaacttgtg
gtacaactacacttaacggtctttggcttgatgacgtagtttactgtccaagacatgtgatctgcacctctgaagac
atgcttaaccctaattatgaagatttactcattcgtaagtctaatcataatttcttggtacaggctggtaatgttca
actcagggttattggacattctatgcaaaattgtgtacttaagcttaaggttgatacagccaatcctaagacaccta
agtataagtttgttcgcattcaaccaggacagactttttcagtgttagcttgttacaatggttcaccatctggtgtt
taccaatgtgctatgaggcccaatttcactattaagggttcattccttaatggttcatgtggtagtgttggttttaa
catagattatgactgtgtctctttttgttacatgcaccatatggaattaccaactggagttcatgctggcacagact
tagaaggtaacttttatggaccttttgttgacaggcaaacagcacaagcagctggtacggacacaactattacagtt
aatgttttagcttggttgtacgctgctgttataaatggagacaggtggtttctcaatcgatttaccacaactcttaa
tgactttaaccttgtggctatgaagtacaattatgaacctctaacacaagaccatgttgacatactaggacctcttt
ctgctcaaactggaattgccgttttagatatgtgtgcttcattaaaagaattactgcaaaatggtatgaatggacgt
accatattgggtagtgctttattagaagatgaatttacaccttttgatgttgttagacaatgctcaggtgttacttt
ccaaagtgcagtgaaaagaacaatcaagggtacacaccactggttgttactcacaattttgacttcacttttagttt
tagtccagagtactcaatggtctttgttcttttttttgtatgaaaatgcctttttaccttttgctatgggtattatt
gctatgtctgcttttgcaatgatgtttgtcaaacataagcatgcatttctctgtttgtttttgttaccttctcttgc
cactgtagcttattttaatatggtctatatgcctgctagttgggtgatgcgtattatgacatggttggatatggttg
atactagtttgtctggttttaagctaaaagactgtgttatgtatgcatcagctgtagtgttactaatccttatgaca
gcaagaactgtgtatgatgatggtgctaggagagtgtggacacttatgaatgtcttgacactcgtttataaagttta
ttatggtaatgctttagatcaagccatttccatgtgggctcttataatctctgttacttctaactactcaggtgtag
ttacaactgtcatgtttttggccagaggtattgtttttatgtgtgttgagtattgccctattttcttcataactggt
aatacacttcagtgtataatgctagtttattgtttcttaggctatttttgtacttgttactttggcctcttttgttt
actcaaccgctactttagactgactcttggtgtttatgattacttagtttctacacaggagtttagatatatgaatt
cacagggactactcccacccaagaatagcatagatgccttcaaactcaacattaaattgttgggtgttggtggcaaa
ccttgtatcaaagtagccactgtacagtctaaaatgtcagatgtaaagtgcacatcagtagtcttactctcagtttt
gcaacaactcagagtagaatcatcatctaaattgtgggctcaatgtgtccagttacacaatgacattctcttagcta
aagatactactgaagcctttgaaaaaatggtttcactactttctgttttgctttccatgcagggtgctgtagacata
aacaagctttgtgaagaaatgctggacaacagggcaaccttacaagctatagcctcagagtttagttcccttccatc
atatgcagcttttgctactgctcaagaagcttatgagcaggctgttgctaatggtgattctgaagttgttcttaaaa
agttgaagaagtctttgaatgtggctaaatctgaatttgaccgtgatgcagccatgcaacgtaagttggaaaagatg
gctgatcaagctatgacccaaatgtataaacaggctagatctgaggacaagagggcaaaagttactagtgctatgca
gacaatgcttttcactatgcttagaaagttggataatgatgcactcaacaacattatcaacaatgcaagagatggtt
gtgttcccttgaacataatacctcttacaacagcagccaaactaatggttgtcataccagactataacacatataaa
aatacgtgtgatggtacaacatttacttatgcatcagcattgtgggaaatccaacaggttgtagatgcagatagtaa
aattgttcaacttagtgaaattagtatggacaattcacctaatttagcatggcctcttattgtaacagctttaaggg
ccaattctgctgtcaaattacagaataatgagcttagtcctgttgcactacgacagatgtcttgtgctgccggtact
acacaaactgcttgcactgatgacaatgcgttagcttactacaacacaacaaagggaggtaggtttgtacttgcact
gttatccgatttacaggatttgaaatgggctagattccctaagagtgatggaactggtactatctatacagaactgg
aaccaccttgtaggtttgttacagacacacctaaaggtcctaaagtgaagtatttatactttattaaaggattaaac
aacctaaatagaggtatggtacttggtagtttagctgccacagtacgtctacaagctggtaatgcaacagaagtgcc
tgccaattcaactgtattatctttctgtgcttttgctgtagatgctgctaaagcttacaaagattatctagctagtg
ggggacaaccaatcactaattgtgttaagatgttgtgtacacacactggtactggtcaggcaataacagttacaccg
gaagccaatatggatcaagaatcctttggtggtgcatcgtgttgtctgtactgccgttgccacatagatcatccaaa
tcctaaaggattttgtgacttaaaaggtaagtatgtacaaatacctacaacttgtgctaatgaccctgtgggtttta
cacttaaaaacacagtctgtaccgtctgcggtatgtggaaaggttatggctgtagttgtgatcaactccgcgaaccc
atgcttcagtcagctgatgcacaatcgtttttaaacgggtttgcggtgtaa

As demonstrated in the accompanying examples, the sensing device presently disclosed is capable of detecting various biomarkers in a fluid sample, even when the biomarkers are present in very low concentrations. In particular, detection limits of 1-10 μg/ml of IgG and IgM antibodies can be achieved with millimeter scale electrode dimensions; both detection limits and sensitivities can be improved by several orders of magnitude by changes in the thickness and lateral dimensions of the electrode and thickness of the vibrating substrate.

### Electrodes

In embodiments, the electrode layer comprises at least one working electrode. The electrode lay can comprise working electrode, auxiliary or counter electrode, a reference electrode, or any combination thereof. In embodiments, any one or more of the reference electrode, counter electrode, and working electrode, can be formed out of a suitable electrically conductive material. The electrically conductive material can comprise titanium, titanium nitride, iridium oxide, platinum, gold, aluminum, stainless steel, indium tin ox, silver, mercury, platinum, ruthenium, rhodium, or a combination thereof. In certain embodiments, the conductive material comprises a conductive polymer. Exemplary conductive polymers include polyethylenedioxythiophene (PEDOT), polypyrrole, polyaniline, carbon-nanotube infused poly-dimethylsiloxane (PDMS) or a combination thereof. The electrode layer is coated with micro-porous platinum, nano-porous platinum, nano-gold, or a combination thereof. In embodiments, the particular form, function, and number of electrodes will vary depending on the specific application, the disease or condition-of-interest, the biomarker, the type of sensing device, or a combination thereof.

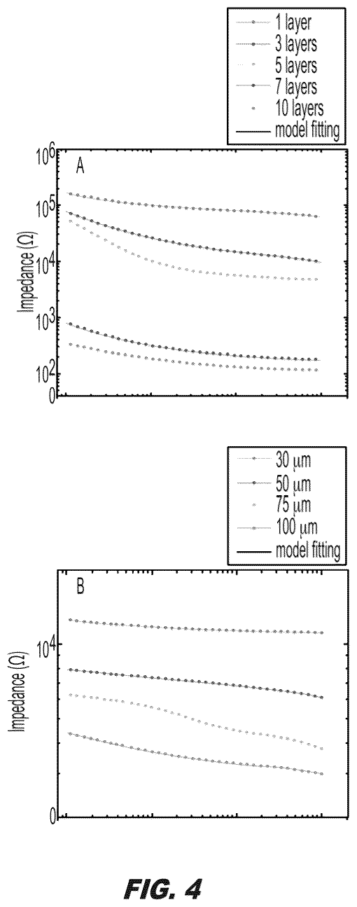

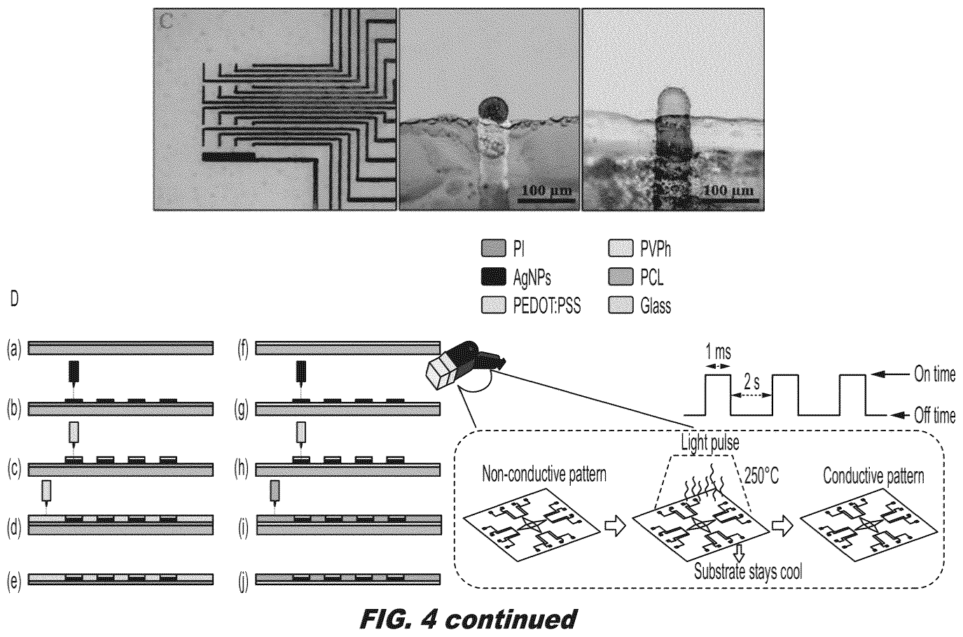

The electrodes can be constructed, patterned, or coated via any of method known in the art. By way of example and as illustrated in FIG. 4, one embodiment comprises the use of inkjet techniques to pattern the electrode layer on the surface of a quartz crystal.

Application of the coating over the electrode can be carried out by first forming a mixture of the component ingredients, which are dissolved in a suitable solvent such as THE, and then applying the dissolved solution using any of a variety of means including, without limitation, spray-coating, spin-coating, dip-coating, roller-coating, blade-coating, etc. The particular choice of coating technique will depend on its compatibility with the structure of the electrochemical cell that forms part of the sensing device of the present invention. During and subsequent to application the solvent used to disperse the components is removed, leaving the coating applied to a surface of the electrode(s).

In embodiments, the physical parameters of the electrodes can vary with fluid the sample volumes, and the biomarker in question. In embodiments, at least one electrode comprises a diameter of at least about 600 nm. At least one electrode can have a diameter or up to about 10 mm. Embodiments can comprise an electrode with a diameter that is greater than about 10 mm. In one embodiment, an electrode has a diameter of up to 30 mm. In certain embodiments the diameter of at least one electrode is less than about 600 nm.

In embodiments, at least one electrode comprises a thickness of at least about 10 nm. At least one electrode can have a thickness of up to about 500 nm. Embodiments can comprise an electrode thickness of greater than about 500 nm.

In certain embodiments, the at least one working electrode is greater than about 1 nm thick. The at least one working electrode can comprise a thickness of between about 1 nm and 500 nm, inclusive. The working electrode can be greater about 25 nm, about 50 nm, about 75 nm, about 100 nm, about 125 nm, about 150 nm, about 175 nm, about 200 nm, about 225 nm, about 250 nm, about 275 nm, about 300 nm, about 325 nm, about 350 nm, about 375 nm, about 400 nm, about 425 nm, about 450 nm, about 475 nm, or about 500 nm. The at least one working electrode can comprise a diameter of at least 10 μm. In embodiments, the at least one working electrode comprises a diameter of up to 150 mm. the diameter of the at least one working electrodes can be about 25 μm, about 50 μm, about 100 μm, about 125 μm, about 150 μm, about 175 μm, about 200 μm, about 225 μm, about 250 μm, about 275 μm, about 300 μm, about 325 μm, about 350 μm, about 375 μm, about 400 μm, about 425 μm, about 450 μm, about 475 μm, about 500 μm, about 525 μm, about 550 μm, about 575 μm, about 600 μm, about 625 μm, about 650 μm, about 675 μm, about 700 μm, about 725 μm, about 750 μm, about 775 μm, about 800 μm, about 825 μm, about 850 μm, about 875 μm, about 900 μm, about 925 μm, about 950 μm, about 975 μm, about 1000 μm., about 1025 μm, about 1050 μm, about 1075 μm, about 1200 μm, about 1225 μm, about 1250 μm, about 1275 μm, about 1300 μm, about 1325 μm, about 1350 μm, about 1375 μm, about 1400 μm, about 1425 μm, about 1450 μm, about 1475 μm, about 1500 μm. In certain embodiments, the at least one working electrode comprises a diameter of greater than about 1 mm, about 10 mm, about 20 mm, about 30 mm, about 40 mm, about 50 mm, about 60 mm, about 70 mm, about 80 mm, about 90 mm, about 100 mm, about 110 mm, about 120 mm, about 130 mm, about 140 mm, or about 150 mm. The at least one working electrode can comprise a diameter of greater than about 150 mm.

The piezoelectric material can comprise a diameter of at least about 1 mm. In embodiments, the piezoelectric material comprises a diameter of up to about 200 mm. The diameter of the piezoelectric material can be up to about 190, up to about 180, up to about 170, up to about 160, up to about 150 mm, up to about 140 mm, up to about 130 mm, up to about 120 mm, up to about 110 mm, or up to about 100 mm. In certain embodiments, the diameter of the piezoelectric material is less than about 1 mm. The diameter of the piezoelectric material can be at least about 1 mm, at least about 2 mm, at least about 3 mm, at least about 4 mm, at least about 5 mm, at least about 6 mm, at least about 7 mm, at least about 8 mm, at least about 9 mm, or at least about 10 mm. In certain embodiments, the piezoelectric material can comprise a diameter of at least about 10 mm, at least about 15 mm, at least about 20 mm, at least about 25 mm, at least about 30 mm, at least about 35 mm, at least about 40 mm, at least about 45 mm, at least about 50 mm, at least about 55 mm, at least about 60 mm, at least about 65 mm, at least about 70 mm, at least about 75 mm, at least about 80 mm, at least about 85 mm, at least about 90 mm, at least about 95 mm, or at least about 100 mm.

In certain embodiments, the piezoelectric material comprises a thickness of at least about 10 μm. The piezoelectric material can comprise a thickness of up to about 5 mm. the thickness of the piezoelectric material can comprise up to about 1 mm, up to about 2 mm, up to about 3 mm, up to about 4 mm, or up to about 5 mm. In embodiments, the thickness of the piezoelectric material can be at least about 1 μm, at least about 2 μm, at least about 3 μm, at least about 4 μm, at least about 5 μm, at least about 6 μm, at least about 7 μm, at least about 8 μm, at least about 9 μm, or at least about 10 μm.

The embodiments disclosed herein are exemplary—the electrodes and piezoelectric material can have varying dimensions to provide for preferred sensitivities

### Stabilizers

In embodiments, the diagnostic platform disclosed herein can be shelf stable at room temperature for up to at least 18 months. In certain embodiments, the diagnostic platform is shelf stable for at least 12 months.

In embodiments, the diagnostic platform comprises at least one stabilizing solution. Non-limiting examples of such stabilizers comprise sucrose, LB Medium and Blocking buffer, Lipidure, glycerin.

In embodiments, the sensing cartridge is stabilized by via stabilizing the surface bound S protein and IgG/IgM antibodies.

In embodiments, the process for adding the stabilizers comprises covering the prepared sensor surface with 1) blocking buffer (Thermo Fisher Scientific, USA) 0.15 M. 2) (5% w/v) sucrose (Acros). 3) coating stabilizer and blocking buffer diluted 1:1 with water. The devices can then be aspirated and dried. All devices can be stored at 50° C. to age the coating (1 day at 50° C. is equal to about 6.5 days at room temperature). We can assess the stability of the coating at 10 days, 3 weeks, 3 months & 6 months [27]. Without being bound by theory, embodiments can comprise an 18-month shelf life. Besides the in-use stability assessment of reagents in the above experiments, the stability of reagents can also be assessed under the following conditions:

Shelf-life stability-Reagent shelf life can be assessed by real-time stability testing, with reagents stored at the specified storage temperature.

Stress testing—the reagents can be cycled through a temperature of 4° C. and ambient temperature to mimic shipping conditions. A separate group of reagents can be cycled through light conditions to mimic shipping conditions. They can then be placed under normal storage conditions and their in-use stability assessed.

As discussed herein the presently disclosed diagnostic platform can obtain data regarding the presence or absence of certain biomarkers that identify a particular condition, disease, or disorder. In embodiments, the sensing device can be communicatively linked to a mobile device application running on a processor of a mobile computing device. In embodiments, the sensing device is communicatively coupled with the mobile device application through one or more wireless communications protocols.

### Transmission/Communication of Data

In embodiments, the mobile device application can present collected data directly to a user through a smartphone application display. The mobile device can be communicatively coupled to one or more applications running on at least one processor of a remote server. The mobile device application can transmit data or results of data analysis (as described below) to the remote server. Third party clients such as health care professionals or researchers can (with appropriate consents and compliance with HIPPA and other federal and state privacy legislation) receive application data through requests to remote server applications. In embodiments, such third party health care provider can then recommend or prescribe a therapeutic agent or other treatment option as appropriate according to the data received.

As indicated above, sensor data can be communicated to a mobile device application which can then be presented to a user, a health professional, a researcher, or a combination thereof. However, the mobile device application can initially analyze and interpret raw sensor data to determine the user's likelihood of having a particular condition, disease, or disorder. The application can then send analytical results to remote server applications for third party access. Under an embodiment, data analytics can be performed by the mobile device application, remote server applications, or a combination thereof.

In embodiments, the diagnostic platform can comprise a microcontroller. The microcontroller monitors and receives data from transducers/sensors. In embodiments, the microcontroller can also send and receive data to memory. Under an embodiment, the microcontroller transmits transducer/sensor data through a transceiver to a mobile device application running on a processor of a smartphone.

As indicated above, the sensor device can be communicatively coupled with the mobile device application through one or more wireless communications protocols. The communicative coupling can comprise a wireless local area network (WLAN). A WLAN connection can implement WiFi™ communications protocols. Alternatively, the communicative coupling can comprise a wireless personal area network WPAN. A WPAN connection can implement Bluetooth™ communications protocols.

Under an embodiment, the sensing device can additionally comprise a data port for relaying data from the sensor device to the mobile device application or other computing device. The data port can comprise a USB connection or any other type of data port. In embodiments, the data port allows for a wired communication between the sensing device and separate computing devices. The data port can be used alone or in combination with the wireless transceiver of the sensing device described above.

In certain embodiments, the sensing device and mobile application are communicatively couple through a local router. The router can comprise a component of the WLAN or WPAN. The router can further communicate with wide area networks, metropolitan area networks, and/or other private/public networks and communication services providing general internet connectivity. Accordingly, in certain embodiments, the sensing device and/or mobile device application can send data to remote server applications. As indicated above, a remote server application can receive raw data from a sensing device and/or mobile device application. Alternatively, the remote server can receive data analytics performed by the mobile device application. Under an embodiment, the remote server application can offer access to third parties and/or systems, i.e., health care practitioners, electronic health records systems, or researchers (assuming consents and compliance with HIPPA and other state/federal privacy legislation). Under an embodiment, a user can access the remote server application and retrieve/review raw data and data analytics using a desktop HTML client application.

In embodiments, a mobile computing device can be configured to receive data from the sensing device. The mobile computing device can comprise a portable digital assistant, a tablet, a smartphone (such as iOS™ and Android™ based devices), a laptop, or similar computing device. The mobile computing device can comprise a wearable device. Under an alternative embodiment, a wearable device can represent an additional component of the network. In other words, an additional wearable device can couple with the communications network and communicate with the sensing device, mobile computing device, and local router. A wearable device application can comprise some or all functionality presented by the mobile device application.

Upon receipt of the data, a mobile device application can relay the data directly to the user, the health professional, a researcher, or a combination thereof. The application can, alternatively or in combination, relay the data to be included within the patient's electronic medical records (EMR) for later diagnosis or evaluation.

In one embodiment, the mobile device application compares the data received to historical patient data. The historical patient data can include data reported through prior scientific publications, data compiled from the health professional's own patient records, data from EMR systems that represent diverse patient populations, or any other source of patient data or combinations thereof. Of course, all such data comply with HIPPA and state/federal data privacy legislation.

Using such information, the application can detect similarities and differences in the patient's data with historical patient data and uses statistical methods to calculate the probability that the user is suffering from a particular condition, disease, or disorder. Using this probability data, a health care professional can make certain recommendations or prescriptions to treat or otherwise alleviate the condition, disease, or disorder or any symptoms associated therewith.

Data can be reported to users via a dashboard type view for the user to see results and receive recommendations. These results are under an embodiment viewable through the application interface. As indicated above, data and data analysis can additionally reside on a remote server. A user can access such data and analysis through a desktop client.

Computer networks suitable for use with the embodiments described herein include local area networks (LAN), wide area networks (WAN), Internet, or other connection services and network variations such as the world wide web, the public internet, a private internet, a private computer network, a public network, a mobile network, a cellular network, a value-added network, and the like. Computing devices coupled or connected to the network can be any microprocessor-controlled device that permits access to the network, including terminal devices, such as personal computers, workstations, servers, mini-computers, main-frame computers, laptop computers, mobile computers, palm top computers, hand-held computers, mobile phones, TV set-top boxes, or combinations thereof. The computer network can include one of more LANs, WANs, Internets, and computers. In embodiments, the computers serve as servers, clients, or a combination thereof.

The systems and methods of the presently disclosed diagnostic platform can be a component of a single system, multiple systems, and/or geographically separate systems. The systems and methods of the presently disclosed diagnostic platform can also be a subcomponent or subsystem of a single system, multiple systems, and/or geographically separate systems. The components of systems and methods of the presently disclosed diagnostic platform can be coupled to one or more other components (not shown) of a host system or a system coupled to the host system.

In certain embodiments, one or more components of the systems and methods of the presently disclosed diagnostic platform and/or a corresponding interface, system or application to which the systems and methods are coupled or connected includes and/or runs under and/or in association with a processing system. The processing system can include any collection of processor-based devices or computing devices operating together, or components of processing systems or devices, as is known in the art. For example, the processing system can include one or more of a portable computer, portable communication device operating in a communication network, and/or a network server. The portable computer can be any of a number and/or combination of devices selected from among personal computers, personal digital assistants, portable computing devices, and portable communication devices, but is not so limited. The processing system can include components within a larger computer system.

The processing system of an embodiment includes at least one processor and at least one memory device or subsystem. The processing system can also include or be coupled to at least one database. The term “processor” as generally used herein can refer to any logic processing unit, such as one or more central processing units (CPUs), digital signal processors (DSPs), application-specific integrated circuits (ASIC), etc. The processor and memory can be monolithically integrated onto a single chip, distributed among a number of chips or components, and/or provided by some combination of algorithms. The methods described herein can be implemented in one or more of software algorithm(s), programs, firmware, hardware, components, circuitry, in any combination.

The components of any system that include the systems and methods of the presently disclosed diagnostic platform can be located together or in separate locations. Communication paths couple the components and include any medium for communicating or transferring files among the components. The communication paths can include wireless connections, wired connections, and hybrid wireless/wired connections. The communication paths also include couplings or connections to networks including local area networks (LANs), metropolitan area networks (MANs), wide area networks (WANs), proprietary networks, interoffice or backend networks, and the Internet. Furthermore, the communication paths include removable fixed mediums like floppy disks, hard disk drives, and CD-ROM disks, as well as flash RAM, Universal Serial Bus (USB) connections, RS-232 connections, telephone lines, buses, and electronic mail messages.

Aspects of the systems and methods of the presently disclosed diagnostic platform described herein can be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the systems and methods of the presently disclosed diagnostic platform can include: microcontrollers with memory (such as electronically erasable programmable read only memory (EEPROM)), embedded microprocessors, firmware, software, etc. Furthermore, aspects of the systems and methods of the presently disclosed diagnostic platform can be embodied in microprocessors having software-based circuit emulation, discrete logic (sequential and combinatorial), custom devices, fuzzy (neural) logic, quantum devices, and hybrids of any of the above device types. The underlying device technologies can be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

It should be noted that any system, method, and/or other components disclosed herein can be described using computer aided design tools and expressed (or represented), as data and/or instructions embodied in various computer-readable media, in terms of their behavioral, register transfer, logic component, transistor, layout geometries, and/or other characteristics. Computer-readable media in which such formatted data and/or instructions can be embodied include, but are not limited to, non-volatile storage media in various forms (e.g., optical, magnetic or semiconductor storage media) and carrier waves that can be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, etc.). When received within a computer system via one or more computer-readable media, such data and/or instruction-based expressions of the above described components can be processed by a processing entity (e.g., one or more processors) within the computer system in conjunction with execution of one or more other computer programs.

### The Kit

The invention also provides for a kit for using any of the various sensing devices or diagnostic platforms described herein.

The kit can be used to carry out any of the various methods as described herein.

In embodiments, the kit can comprise any one or more of the following: a sensing device, an electronic reading platform, a negative control, a positive control, sample buffer, a lancet (for use with fingerstick procedures), a nasal swab, a vial for collection of saliva, packing materials, and instructions for use. In embodiments, the sensing device can comprise a sensing cartridge with acousto-IgG™ or acousto-IgM™ sensor.

The instructions generally include one or more of: a description of the genetically engineered cells; methods for thawing or preparing cells; precautions; warnings; animal pharmacology; clinical studies; and/or references. The instructions can be printed directly on the container (when present), or as a label applied to the container, or as a separate sheet, pamphlet, card, or folder supplied in or with the container. Generally, a kit as described herein also includes packaging. In some embodiments, the kit includes a sterile container which contains a genetically engineered cells; such containers can be boxes, ampules, bottles, vials, tubes, bags, pouches, blister-packs, or other suitable container forms known in the art. Such containers can be made of plastic, glass, laminated paper, metal foil, or other materials suitable for holding cells or medicaments.

### EXAMPLES

The Examples set forth below are for illustrative purposes only and are not intended to limit, in any way, the scope of the present invention.

### Example 1

### Background

Severe acute respiratory syndrome coronavirus 2 (SARS-COV-2) leads to the infectious disease COVID-19, which was first reported in Wuhan, China in December 2019. The disease has since spread across the globe infecting over 200 countries. The lack of cheap, scalable, and rapid testing platform has contributed significantly to the spread of the diseases as countries struggle to identify patients and isolate them to prevent the wide spread of the disease before health care systems are overwhelmed. The problem is exacerbated by the presence of many asymptomatic infected patients. In the absence of proven antiviral drug therapies and vaccines, the current pandemic containment and mitigation strategy depends on isolation of the infected individuals and their close contacts in addition to social distancing through large scale lockdowns for the entire countries or communities. The latter has strained economies across the world, currently risks the availability of resources and has paralyzed the world in ways that will take years to recover from [5]. However, to mitigate the risk of having resurge in cases, increasing testing capacity and access is fundamental for the rapid identification and isolation of COVID-19 cases and containment of any new clusters.

The current technologies require skilled healthcare workers to operate, large, qualitative error-prone techniques that cannot be deployed in the field.

In this proposal, we aim to equip soldiers with a wearable (belt mounted) COVID-19 testing kit, one that tests for disease immunity. The proposed platform is a high throughput, high sensitivity point-of-care diagnostic platform for rapid detection of COVID-19 seroprevalence that covers necessary testing throughout the disease cycle.

### SARS-COV-2 Virology and Pathophysiology

SARS-COV-2 is an enveloped large positive-sense single-stranded RNA virus with genome of ~30 kb. It belongs to the genus betacoronavirus and has a diameter of 50-200 nm and spikes on its surface of length 20 nm that gives it a crown-like structure, a characteristic of coronaviruses (Zhou et al. 2020, Cascella et al., 2020). The SARS-COV-2 genome encodes for four major structural and function proteins: the spike(S), membrane (M), envelope (E) and nucleocapsid (N) proteins [6]. The S protein consists of 2 functional subunits, S1 and S2; S1 mediates the binding of the virus to the host cell receptor, while S2 contains other elements required for viral and cellular membrane fusion. The M protein is the most abundant structural protein that defines the shape of the virus. The N protein is the most abundantly shed viral protein during infection and can be detected in serum and urine samples within the first 2 weeks of infection. The smallest major structural protein, E protein, participates in viral assembly and pathogenesis [7]. COVID-19 patients show clinical manifestations in the form of fever, nonproductive cough, dyspnea, fatigue, and radiographic evidence of pneumonia. In addition, they show high production of leukocytes and elevated levels of cytokines, chemokines, and proinflammatory [8]. The immune response triggered against this virus involves the secretion of 3 types of immunoglobulins, IgG, IgM, and IgA, which are produced prior to a prolonged infection, and are essential to diagnose the presence of the virus within the body [9].

### Biomarkers

The SARS-COV-2 genome encodes for four major structural proteins: the spike(S), membrane (M), envelope (E) and nucleocapsid (N) proteins [6]. The S protein consists of 2 subunits, S1 and S2. S1 mediates the binding of the virus to the host cell receptor, while S2 contains other elements required for membrane fusion. The M protein is the most abundant structural protein that defines the shape of the virus. The N protein is the most abundantly shed viral protein during infection and can be detected in serum and urine samples within the first 2 weeks of infection. The smallest major structural protein, E protein, participates in viral assembly and pathogenesis [7].

Current RT-PCR assays detect the SARS-COV-2 RNA via envelope (E) and RNA-dependent RNA polymerase (RdRp) gene assays [10]. The assay is highly specific for SARS-COV-2 RNA and presents no cross-reactivity with other coronaviruses. In another approach, an RT-PCR assay was employed for the detection of RdRp/helicase (H) genes, which did not cross-react with other viruses [11]. Other RT-PCR assays were also developed that detect nucleocapsid protein (N) and ORF1b, which are highly conserved among other respiratory viruses. Thus, the assay could also bind to SARS-COV and other viruses [12].

Serological immunoassays have also been developed to detect antibodies for SARS-COV-2 in the serum or plasma of patients. These assays include rapid lateral flow immunoassay tests, ELISA, automated chemiluminescence immunoassay (CLIA) and others. These assays are mostly used to detect IgG and IgM antibodies. IgG is detectable starting 13 to 21 days after infection and persists for long durations. IgM response on the other hand occurs earlier, at around 10 days after infection, but then decreases rapidly after 35 days and disappears [13]. Data in FIG. 1 is collected from a literature survey showing the timeline of the disease with respect to the level of biomarkers (citations to the data points are in the appendix).

### Serological Testing

Serological or antibody-based testing can be complementary to real-time polymerase chain reaction (RT-PCR), which is the current gold standard for COVID-19 detection. These assays are good for determining seroprevalence and thus exposure in the community but unlike RT-PCR, are not suitable for diagnosis of patients in the acute phase of infection. First, antibodies are only released after the first week or infection. Second, current antibody-based rapid assays suffer in terms of their sensitivity and thus active cases can be easily missed. Misdiagnosis of active cases can have dire impacts on outbreak containment

The term “antibody” herein is used in the broadest sense and encompasses various antibody structures. Non-limiting examples of antibodies comprise monoclonal antibodies, polyclonal antibodies, multispecific antibodies (e.g., bispecific antibodies), humanized antibodies, fully human antibodies fully human antibodies as is understood in the art, and antibody fragments so long as they exhibit the desired antigen-binding activity. Antibodies can comprise comprises a monospecific antibody, a bispecific antibody, a trispecific antibody, or a multi-specific antibody. Non-limiting examples of antibody fragments comprise Fv, Fab, Fab′, Fab′-SH, F(ab′)2; diabodies; linear antibodies; single-chain antibody molecules (e.g. scFv); and multispecific antibodies formed from antibody fragments. In one embodiment, the invention comprise scFVs directed towards (and specific for) a target antigen or biomarker.

### Thickness Shear Mode (TSM) Biosensors

Quartz crystals are the most commonly used piezoelectric material for building transducers for gravimetry due to their desirable mechanical, thermal, chemical and electrical properties. AT-cut quartz produces bulk transverse shear waves with particle displacements parallel to the surface of the crystal and its electrodes [3, 15-17]. These AT-cut quartz oscillators are commonly termed thickness shear mode (TSM) resonators. When a small mass is deposited on the surface of a quartz crystal oscillator, the oscillator's resonance frequency decreases in direct proportion to the deposited mass as described by the classic Sauerbrey equation [4] for sensitivity of the resonator, which is provided below as Equation 1:

$$Δ {f}_{o} = \frac{2 {f}_{o}^{2}}{{( {ρ}_{Q} {μ}_{Q} )}^{1 / 2}} \frac{Δ m}{A}$$

Wherein fₒ is the fundamental resonant frequency of the quartz crystal, A is the surface area of the electrode on top of the crystal, μ_{Q} and ρ_{Q} are the shear modulus and the density of quartz [4]. While m is the mass deposited on sensor.

The novelty of mass sensing led to several thousand applications of TSM in biosensing, especially in point-of-care diagnostics. The specificity of the sensor relies on binding the molecular or protein of choice to the surface of the sensor for detection [18-21].

### High Sensitivity Molecular and Proteins Sensing μTSM Sensor

Our group has extensive experience using TSM sensors for biological sensing [2, 17, 18, 20, 22]. Recent work by our group has outlined the theoretical basis for increasing the sensitivity of TSM sensors validated by microfabricated prototypes used for biological experiments. Our work showed that high frequency quartz can be used to build miniaturized high sensitivity TSM sensors that operate at high Q-factors not damped by inharmonic modes. The loss of energy from inharmonic dampening was reduced by an optimal combination of sensor active area diameter, metal thickness and resonant frequency to create μTSM sensors with sensitivities that were orders of magnitude higher than commercially available TSM sensors [3, 18].

### Wearable, Miniaturized μTSM Systems

Advances in high efficiency and high frequency circuits for driving resonators has enabled a new generation of small and compact data acquisitions devices that can be also be Wi-Fi/Mobile phone connected, battery operated and light and small enough to be wearable. One embodiment of the present invention can utilize the openQCM™ system (Novaetech S.r.l., Pompei, Italy) a simultaneous frequency, dissipation and phase measurements platform based on AD8302 RF/IF Gain and Phase Detector™ (Analog Devices, Inc, Wilmington, Massachusetts). The platform provides fully integrated system for measuring gain/loss in the quartz sensor and phase difference between actuation and sensor response. In embodiments, the platform is designed to include a modular measurement cell allowing for testing a plurality of biomarkers on a single system, as shown in FIG. 2.

TABLE 1
Comparison of proposed Acousto-IgG ™ and
Acousto-IgM ™ with current approaches

| | Acousto-IgG ™/ | | | | | | | |
|---|---|---|---|---|---|---|---|---|
| | Acousto-IgM ™ | | Antigen | | Lateral Flow | | Enzyme linked Tests | |
| Target | IgG & IgM | | N protein | | IgG & IgM | | IgG, IgM and IgA | |
| | antibodies | | | | antibodies | | antibodies | |
| Sample | No user required | | Sampling | | Separate plasma or | | Separate plasma or | |
| preparation | sample prep. | | processing | | serum from specimen | | serum from specimen | |
| | | | for 1 minute | | by centrifugation | | by centrifugation or | |
| | | | Mix the sample | | Transfer whole | | sampling tubes with | |
| | | | gently by | | blood, serum or | | separation gel | |
| | | | pipetting | | plasma to the test | | sample processing and | |
| | | | Transfer the | | cassette for lateral | | coating with magnetic | |
| | | | sample to test | | flow capillary | | particles | |
| | | | cassette | | movement | | signal readout by a | |
| | | | Fluorescent | | Lateral flow visual | | plate reader | |
| | | | signal readout | | readout | | | |
| | | | preceded by | | | | | |
| | | | lateral flow | | | | | |
| Assay-to- | 5-10 | min | 15 | mins | 15-20 | min | 35 | min |
| result time | | | | | | | | |
| Size of | Wearable | | Large | | Small | | Large | |
| system | | | | | | | | |
| Sample-to- | 10 | min | ~20 | minutes | 30 | min | 1-5 | hrs |

| | | | | |
|---|---|---|---|---|
| result time | | | | |
| Result type | Quantitative | Qualitative | Qualitative | Quantitative |
| Sensitivity | 100% | 80% | 82-93.8% | 87.5-100% |
| Specificity | To be | 100% | 90.63-100% | 95-100% |
| | determined | | | |
| Cost (high, | Low | Low | low | High-Moderate |
| low) | | | | |

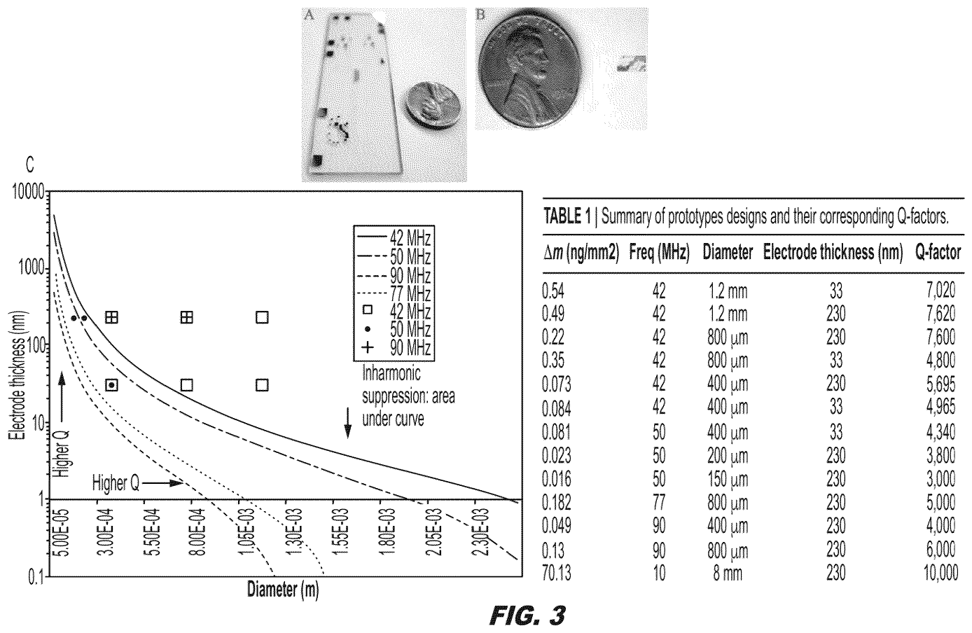

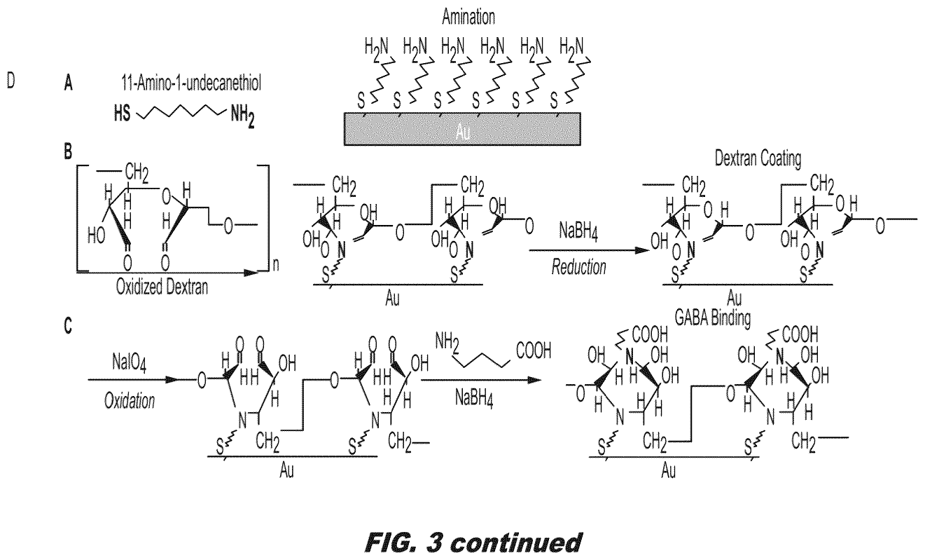

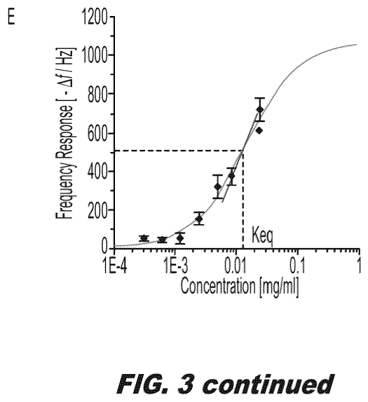

TSM piezoelectric sensors have shown great promise in bio-sensing applications but commercial TSM currently fall short in sensitivity and detection area as compared to competing sensing modalities such as surface plasmon resonance (SPR). We have investigated the design principles for improving sensitivity and lowering the detection area for TSM sensors operating in liquids for the purposes of monitoring cell adhesion in real time. The theoretical predictions were validated with fabricated prototypes operating in liquid and published [3, 18] as shown in FIG. 3A, B, C and table 1. Prior to that, we have developed TSM immunosensors to sense neurotransmitter gamma-aminobutyric acid (GABA) [2, 15]. Some of the key results are shown in FIG. 3D and E.

### Ink-Jet Processes for the Fabrication of Biosensors

Our group is engaged in developing inkjet processes for building microscale biosensors that are highly sensitive and cheap. Inkjet printers deposit small droplets of functional material, accurately and directly on a substrate. The resolution and capability of inkjet printing have greatly improved with recent advances in material dispensing technologies and electronic alignment. This method has several advantages over conventional photolithography techniques. This technique does not require masks or cumbersome clean-room equipment and requirements and has the ability to sequentially deposit multiple layers of different materials without perturbing the previously deposited layers. Accordingly, the deposition process is reduced to just few steps. To enable such fabrication, powerful machines capable of inkjet printing on a large-scale substrate can be used. One example of such a machine includes the Fluid FM® BOT (Cytosurge AG, Glattbrugg, Switzerland), a system capable of nanoinjection with a vast variety of materials of choice selectively into any substrate at nanoscale resolution. An alternate system is the Dimatix® Materials Printer (Fujifilm, Minato City, Tokyo, Japan) which is capable of scaling designs to 8×11 inch areas while maintaining a micron resolution printing with 8 materials printed simultaneously [23, 24]. Preliminary results from this inkjet printing approach are shown in FIG. 4. Without being bound by theory, such inkjet techniques can pattern electrodes of desired thickness and dimensions on the quartz resonators and achieve the desired sensitivities.

Without being bound by theory, disclosed herein is a low cost, high sensitivity wearable diagnostic platform for rapid detection of seroprevalence of COVID-19 that covers necessary testing throughout the disease cycle (FIG. 5). Embodiments of the presently disclosed platform perform serological blood test via fingerstick using a belt mounted system. The sensor cartridge comprises a thickness shear mode (TSM) transducer where mass sensing can be used for detection. In embodiments, a sandwich assay is utilized that comprises immobilized S protein on the surface and ant-IgM and IgG in buffer topping the sensor to detect IgM and IgG separately (as illustrated in FIG. 5-2a&b).

Aim #1: Without being bound by theory, the presently disclosed serological blood test assay permits low cost, high throughput, high sensitivity point-of-care diagnostic platform for rapid detection of antibodies to COVID-19. In embodiments, the diagnostic platform can be used to assess seroprevalence in the community and recovery stage in COVID-19 patients.

Without wishing to be bound by theory, TSM sensors can utilize a sandwich assay (S protein immobilized on the sensor surface loaded with IgG/IgM and IgG/IgM antibodies using commercially available 10 MHz quartz crystals. Without being bound by theory, a high sensitivity antibody COVID-19 sensor can be incorporated into a millimeter scale TSM biosensor. In embodiments, assays can comprise at two antibodies for SARS-Cov-2 as a hand-held, point-of-care diagnostic.

Impact: Without being bound by theory, embodiments include a quantitative bioelectronic antibody assay for COVID19 that can comprise a hand-held sensor usable by military personnel deployed in a mission.

Aim #2: Without being bound by theory, the Acousto-IgG™ and Acousto-IgM™ platform will be highly specific and stable.

Aim #2A: The cross reactivity of the platform to human IgG and IgM can be tested. The aim targets testing the serologic cross reactivity of available anti-IgG and anti-IgM antibodies.

Innovation: In embodiments, high specificity anti-IgG and anti-IgM ensures high accuracy of the platform.

Challenge: Cross reactivity can significantly impact the sensitivity of platform especially when the sensing is in blood.

Impact: Without being bound by theory, the optimal choice of anti-IgG and anti-IgM enables accurate estimation of disease progression.

Aim #2B: Without wishing to be bound by theory, the shelf life of the sensing cartridge in room can be stabilized via stabilizing the surface bound S protein and IgG/IgM antibodies.

Innovation: Without wishing to be bound by theory, several stabilizing solutions for the capture surface device will extend the shelf life at room temperature. In embodiments, such stabilizers include, Sucrose, LB Medium and Blocking buffer.

Deploying the IVD platform in the field requires accounting for the conditions that can affect the stability of the antibodies.

Impact: Without being bound by theory, this disclosure provides for a long shelf life of the sensor cartridges in Acousto-IgG™ and Acousto-IgM™ platform.

Aim 3—Without being bound by theory, a functional product can be developed using appropriate ISO design map, regulatory map, software architecture map, risk management studies, manufacturing processes etc.

Without being bound by theory, the sensor hardware and accompanying software can comprise a wearable, point-of-care in vitro diagnostic platform.

### Strategy

Aim #1: Develop and test the sensitivity of TSM sensors to sandwich assay (S protein immobilized on the sensor surface loaded with IgG/IgM and IgG/IgM antibodies using commercially available 10 MHz quartz crystals.

Experimental Design: Without being bound by theory, we can utilize immobilization methodology to produce stable anchoring of bioactive antigen molecules with reproducible orientations to ensure consistency in coating. Briefly, amination of gold surface via self-assembly mono-layer (SAM) of 11-amino-1-undecanethiol. This is followed by covalent attachment of streptavidin and then biotinylated S protein are attached via streptavidin-biotin bond. Each stage can be monitored via TSM sensor response, atomic force microscopy (AFM) and X-ray photoelectron spectroscopy (XPS) [25]. The sensor can be topped with 1 mg/ml anti-IgG or anti-IgM (depending on what sensor cartridge we are testing). We can test various concentrations IgG/IgM (1×10−4-1 mg/ml) and build a frequency/concentrations curve to help predict antibody concentration FIG. 4E [2]. Data Acquisition: We can openQCM devices and software during this phase [3, 18].

Sensitivity to anti-bodies: Effectively, the sensor can measure the binding of anti-IgG/IgM to the S protein bound IgM/IgG. Although the concentration of IgM and IgG vary widely among different patients, the median has been reported as reaching peaks at 16-20 days after illness onset at 7.25 μg/mL for IgM, while median concentration of IgG peaked during 21-25 days after illness onset at 16.47 μg/mL. Our data show the linear regime of the sensor is centered around 10 μg/ml as shown in the dose-response curve of FIG. 3. For a 10-MHz AT-cut quartz crystal at room temperature, sensitivity is approximately equal to 4.4 ng/(cm2·Hz). Crystals are typically 10 mm in diameter resulting in a sensitivity of 3.45 ng/Hz. A drop of blood from a pin prick is estimated to be 50 μL which would contain 362 ng & 882 ng of IgM and IgG, respectively. Also, without being bound by theory, our platform can reach frequency of 60 MHz while reducing the sensing area of TSM to 30 μm which can achieve 7 orders of magnitude increase in sensitivity [3, 18]. The latter ensures a high sensitivity platform with a small sample of blood (obtained by finger prick).

Prophetic Results and Interpretation: Without being bound by theory, we can determine a calibration curve for Acousto-IgG™ and Acousto-IgG™ using different concentrations of IgG and IgM antibodies in standard solutions. Under experimental conditions, this calibration curve can be used to determine the concentrations of IgG and IgM in the blood sample. Replicates of 5 each for 10 distinct concentrations of IgG and IgM, at an α=0.05 results in statistical power of 0.89.

TABLE 2
Desirable specifications for imprecision, bias and total error

| Analyte | CV_{W} | CV_{G} | I (%) | B (%) | TE (%) |
|---|---|---|---|---|---|
| IgG | 4.5 | 16.5 | 2.3 | 4.3 | 8.0 |
| IgM | 5.9 | 47.3 | 3.0 | 11.9 | 16.8 |

The desirable specifications for imprecision, bias and total error are listed in Table 2 (from Biological variations references database https://www.westgard.com/biodatabase2.htm). Imprecision or analytical variation should be less than half of the within-patient coefficient of variation (CVW). I<0.5 CVw. This same specification has to be maintained for screening, diagnosis and case finding when a fixed cut-off point is used to define a pathological or healthy condition [EK Harris, Am J Clin Path, 1979]. Analytical bias can be maintained within one quarter of within-plus between-subject components. We can determine the (+) or (−) for the presence of IgM or IgG from the lowest concentration recorded frequency/concentrations curve (Table 3).

TABLE 3
The result of the patient test using the disclosed
platform can fit with the table to better assess the
clinical significance of the test and state of the patient.

| IgM | IgG | Clinical Significance |
|---|---|---|
| − | − | Patient potentially is in the |
| | | early window of infection |
| + | − | Patient may be in intermediate |
| | | stage of infection |
| + | + | Patient in late phase of |
| | | infection |
| − | + | Patient may be in late stage or |
| | | early stage of recurring |
| | | infection |
| + | − | Patient may be in intermediate |
| | | stage of infection |
| − | + | Patient is in the convalescent |
| | | stage (recovered) |
| + | + | Patient may be in recovery |
| | | stage of infection |

Alternative Approaches: Without being bound by theory, the calculated sensitivity of the commercial crystals can be capable of sensing IgM and IgM from COVID-19 positive patients as indicated by our estimates in the previous section. In the event, we need to enhance the sensitivity due to variations between patients, we can use the techniques outlined in our most recent publication [3] build our own sensors with enhanced sensitivity (see background and significance section). We can obtain blank quartz crystals (such as those from Xeco Inc., Cedar city, UT, USA). Inkjet printing can be used to pattern electrodes on surface of the crystal (see preliminary data for layer and electrode control). The same technology can pattern an interface board. The printing process can be done via a commercially available material printer (e.g., DMP-2850, Fujifilm Dimatix, USA) fitted with a piezo-driven 16-nozzle print-head. The conductive patterns and coatings can be printed using a 1 μL cartridge, while the passivation layers can be printed using a 10 μL cartridge as shown in FIG. 4.

Aim #2A: Test the cross reactivity of the platform to human IgG and IgM. The aim targets testing the serologic cross reactivity of commercially available anti-IgG and anti-IgM antibodies. More rigorous cross-reactivity assessments can be performed under aim #3.

Experimental Design: Surfaces can be coated as described in aim #1. Without being bound by theory, cell-free blood samples can be used to test the specificity of the Acousto-IgG™ and Acousto-IgM™ diagnostic platforms. Human serum samples from healthy individuals can be spiked with low, mid and high concentrations of human IgG and human IgM (concentration range 1×10−4-1 mg/ml). We can then test for the impact of endogenous interferences such hemoglobin, biotin, bilirubin, human anti-mouse antibody (HAMA), intralipid and rheumatoid factor, human IgG (for the samples with IgM), human IgM (for serum samples with IgG) and human IgA. Each sample can be tested in duplicate. More detailed interference assessments with exogenous molecules and assessments of hook effect can be also be performed.

Membrane for isolating cells from blood: We can incorporate an electrospun filter that efficiently removes blood cells from whole blood. The design is based on poly(ethylene terephthalate) (PET)/poly(vinyl pyrrolidone) (PVP) blend. Previous work has shown 80%/20% blend of PET/PVP respectively produces a mean fiber diameter of 0.92 μm with a mean pore size of 4.67 μm, which is sufficient to retain WBCs and RBCs [26]. The additional decrease in pore size could retain platelets as well.

Prophetic Results and Interpretation: Interference can be considered significant if response of the sensor in the presence of the interfering agent exceeds ±10% of initial value. We will then determine the highest concentration of the interference that showed a non-significant effect.

Aim #2B: Without being bound by theory, the shelf life of the sensing cartridge in room can be stabilized via stabilizing the S protein and anti-IgG and IgM.

Experimental Design: The process for adding the stabilizers comprises covering the prepared sensor surface with 1) blocking buffer (Thermo Fisher Scientific, USA) 0.15 M. 2) (5% w/v) sucrose (Acros). 3) coating stabilizer and blocking buffer diluted 1:1 with water. The devices can then be aspirated and dried. All devices can be stored at 50° C. to age the coating (1 day at 50° C. is equal to about 6.5 days at room temperature). We can assess the stability of the coating at 10 days, 3 weeks, 3 months & 6 months [27]. Without being bound by theory, embodiments can comprise an 18-month shelf life. Besides the in-use stability assessment of reagents in the above experiments, we can also assess the stability of the reagents under the following conditions:

Shelf-life stability-Reagent shelf life can be assessed by real-time stability testing, with reagents stored at the specified storage temperature.

Stress testing—the reagents can be cycled through a temperature of 4° C. and ambient temperature to mimic shipping conditions. A separate group of reagents can be cycled through light conditions to mimic shipping conditions. They can then be placed under normal storage conditions and their in-use stability assessed.

Prophetic Results and Interpretation: Without being bound by theory, the samples covered by sucrose have the highest stability.

Aim 3—Without being bound by theory, a functional product can be developed using appropriate ISO design map, regulatory map, software architecture map, risk management studies, manufacturing processes etc.

In embodiments, the biosensor product can comprise a wearable form factor under 100-150 g. The biosensor can be configured for military use conditions with disposable serology cartridges under variable weather conditions not limited to temperature, humidity, and atmospheric pressure.

Aim 4—Testing and validation of the Acousto-IgG™ and Acousto-IgM™ diagnostic systems towards.

We can use the Serology template for manufacturers provided by the FDA (guidance document issued May 2020-Policy for Coronavirus Disease-2019 Tests During the Public Health Emergency (Revised)—Immediately in Effect Guidance for Clinical Laboratories, Commercial Manufacturers, and Food and Drug Administration Staff) for validation of the acousto-IgG™ and acousto-IgM™ diagnostic systems. Validation can be performed using blood samples obtained from 2 different sites—the CLIA-waived laboratory at the Biodesign Institute in ASU, Tempe, AZ and from Dr. Zaraket lab in AUB, Beirut, Lebanon. Control solutions that will accompany the diagnostic systems include solutions with low & high IgG (positive controls) and no IgG (negative control), low & high IgM (positive control) and no IgM (negative control).

| Kit components (example) | Manufacturer |
|---|---|
| Test cartridge with acousto- | Open-QCM and Grace |
| IgG ™ or acousto-IgM ™ | Microsystems LLC |
| sensor | |
| acoust-IgG ™ or acousto- | Open-QCM |
| IgM ™ reader platform | |
| Negative control | Agrisera AB, Sweden |
| Positive control | Agrisera AB, Sweden |
| Sample buffer (bottle) | Grace Microsystems |
| | LLC |
| Lancet (for fingerstick only) | Grace Microsystems |
| | LLC |
| Instructions for Use leaflet | Grace Microsystems |
| | LLC |
| Packing materials | Grace Microsystems |
| | LLC |

Cross-reactivity—The FDA recommends testing for cross-reactivity against the following antibodies-anti-influenza A (IgG and IgM), anti-influenza B (IgG and IgM), anti-HCV (IgG and IgM), anti-HBV (IgG and IgM), anti-Haemophilus influenzae (IgG and IgM), anti-229E (alpha coronavirus), anti-NL63 (alpha coronavirus), anti-OC43 (beta coronavirus), anti-HKU1 (beta coronavirus), ANA, anti-respiratory syncytial virus (IgG and IgM) and anti-HIV. We can test a minimum of 5 individual samples for each disease/infectious agent/antibody class listed above (as per FDA recommendation). We can prepare spiked samples with the IgM or IgG antibodies for the underlying conditions. We can use commercially available IgM or IgG antibodies for the underlying conditions panels collected prior to the COVID-19 pandemic to ensure the panels are SARS-COV-2 antibody negative.

Power analysis—For n=5 trials, and a of 0.05, 10% variability and a difference in means of 20%, the statistical power of the above comparison was estimated to be 0.89.

If a significant false positive rate (>5%) is observed, we can test different anti-human-IgG and anti-human-IgM identify the candidate anti-human-IgG and anti-human-IgM that minimizes the cross-reactivity.

Class-specificity testing-Since our Acousto-IgG™ or Acousto-IgM™ will quantitatively assess the different classes of immunoglobulins, we can perform the following class-specificity testing as recommended by the FDA. We can use the dithiothrietol (DTT) assay on both of the above diagnostic systems to assess their class-specificity, where the signal due to IgM either decreases or becomes negative upon application of DTT while the signal due to IgG remains unaffected. We can test both Acousto-IgG™ and Acousto-IgM™ with 5 samples (IgG/IgM, +/+) and 2 replicates each (as recommended by the FDA template). Without being bound by theory, we will see 100% agreement between the results of the diagnostic systems and the expected result of DTT treatment (−/+) to IgM/IgG, +/+. To confirm DTT activity, a positive control test can also be included.

Clinical agreement study-We can test prospectively collected SARS-COV-2 antibody positive specimens from patients that have been previously confirmed infected by SARS-CoV-2 RT PCR. These specimens can be purchased either from the CLIA-waived laboratory in the Biodesign Institute at ASU or directly from the CDC. The specimens can be accompanied by basic information such as the population from which the sample was drawn and the comparator method, specimen collection date, date of onset of symptoms (if present/known), and comparator method to confirm patients as SARS-COV-2 infected or not infected.

Patient samples eligible for this study ca be from patients who are 18-99 years old, of all sexes including healthy volunteers.

### Criteria for Inclusion and Exclusion

### Inclusion Criteria:

Individuals who have experienced symptoms of COVID-19 and have been tested using a CDC approved or FDA registered and listed nucleic acid based test within 1 year of Feb. 1, 2020.
Individuals who are at the time of enrollment in the study currently or in the recent past (3 weeks) exhibiting symptoms of COVID-19.
Individuals capable of performing a finger stick blood drop draw and placing it on the sample well.
Individuals that have interacted with a COVID-19 positive individual and are still exhibiting symptoms will be tested by the Arizona Department of Health Services with a CDC approved or FDA registered nucleic acid-based device.

### Exclusion Criteria:

Individuals incapable of pricking their finger and placing a drop of blood into a sample well.
Pregnant woman are not excluded if they meet the inclusion criteria and age requirements.

We can collect a minimum of 30 IgG positive and 30 IgM positive samples and 30 IgG negative and 30 IgM negative samples as deemed acceptable by the FDA. The samples can be generated, collected and sourced from the CLIA-waived laboratory in the Biodesign Institute at ASU and will be collected prospectively after an IRB approval from ASU, Tempe, AZ.

In addition, we can also place an Acousto-IgG™ and Acousto-IgM™ system in the above laboratory. We can have the patients perform a blood draw using a lancet and use the above diagnostic systems. Both the patients and the laboratory personnel can be blinded to the results from the above diagnostic systems. The results of the blood samples collected by the lab personnel can be independently analyzed from the blood samples drawn by the patients using a lancet. As recommended by the serology template for manufacturers from FDA, we can use the following tables to quantitatively assess the positive percent agreement (PPA) and negative percent agreement (NPA):

| | Comparator method/Clinical truth | | |
|---|---|---|---|
| | | Positive | Negative |
| Acousto- | Positive | A | B |
| IgG ™ | Negative | C | D |

| | Comparator method/Clinical truth | | |
|---|---|---|---|
| | | Positive | Negative |
| Acousto- | Positive | A | B |
| IgM ™ | Negative | C | D |

The PPA for each of the above diagnostics is determined by A/(A+C) and the NPA for each of the above diagnostics is determined by D/(D+B). Independent analysis of NPA and PPA from the blood samples collected by the lab personnel and corresponding blood samples obtained by fingerstick from patients using the lancet will allow us to validate the point-of-care claim.

Matrix equivalency-We can test 4 different sample matrices-(a) fingerstick cell-free, whole blood (b) EDTA plasma (c) anti-coagulant 1 and (d) anti-coagulant 2. Each of the sample(s) can come from the same donor from the CLIA-waived laboratory in the Biodesign institute at ASU. As required by the FDA template for serology manufacturers, samples that were assessed negative with the above 2 Acousto-IgG™ and Acousto-IgM™ diagnostics can subsequently be spiked with low SARS-Cov-2 IgG & IgM antibodies (separate samples) and moderate SARS-Cov-2 IgG & IgG (separate samples). Negative samples for each of the 4 specimen matrices can be spiked with the same amount of SARS-Cov-2 IgG and IgM (separate samples) to allow for comparison. We can test 2 replicates of 5 samples under each condition (concentration of antibodies)—negative, low and moderate. Therefore, we will have 30 samples to be tested under each matrix/specimen condition. We can repeat this for each of the 4 specimen conditions each for IgG and IgM respectively.

Specimen matrix 1 (IgG)

| | | | Low | Moderate |
|---|---|---|---|---|
| Sample | replicate | Negative | postive | positive |
| Sample 1 | 1 | | | |
| | 2 | | | |
| Sample 2 | 1 | | | |
| . . . | 2 | | | |
| Sample 5 | 1 | | | |
| | 2 | | | |

Specimen matrix 1 for (IgM)

| | | | Low | Moderate |
|---|---|---|---|---|
| Sample | replicate | Negative | postive | positive |
| Sample 1 | 1 | | | |
| | 2 | | | |
| Sample 2 | 1 | | | |
| . . . | 2 | | | |
| Sample 5 | 1 | | | |
| | 2 | | | |

Aim 5—Optimize the hardware and accompanying software to a wearable, point-of-care in vitro diagnostic platform.

In various embodiments, sensing cartridge of the presently disclosed platform comprise molecular moieties on the sensor, given dimensions of the gold electrode (thickness of the gold film and diameter of the gold electrode), given acoustic parameters of the sensor (resonant frequency, Q-factor etc), or a combination thereof. The hardware (the electronic reading platform) and the software interface can be further configured to comprise a wearable in vitro diagnostic.

In embodiments, the electronic reading platform interfaces with the sensing cartridge through an USB interface and with an external mobile phone through a wireless Bluetooth interface. The electronic reading platform can be optimized for any one or more of the following features:
Form factor that will allow the attachment of the electronic reading platform to be attached to a belt in the waist while allowing for free movement of the wearer. In some embodiments, the wearer can comprise a soldier. It can also be optimized for ease of hand-held operation and improved robustness against damages during physical handling, out in the open field.
Reduce the weight of the device to a few tens of grams excluding battery.
Enhance battery-life such as through a combination of software and hardware enhancements that minimizes power consumption during use and allows the device to operate in a low-power sleep mode when not in use.
Eliminate temperature rise due to electronic heating when it is used to analyze a large batch of samples that could potentially corrupt the sensor performance.
Enhance the user experience through improved graphical user interface (GUI) and improve reliability of wireless communication between the hand-held electronic reader and the mobile phone; and
In a limited number of patients (50 positive samples and 50 negative samples each for IgG and IgM), we will run validation experiments with similar inclusion and exclusion criteria used in the “clinical agreement study” detailed in aim #3 to verify the point-of-care claim for both the Acousto-IgG™ and Acousto-IgM™ diagnostic systems subsequent to all the above enhancements.

### REFERENCES CITED IN THIS EXAMPLE

1. Lauer, S. A., et al., The Incubation Period of Coronavirus Disease 2019 (COVID-19) From Publicly Reported Confirmed Cases: Estimation and Application. Ann Intern Med, 2020.
2. Wang, T. and J. Muthuswamy, Immunosensor for detection of inhibitory neurotransmitter gammaaminobutyric acid using quartz crystal microbalance. Anal Chem, 2008. 80 (22): p. 8576-82.
3. Khraiche, M. L., J. Rogul, and J. Muthuswamy, Design and Development of Microscale Thickness Shear Mode (TSM) Resonators for Sensing Neuronal Adhesion. Front Neurosci, 2019. 13: p. 518.
4. Sauerbrey, G., The use of quartz crystal oscillators for weighing thin layers and for microweighing applications. Z. Phys, 1959. 155: p. 206-222.
5. Li, R., et al., Substantial undocumented infection facilitates the rapid dissemination of novel coronavirus (SARS-COV2). Science, 2020.
6. Li, X., et al., Molecular immune pathogenesis and diagnosis of COVID-19. J Pharm Anal, 2020.
7. Vashist, S. K., In Vitro Diagnostic Assays for COVID-19: Recent Advances and Emerging Trends. Diagnostics, 2020. 10 (4): p. 202.
8. Rothan, H. A. and S. N. Byrareddy, The epidemiology and pathogenesis of coronavirus disease (COVID-19) outbreak. J Autoimmun, 2020. 109: p. 102433.
9. Jacofsky, D., E. M. Jacofsky, and M. Jacofsky, Understanding Antibody Testing for COVID-19. J Arthroplasty, 2020.
10. Corman, V. M., et al., Detection of 2019 novel coronavirus (2019-nCOV) by real-time RT-PCR. Euro Surveill, 2020. 25 (3).
11. Chan, J. F., et al., Improved molecular diagnosis of COVID-19 by the novel, highly sensitive and specific COVID-19-RdRp/Hel real-time reverse transcription-polymerase chain reaction assay validated in vitro and with clinical specimens. J Clin Microbiol, 2020.
12. Chu, D. K. W., et al., Molecular Diagnosis of a Novel Coronavirus (2019-nCOV) Causing an Outbreak of Pneumonia. Clin Chem, 2020. 66 (4): p. 549-555.
13. Tan, W., et al., Viral Kinetics and Antibody Responses in Patients with COVID-19. medRxiv, 2020: p. 2020.03.24.20042382.
14. To, K. K., et al., Temporal profiles of viral load in posterior oropharyngeal saliva samples and serum antibody responses during infection by SARS-COV-2: an observational cohort study. Lancet Infect Dis, 2020.
15. Ergezen, E., et al., Real time monitoring of the effects of Heparan Sulfate Proteoglycan (HSPG) and surface charge on the cell adhesion process using thickness shear mode (TSM) sensor. Biosensors and Bioelectronics, 2007. 22 (9-10): p. 2256-2260.
16. Lu, F., et al., Finite element analysis of interference for the laterally coupled quartz crystal microbalances. Sensors and Actuators a-Physical, 2005. 119 (1): p. 90-99.
17. Wang, T. T., et al., Immobilization and characterization of gamma-aminobutyric acid on gold surface. Journal of Biomedical Materials Research Part A, 2006. 79A (1): p. 201-209.
18. Khraiche, M. and J. Muthuswamy, Multi-modal biochip for simultaneous, real-time measurement of adhesion and electrical activity of neurons in culture. Lab Chip, 2012. 12 (16): p. 2930-41.
19. Khraiche, M. L., N. Jackson, and J. Muthuswamy, Early onset of electrical activity in developing neurons cultured on carbon nanotube immobilized microelectrodes. Conf Proc IEEE Eng Med Biol Soc, 2009. 2009: p. 777-80.
20. Khraiche, M. L., A. Zhou, and J. Muthuswamy, Acoustic sensor for monitoring adhesion of Neuro-2A cells in real-time. J Neurosci Methods, 2005. 144 (1): p. 1-10.
21. M L Khraiche, A Zhou, and J Muthuswamy, Acoustic sensor for monitoring adhesion of Neuro-2A cells in real-time Journal of neuroscience methods, 2004. 144 (1): p. 1.
22. Zhou, A. and J. Muthuswamy, Acoustic biosensor for monitoring antibody immobilization and neurotransmitter GABA in real-time. Sensors and Actuators B: Chemical, 2004. 101 (1): p. 8-19.
23. Qiu, S. and C. Zhou, Organic Printable Electronic Materials. Printed Electronics, 2016: p. 21-53.
24. Su, W., Encapsulation Technology for Organic Electronic Devices. Printed Electronics, 2016: p. 287-315.
25. Wang, T., et al., Immobilization and characterization of gamma-aminobutyric acid on gold surface. J Biomed Mater Res A, 2006. 79 (1): p. 201-9.
26. Shahrabi, S. S., J. Barzin, and P. Shokrollahi, Blood cell separation by novel PET/PVP blend electrospun membranes. Polymer Testing, 2018. 66: p. 94-104.
27. Standard Guide for Accelerated Aging of Sterile Medical Device Packages. ASTM International Designation. F 1980-02.

### Example 2

Brief Summary: The world continues to struggle to contain the spread of COVID19, first reported in Wuhan, China in December 2019. This global public health crisis risks overwhelming healthcare systems everywhere with a highly contagious acute respiratory syndrome that has to date claimed the lives of hundreds of thousands of people. The lack of cheap, scalable, and rapid testing platform has contributed significantly to the continued lockdown measures as we struggle to identify patients that have acquired an immunity to the disease to quantitively assess the state of herd immunity. This disease also has huge national security implications. The lack of readily available test kits and the need for specialized laboratory equipment for testing can have a significant impact on the military along the entire chain of command. Therefore, there is a need for a point-of-care in vitro diagnostic system that is wearable (or easily deployable in remote areas), readily available and can be used by untrained personnel. It becomes important to have tests that can identify the prevalence of the disease at every stage of progression. Therefore, CDC in partnership with state, local and territorial health agencies, public and private healthcare providers routinely conducts seroprevalence studies to track the status of disease in the population.

In various embodiments, the invention reported here includes a hand-held/wearable, high accuracy and easy to use diagnostic platform to enable the assessment of serological prevalence of COVID19. The current testing technologies are often qualitative, require healthcare workers to operate, prone to error and too large to be deployed in the field. In embodiments, the disclosed diagnostic platform comprises two distinct variants, Acousto-IgG™ and Acousto-IgM™ for the detection of IgG and IgM antibodies to SARS-COV-2 and requires no medical training to operate, performs the test in minutes using a fingerstick and transmits data directly to Wi-Fi enabled devices. The core technology involves the use of acoustic waves to achieve sensitivities that are orders of magnitude higher than the current techniques such as enzyme linked immunosorbent assay (ELISA), chemi-luminescent immunoassay (CLIA) or lateral flow assays that are often semi-quantitative. Other advantages over current technologies include a rapid, quantitative read-out (within minutes), as opposed to the qualitative read-out that take 15-30 min with current techniques. The hand-held, point-of-care nature of this invention will mitigate the need for samples to be transported over long distances to specialized laboratories or for patients to make risky trips to local diagnostic laboratories.

Introduction: The invention builds on the successful experience of the inventors in developing highly sensitive, label-free, acoustic sensors for monitoring cellular adhesion, neurotransmitter concentrations and other biomolecular events. We have successfully developed highly sensitive acoustic sensors for the detection of anti-bodies to GABA (gamma-aminobutyric acid, a widely prevalent neurotransmitter in the brain). The invention reported here pertains to the fields of—in vitro diagnostics, acoustic sensors, biomolecular detection, millimeter and microscale electromechanical systems. Most recently, the inventors developed a technique (M. Khraiche, J. Rogul, and J. Muthuswamy, Design and Development of Microscale Thickness Shear Mode (TSM) Resonators for Sensing Neuronal Adhesion, Front. Neurosci., 4 Jun. 2019) to enhance the sensitivity of the acoustic sensors using physical principles of bulk wave resonators leading to optimal design of electrode dimensions and thickness of the quartz resonators for achieving desired sensitivity. The invention reported here builds on all of this work.

In embodiments, the disclosed invention comprises a wearable, in vitro diagnostic platform for detecting SARS-COV-2 antibodies. Without wishing to be bound by theory, the disclosed diagnostic platforms and disposable sensor cartridges will equip soldiers with a much needed point-of-care technology to identify immune individuals and serological prevalence in a population regardless of proximity or access to medical centers.

### SUMMARY OF THE INVENTION

Serological or antibody-based testing for SARS-COV-2 (Severe Acute Respiratory Stress-Coronavirus-2) is critical to assess seroprevalence and also track efficacy in vaccine development. Here, we disclose a point-of-care in vitro diagnostic (IVD) technology that will address current challenges in access/availability, deployment and communication of results, ease of use, scalability to large and remote populations.

OPPORTUNITY: Public and private health services, contract research organizations (CROs), and pharmaceutical companies need regular data on seroprevalence to track disease progression and also assess the efficacy of new vaccines and therapeutics currently under development. Current diagnostic technologies require skilled workforce to operate and interpret results, often 24-48 hr turnaround times, and hard to deploy in remote locations. Patients are often reluctant to visit diagnostic laboratories and health care providers during this pandemic significantly increasing the time, cost and failure rates of such seroprevalence studies.

SOLUTION: We disclose here a hand-held or wearable, rapid (<1 min), low-cost IVD. In embodiments, the IVD detects IgG and IgM antibodies for the SARS-Cov-2 virus, with high sensitivity and specificity that can be rapidly deployed and operated without the requirement of skilled laboratory personnel. The proposed technology can also communicate results back to health care providers through mobile phone or other hand-held computing platforms. This IVD technology can enable virtual clinical trials of new vaccines and therapeutics. In embodiments, the IVD platform can also be adapted at a later stage to track other biomarkers.

The Assure system is a qualitative assessment using lateral flow assays. The presently disclosed IVD will provide rapid, quantitative assessment of both IgG & IgM.

In various embodiments, the present invention comprises one or more of the following:
Millimeter or microscale electrodes on bulk acoustic wave resonators allow for tuning of sensitivity of conventional acoustic sensors to what is optimal to detect IgG and IgM antibodies in blood samples.
The chemistry on surface of the electrodes of the resonators determines the selectivity of the sensor to the antibodies to SARS-Cov-2.

In embodiments, the presently disclosed technology provides for any one or more of the following advantages:
Significantly enhanced sensitivity to antibody detection
Eliminate transporting blood samples.
Reduce time from sample collection to result from several hours to <10 min.
Reduce time and cost for clinical trials by enabling virtual clinical trials.
Quantitative assessment that will allow advanced informatic tools for predictive modeling.
As easy to use as a glucometer.
Eliminates the need for trained personnel and patient visits to labs.

In various embodiments the presently disclosed invention provides a quantitative assessment of both IgG and IgM antibodies rapidly within seconds. In addition, compared to most of the competition, embodiments require no trained personnel (will be as easy to use as a glucometer).

### Example 3

### Non-Limiting, Exemplary Data for the Acoustic Resonator-Based Sensor to Detect Antibody to Spike RBD Protein of SARS-COV-2 Virus

### Summary: Effect of Alkanethiol Composition on QCM Response to Covid-19 Spike RBD Antibody

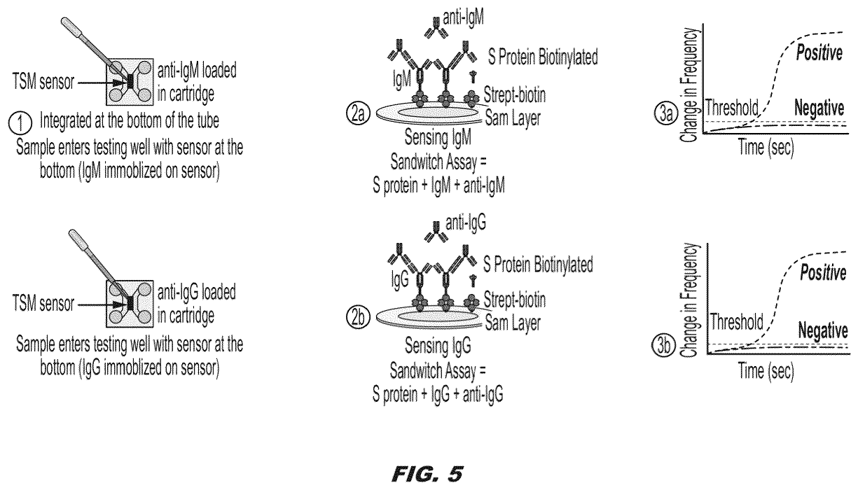

FIGS. 7-9 show immobilization chemistry to sense antibodies to Spike-protein of the SARS-COV-2 virus

QCM sensor chemistry can be tailored to desired dynamic range.

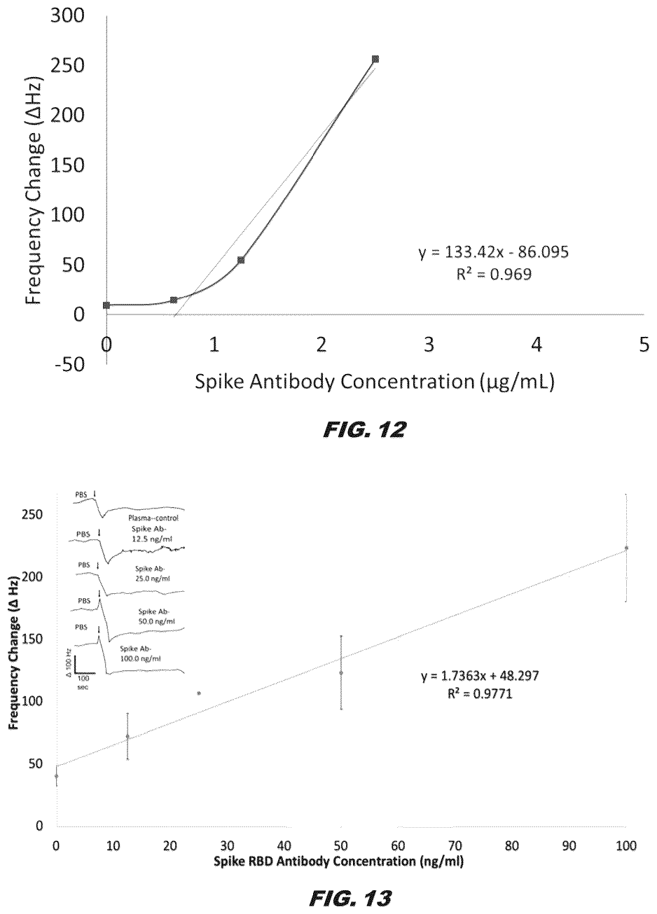

FIGS. 12-13 indicate QCM sensor was responsive to [Spike Ab] in plasma when diluted in PBS.

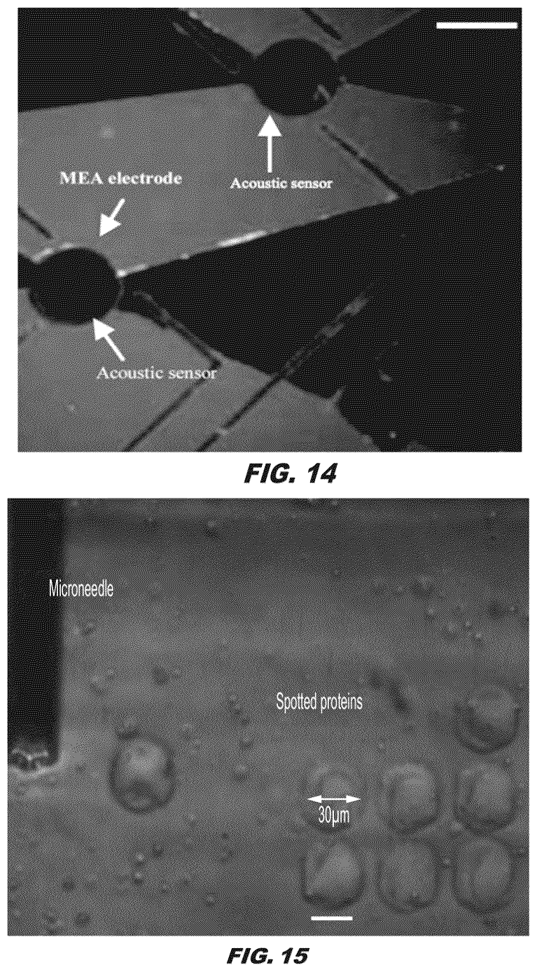

FIGS. 14-15 show multi-channel micro-resonators and spotting process

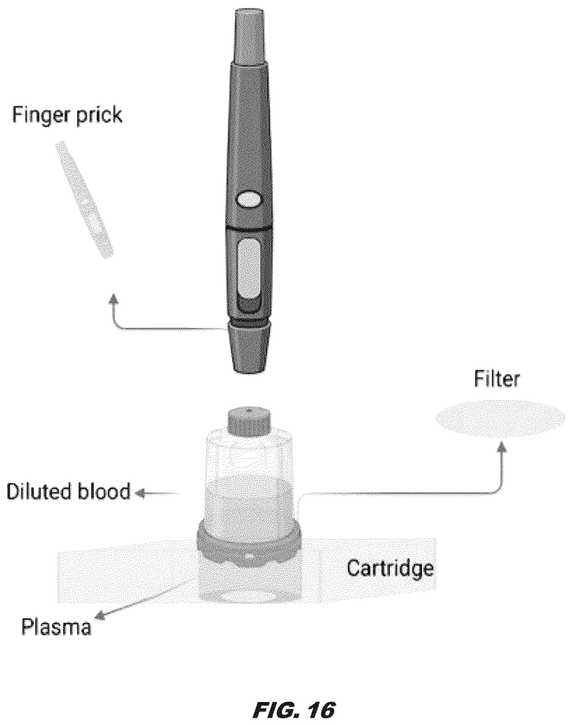

FIG. 16 shows scheme for blood draw using lancet and filtration before sensing

### Chemicals Used:

EDC-1-ethyl-3-(-3-dimethylaminopropyl) carbodiimide
NHS—N-Hydroxysuccinimide
11-Mercaptoundecanoic acid (MUA)
9-Mercapto-1-nonanol
PBS-phosphate buffered saline

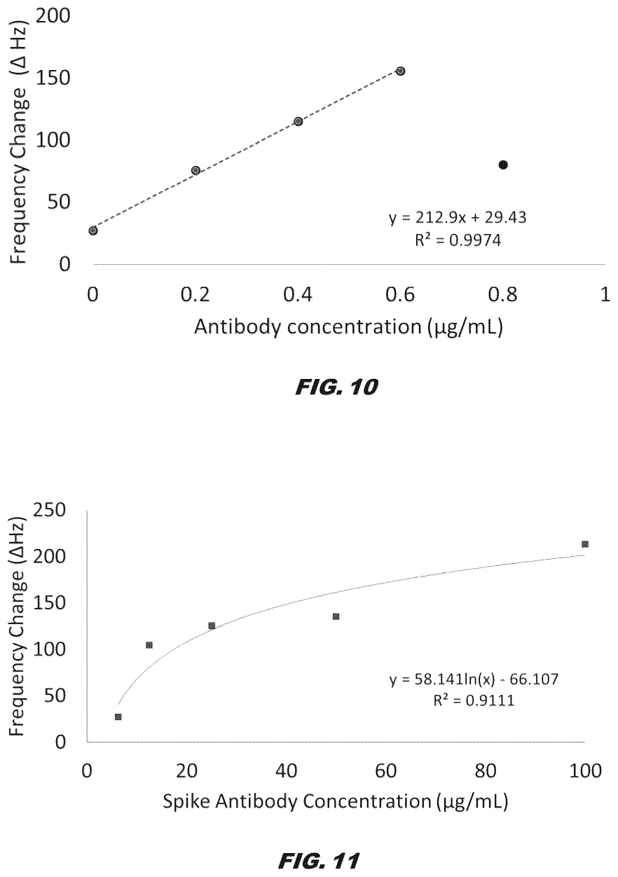

FIG. 10 shows Version 1-In PBS, using 100% 11-MUA as the initial alkanethiol layer (SAM), the dynamic range of the sensor was ~0-0.8 μg/ml.
100% 11-MUA as alkanethiol layer
Spike antibody (Ab) was added to PBS 0-0.8 μg/mL concentrations
At higher concentrations, response seems to decline or saturate.

FIG. 11 shows Version 2-In PBS, using 50% 11-MUA and 50% 9-Mercapto-1-nonanol as the initial alkanethiol layer (SAM), the dynamic range of the sensor increased to ~0-100 μg/ml.
100% 11-MUA as alkanethiol layer
Spike antibody (Ab) was added to PBS 0-0.8 μg/mL concentrations
At higher concentrations, response seems to decline or saturate.

FIG. 12 shows Testing sensor with spike antibody in calf blood plasma diluted in PBS

Version 3-However, in calf blood plasma, using 20% 11-MUA and 80% 9-Mercapto-1-nonanol as the initial alkanethiol layer (SAM), the dynamic range of the sensor decreased to ~0.625-3 μg/ml.
20% MUA/80% nonanol as alkanethiol layer
Spike antibody (Ab) was added to Calf Blood Plasma and serially diluted in PBS to give 0.625-10 μg/mL concentrations
At higher concentrations, response seems to decline, but overall dynamic range seems similar to 100% MUA (QCM1).
At lower concentrations, response is non-linear

FIG. 13 shows raw data from our most recent sensor using the above immobilization chemistry on a conventional 10 MHz quartz resonator shows high sensitivity in measuring 12-100 ng/ml of anti-S IgG antibody in 100 μl of plasma from calf blood. Sensitivity analysis of the above Acousto-Ab sensors using their responses to different concentrations of anti-S IgG antibody shows a sensitivity of 1.74 Hz/ng/ml. There was a measurable response of 48.3 Hz to non-specific binding of plasma proteins (in calf blood) on the surface of the sensor. A drop of blood from a finger stick (~50 μl) is estimated to contain 362 ng & 882 ng of IgM and IgG, respectively, which is far larger than the values tested in the above experiments. Therefore, preliminary experiments indicate that the sensitivity of the current Acousto-Ab system appears to be more than adequate for human blood samples.

Results of sensitivity analysis show the sensor demonstrating a linear response to increasing concentrations of anti-S IgG in plasma from calf-blood. (Inset) Typical time series responses of the sensor (change in frequency) to 4 different concentrations of anti-S IgG in plasma from calf blood. A response of ~48.3 Hz at 0 ng/ml indicates the response of the Acousto-Ab sensor to non-specific binding of plasma proteins in calf blood.

Multi-channel resonators. FIG. 14 shows a photograph of a two channel quartz resonators fabricated on a glass substrate. Scale bar=300 mm. Data published in Khraiche and Muthuswamy, Lab on Chip, 2012, 12, 2930-2941.

Protein spotting on micro-scale sensors. New capabilities developed in the lab of Dr. Khraiche at American University of Beirut in Lebanon that help us realize precision spotting of proteins on micro-scale electrodes. FIG. 15 shows an image of Example of fluid spotting precision on 30 μm diameter electrodes.

### Scheme for Blood Sample Collection

FIG. 16 shows Illustration of blood being sampled by a lancet pen and dispensed into a dilution well (filled with PBS). It is then filtered using a graphene filter before releasing plasma into the sensor well in the cartridge.

### Example 4

Serological testing for antibodies to SARS-COV-2 (Severe Acute Respiratory Stress-Coronavirus-2) or other viruses that can be pandemic, is critical for a variety of important purposes. These include critical assessment of immunity levels, tracking the efficacy of vaccines during their development process and after vaccine administration, seroprevalence, and plasma evaluation.

This example addresses the need for point of care, rapid, and quantitative antibody readout among immune-compromised individuals, vaccine developers, doctor's offices, and blood banks that screen and evaluate plasma for convalescent therapy. The serological tests that are EUA (emergency use authorization from FDA) approved do not meet the needs of being both point of care and quantitative. Without wishing to be bound be theory, we can develop a point-of-care, rapid, quantitative in vitro diagnostic (IVD) with high sensitivity, specificity, and scalability based on innovations in acoustic. The IVD will address current challenges in precise quantitation, access, availability, deployment, and communication of results, ease of use, and scalability to large and remote populations.

Acousto-Ab series, will be a portable point-of-care IVD device consisting of a cell phone-sized measurement unit and replaceable cartridges. The device will quantitatively assess IgG, IgM and IgA antibodies separately on different platforms or sequentially on the same platform for the SARS-Cov-2 virus rapidly (~5 min) in a droplet of blood from a finger stick, with high sensitivity and specificity. A multi-channel version of Acousto-Ab will simultaneously assess antibodies to Spike(S), Nucleocapsid (N) and NSP5 proteins that will enable the detection of antibodies to emerging variants also. The system will be designed to be quickly deployed and as easy to use as a home blood glucose meter and will communicate results back to health care providers through a mobile phone or other hand-held computing platforms. The system can be readily adapted in future modifications to track over 30 or more biomarkers simultaneously. Comparable systems available in the marketplace today that use enzyme-linked immunosorbent assays (ELISA) require trained personnel, extensive instrumentation and often take multiple hours between sample collection and reporting of test results. In addition, current FDA-approved point-of-care systems for serological tests provide only a qualitative assessment of antibodies using lateral flow assays.

### Background and Significance

Recent developments in serological testing for antibodies to SARS-COV-2 (Severe Acute Respiratory Stress-Coronavirus-2) are moving towards addressing the critical need for a point-of-care IVD capable of simultaneously providing rapid and quantitative readouts of antibodies [3-7]. A recent study on a limited number of patients who were seropositive for COVID-19 reported median IgG antibody titers in the range 0.29-1.38 μg/ml depending on the number of symptoms (0-5) exhibited by the patients [8]. The minimum and maximum antibody titers ranged from 0.001-0.746 μg/ml and 5.45-11.12 μg/ml, respectively, depending on the number of symptoms (0 to 5 symptoms) [8]. Quantitative assays are therefore essential for state and federal health agencies to determine the seroprevalence and thus immunity levels of the target community, and to better understand the immune mechanisms for example, in immune compromised individuals. For instance, concentrations of antibodies in blood to spike protein of SARS-COV-2 were found to be either unaltered post-vaccination or significantly lowered post-vaccination in immune-compromised patients than those of normal people [9]. For vaccine developers, it is crucial to obtain a rapid, quantitative readout of levels of antibodies in the blood samples to track their production rate immediately after vaccine administration and subsequent decay in the following months. A point-of-care, rapid, quantitative assay will ease the compliance burden on the patients involved in clinical trials by not requiring them to repeatedly report to a central laboratory.

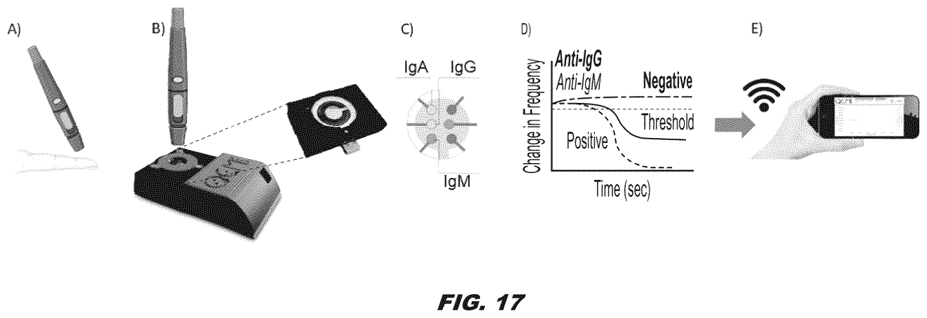

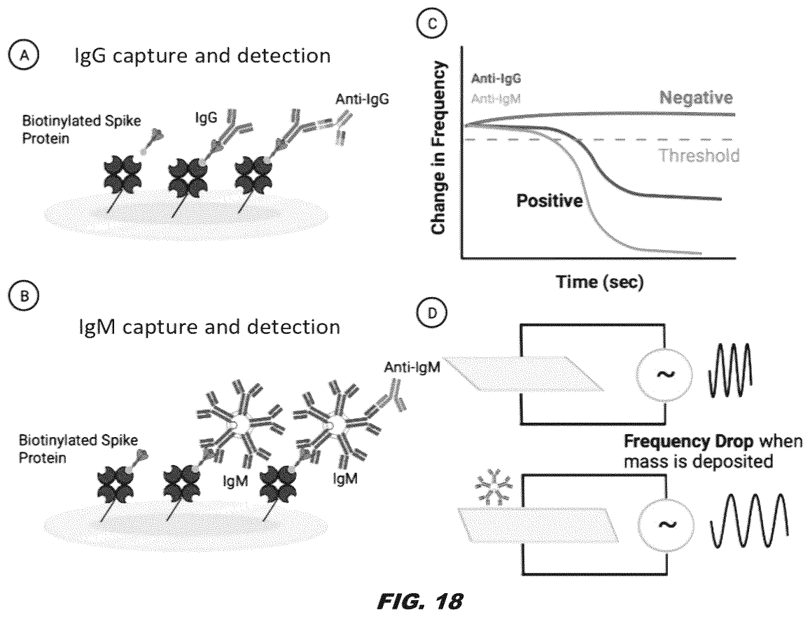

Herein, we describe a new, hand-held, point-of-care, rapid quantitative assay, which we call Acousto-Ab, to detect IgG, IgM and IgA antibodies for the SARS-Cov-2 virus, respectively, with high sensitivity and specificity in blood samples to track immunity levels. The approach uses a new gravimetric detection system for antibodies to SARS-Cov-2 proteins that relies on changes in the resonant frequency of an acoustic resonator in response to binding events between the immobilized antigen (receptor-binding domain of the spike S-protein or other sub-units of the S-protein or the nucleocapsid N-protein from SARS-COV-2) on the surface of the sensor and the antibody that needs to be detected. Such molecular binding events cause changes in mass adhered on the resonator and viscosity of the resonator-liquid interface. We will track energy dissipation changes in addition to changes in resonant frequency of the resonator to simultaneously track mass and viscosity changes in the interface. The overall concept is illustrated in FIG. 17 and the sensing principle is illustrated in FIG. 18. The gravimetric approach using resonators will allow our platform to integrate sensing of multiple biomarkers from the same blood sample in a single sensor cartridge. The approach will go a long way to future-proof the technology to new discoveries of immune biomarkers for COVID19, resulting in new emerging point-of-care disease screening markets.

Herein centers on physical and chemical methods to track changes in the resonant frequencies (in response to biomolecular binding events) of multiple millimeter- or micrometer-scale acoustic resonators on a single substrate reliably and simultaneously while operating in a highly viscous liquid medium such as blood. This allows label-free detection from one sample without the need for microfluidics.

The effort is a systematic application of our prior knowledge and experience designing and developing acoustic resonators platforms for sensing neurotransmitter γ-aminobutyric acid (GABA) & anti-GABA, and also monitoring the dynamics of cell adhesion [10-15]. We will use this experience to develop our prototype Acousto-Ab point-of-care diagnostic and systematically optimize the sensitivity, cross-reactivity or specificity, and stability.

Work measuring anti-GABA using commercially-available acoustic resonators demonstrated a detection limit of 10 nM and a change in frequency of 500 Hz for 10 μg/ml of anti-GABA [14, 15]. A virtue of the proposed sensing platform is that the sensitivity of the acoustic resonator operating under viscous fluids can be further optimized for resonant frequency and the dimensions of the sensing area, as we have demonstrated in our studies [11, 12]. We have shown that we can achieve increases in sensitivity of over seven orders of magnitude by increasing the resonant frequency (up to 60 MHz) and the dimensions of the sensing area of the electrode (minimum diameter of 30 μm). In phase I of this project proposal, we will focus on the deployment of the α-prototype in a commercial laboratory that meets the FDA's requirement for sensitivity, specificity, and stability for LDT approval to achieve business feasibility. Subsequently, we will file point-of-care FDA Class II or EUA in phase II whichever meets our business needs.

Project 1—We will design, develop and test an α-prototype of Acousto-Ab system to detect antibodies to a S-protein in human blood samples and obtain early customer feedback.

Rationale—We have demonstrated that the approach will work for antibodies to neurotransmitter GABA and in sensing purified samples of monoclonal IgG antibodies to the N-protein in PBS and S1-protein of SARS-COV-2 in calf serum. The method will be validated in sero-positive human blood samples with IgG, IgM and IgA levels typically observed in COVID-19 patients in the weeks following infection. The acoustic sensors can be optimized for dynamic range and sensitivity to perform under varying levels of viscosity typically seen in blood, sample volumes, and sample injection methods. The temporal levels of different classes of antibodies (IgM, IgG, IgA) in blood are a powerful biomarker panel that can be used to understand temporal progression of pathology and can help to both classify disease stage and severity and focus treatment approaches.

IgM can be an indicator of early immune response, after which class-switching is observed. Additionally, in human Covid-19 pathology, IgA is a potent, neutralizing antibody that plays a crucial role in the immune response, especially toward viral and other pathogens. Although it can be found in mucosal linings, IgA is the second-most abundant antibody in serum and has a proinflammatory role in activating astrocytes and myeloid lineage cells that include macrophages, monocytes, microglia, neutrophils. However, recent studies show that high IgA levels correspond to disease severity in Covid-19 patients and are expressed persistently in serum up to 365 days post infection [16, 17]. Serum IgA is 3-fold higher in patients with severe COVID In human Covid-19 pathology, IgA is a potent, neutralizing antibody that plays a crucial role in the immune response, for example toward viral and other pathogens.

We will integrate 10 and 25 MHz quartz crystal resonators with a sandwich assay involving biotinylated S-protein or N-protein from SARS-COV-2 immobilized on the sensor surface as shown in FIG. 18. The test serum samples will have unknown quantities of IgG, IgM or IgA antibodies and will be mixed with known amounts of human anti-IgG or human anti-IgM or human anti-IgA to detect IgG, IgM or IgA respectively. We will utilize proven immobilization methods, illustrated in FIG. 18, to produce stable anchoring of bioactive antigen molecules with reproducible orientations to ensure consistent coating on the gold electrodes (yellow oval in FIG. 18) on the quartz resonator (blue oval in FIG. 18). In embodiments, commercially available biotinylated SAM will be applied to the gold surface. This is followed by the attachment of streptavidin and then biotinylated sub-units or receptor-binding domain (RBD) of S-protein or N-protein. The N-protein (amino acid residues 1-419, Genbank accession QHD43423.2), the spike protein (Genbank accession QHD43416.1), S1 domain composed of amino acid residues 16-690, the S2 domain (amino acid residues 698-1213), and the RBD region of the S1 (amino acid residues 319-541), will be tested as antigens. We will use protein BLAST analysis (blast.ncbi.nlm.nih.gov/Blast) to determine the sequence homologies of N and S proteins to the corresponding protein sequences from other SARS-COV-2 isolates deposited in the GenBank database (taxonomy id 694009), and to try to achieve >98% homology. We will also try to minimize the sequence homology to the respective N and S proteins from four human coronaviruses (strains OC43, HKU1, 229E, and NL63) that cause common cold symptoms to minimize interferents and enhance specificity. The Human SARS-COV-2 Serology Standard (a pool of plasma from four donors) will be obtained from Fredrick National laboratory for cancer research for final testing. Alternatively, we will buy commercially available human blood samples (Covid +/−) from Innovative Research Inc. (Novi, MI).

Filtering blood samples—The disposable cartridge housing the sensor will have a sealed well into which a drop (~50 μl) of blood sample will be dispensed as illustrated in FIG. 16 using a commercially available blood lancet pen. The sealed well will contain heparinized dilution buffer (10×dilution factor) and a commercial graphene filter at the bottom to separate plasma from cells. Studies in our lab indicate the 10×dilution factor allows for (1) detection of anti-S protein antibody levels within the published dynamic range for human pathology and (2) minimizes non-specific binding effects on the sensor. The diluted plasma with the anti-S antibodies will then be filtered into the QCM-sensor well in the disposable cartridge via gravity flow (~100 μl volume) and tested for virus-antibody binding events in both single and multi-channel Acousto-Ab platforms.

Finally, the sensor will be topped with 20 μg/ml of anti-human IgG or IgM or IgA (depending on whether we are sensing IgG, IgM or IgA respectively) that will elicit a change in resonant frequency proportional to the concentration of IgG, IgM or IgA from the sample that is already bound to the sensor. Alternatively, all three (anti-human IgG, IgM and IgA) will be added in sequence on the same sensor to give a sequential read-out of IgG, IgM and IgA respectively. Each stage will be monitored via response of the Acousto-Ab sensor, atomic force microscopy (AFM), and X-ray photoelectron spectroscopy (XPS) available at ASU for industrial users for a service fee. We will validate various concentrations of IgG/IgM/IgA (1×10⁻⁴-100 μg/ml) and build dose-response curves with change in resonant frequency plotted as a function of antibody concentration (FIG. 13). We will use commercial open-QCM systems during the initial prototyping phase to obtain rapid customer feedback.

Sensitivity to antibodies: In embodiments, the sensor will measure anti-IgG (or anti-IgM or anti-IgA) binding to any IgG (or IgM or IgA) from the serum sample once the antibodies bind to the immobilized S-protein on the sensor surface. Raw data (in FIG. 13) from our most recent Acousto-Ab sensor using the immobilization chemistry described herein on a conventional 10 MHz quartz resonator shows high sensitivity in measuring 12-100 ng/ml of anti-S IgG antibody in 100 μl of plasma from calf blood. Sensitivity analysis of the above Acousto-Ab sensors using their responses to different concentrations of anti-S IgG antibody shows a sensitivity of 1.74 Hz/ng/ml. There was a measurable response of 48.3 Hz to non-specific binding of plasma proteins (in calf blood) on the surface of the sensor. A drop of blood from a finger stick (~50 μl) is estimated to contain 362 ng & 882 ng of IgM and IgG, respectively, which is far larger than the values tested in other experiments described herein. Therefore, experiments indicate that the sensitivity of the current Acoust-Ab system appears to be more than adequate for human blood samples. Typical half-life (in days) and serum levels of IgG, IgM and IgA in humans are summarized in Table 4. A recent study reported the antibody-titers of 120 seropositive individuals ranged 0.001-11 μg/ml [8].

TABLE 4
Comparison of IgG, IgM & IgA in Normal Human Serum

| | | Typical | |
|---|---|---|---|
| | | Half- | |
| | Approximate | life in | Typical Serum |
| Antibody | Molecular | Blood | Levels (mg/ml) |
| Type | Weight (g/mol) | (days) | *age-dependent |
| IgG | ~150,000 | ~25 | 3.5-47 |
| IgM | ~900,000 | ~5-6 | 0.5-2.0 |
| (pen- | | | |
| tamer) | | | |
| IgA | ~160,000 | ~4-6 | 2-3 |
| | (monomer, serum) | | |
| | ~320,000 | | |
| | (dimer, secretory, mucosa) | | |

We will perform expanded versions of such sensitivity analyses to determine calibration curves for Acousto-Ab for a wide range of concentrations of IgG, IgM and IgA antibodies to the RBD domain of the spike protein (S-protein) in plasma. Under experimental conditions, these calibration curves will be used to determine the concentrations of IgG, IgM and IgA in plasma sample from the measured responses of Acousto-Ab. Replicates of 5 each for ten distinct concentrations of IgG, IgM and IgA, at an α=0.05, is expected to result in a statistical power of 0.89. IgG can be found in monomeric form, whereas IgM can be found in pentamers, which is reflected in the 6-fold relative change in molecular weight (Table 4). This change is expected to result in a higher sensitivity.

Testing for cross—reactivity or specificity-We will validate the serologic cross-reactivity of the diagnostic platform for human IgG, IgM, IgA antibodies to available anti-human IgG, IgM, and IgA.

Power analysis—For n=5 trials, and a of 0.05, 10% variability and a difference in means of 20%, the statistical power of the above comparison is estimated to be 0.89.

The FDA also recommends testing for cross-reactivity against the following antibodies—anti-influenza A (IgG and IgM), anti-influenza B (IgG and IgM), anti-HCV (IgG and IgM), anti-HBV (IgG and IgM), anti-Haemophilus influenza (IgG and IgM), anti-229E (alpha coronavirus), anti-NL63 (alpha coronavirus), anti-OC43 (beta coronavirus), anti-HKU1 (beta coronavirus), ANA, anti-respiratory syncytial virus (IgG and IgM) and anti-HIV. We will test a minimum of 5 individual samples for each disease/infectious agent/antibody class listed above (as per FDA recommendation). In addition, we will prepare plasma samples spiked with the IgM or IgG or IgA antibodies for the underlying conditions. We will use commercially-available IgM or IgG antibodies panels for the underlying conditions collected before the COVID-19 pandemic to ensure the panels are SARS-COV-2 antibody negative.

Class-specificity testing-Since our Acousto-Ab devices will quantitatively assess the different classes of immunoglobulins, we will perform the following class-specificity testing as recommended by the FDA. We will use the dithiothreitol (DTT) assay on all configurations of the Acousto-Ab diagnostic systems to assess their class-specificity, where the signal due to IgM either decreases or becomes negative upon application of DTT but the signal due to IgG remains unaffected. We will test the Acousto-Ab systems with five samples each of (IgG/IgM, +/+), (IgG/IgA, +/+), (IgA/IgM, +/+) and two replicates each (as recommended by the FDA template). We expect to see 100% agreement between the results of the diagnostic systems and the expected outcome of DTT treatment (—/+) to IgM/IgG, +/+. To confirm DTT activity, a positive control test will also be included.

### Milestone 1—Completion of α-Prototype of Acousto-Ab Platform.

Project 2—we Will Optimize and Test β-Prototypes of Acousto-Ab Platform for S- and N-Proteins Based on Early Customer Feedback from Our α-Prototypes.

Based on our reading of the literature and user interviews, without wishing to be bound by theory, we will see the following—(1) improvement in sensitivity and detection limit to match those of ELISA with a detection limit of 0.1 ng/ml and sensitivity of at least 1-5 Hz/ng/ml for IgG, IgM and IgA to S-protein and N-protein in blood samples. (2) expand the repertoire of Acousto-Ab to assess IgG, IgM and IgA to S- & N-protein of SARS-Cov-2.

Enhanced sensitivities—Without wishing to be bound by theory, the calculated sensitivity of the commercial crystals can sense IgG, IgM and IgA from COVID-19 positive patients, as indicated by our estimates herein. However, estimates from seropositive patients indicate antibody titers as low as one ng/ml in asymptomatic patients⁷. If lower detection limits emerge as being important in our customer interviews, we will use the techniques outlined previously¹³ to validate electrode diameters in the range of 50 μm-5 mm and resonant frequencies (10, 25, and 60 MHz) to enhance sensitivities and detection limits. The sensitivity of the resonator is directly proportional to the square of electrode diameter if the resonator is operating in the air, as given by the classic Sauerbrey equation. Another way to explain this electrode diameter-sensitivity relationship is through an improvement in the Q-factor of the resonator that is caused by a decrease in electrical resistance of the electrodes as its diameter increases. However, when the resonators operate in liquid, particularly in a viscous liquid such as blood or plasma (at room temperature), the viscous interface on the electrode adds further resistance to the crystal resulting in dampening of oscillations, decrease in Q-factor, and loss of sensitivity. We will use the theoretical equations and experimental results outlined in our most recent publication to achieve up to seven orders of magnitude increase in sensitivity to counter any loss of sensitivity due to the viscosity of blood.

We will purchase blank quartz crystals from Xeco Inc., Cedar City, UT, USA and use ink-jet printing to pattern electrodes on the surface of the crystal. The printing process will be done for a service fee via an ink-jet printer (DMP-2850, Fujifilm Dimatix, USA) fitted with a piezo-driven 16-nozzle print-head available in the lab of MK in AUB. The conductive patterns and coatings will be printed using a 1 pL cartridge, while the passivation layers will be printed using a 10 pL cartridge.

Expanded repertoire of antibody sensing-Current antibody assays for COVID-19 usually detect anti-S protein or anti-N protein antibodies since the S- and N-proteins are considered highly immunogenic [5, 19]. Therefore, in a separate diagnostic, we will expand the repertoire of Acousto-Ab platform to sense IgG, IgM & IgA antibodies to the N-protein.

If a significant false-positive rate (>5%) is observed, we will validate different anti-human-IgG, anti-human-IgM and anti-human IgA to identify candidates that minimize response of the sensor to cross-reactivity. We will also validate alternate antigen sequences with lower homologies to other human antibodies that are potential cross-reactants.

Testing and improving the stability of the sensing cartridge-We will validate methods to extend the shelf life of the sensing cartridge at room temperature via stabilizing the surface-bound S1-protein or N-protein.

Rationale—Deploying embodiments described herein in the field requires accounting for conditions that may affect the stability and lifetime of the anti-IgG, anti-IgM and anti-IgA in the sensing cartridge.

Experimental Design—The process for adding stabilizers will consist of covering the surface with (a) 0.15 M blocking buffer (Thermo Fisher Scientific, USA), (b) 5% w/v sucrose (catalog #AC177142500, Acros organics), and (c) coating stabilizer and blocking buffer diluted 1:1 with water. The devices will then be aspirated and dried. All devices will be stored at 50° C. to age the coating (1 day at 50° C. is equal to 6.5 days at room temperature). We will assess the stability of the coating at ten days, three weeks, and three months. We will attempt to achieve an 18-month shelf life at room temperature. Besides the in-use stability assessment of reagents in the above experiments, we will also assess the stability of the reagents under the following conditions:

Shelf-life stability—Reagent shelf life will be assessed by real-time stability testing, with reagents stored at the specified storage temperature.

Stress testing—The reagents will be cycled through temperatures of 4° C. and ambient temperature to mimic shipping conditions. A separate cohort of reagents will be cycled through different light conditions to simulate shipping conditions. They will then be placed under normal storage conditions and their in-use stability assessed.

outcomes—Without wishing to be bound by theory, samples covered by sucrose will have the highest stability.

Milestone 2—Completion of α-prototype.

Milestone 3—Completion of cross-reactivity assessments for the α- & β-prototypes.

We will scale up the Acousto-Ab platforms to simultaneously sense more than one antibody from a single sample.

Rationale—A unique innovation of our system is to do multiplexed label-free assessment of antibodies from a single sample using multiple resonators fabricated on the same substrate (avoiding the need for fluidics). As new variants of the SARS-Cov-2 emerge, it is important to have a quantitative profile of antibodies to the different sub-domains of the S-protein and other viral proteins. Such multi-dimensional profiling of blood biomarkers of immunity will not only be necessary to assess efficacies of neutralizing antibodies but also to assess the level of immunity and to track infection progression and severity of disease. Recent microarray studies¹⁹ have also identified antibodies to other viral proteins such ORF9b and NSP5 as being activated in seropositive patients. Therefore, in addition to S-, N- and the RBD domain of S-proteins, we will validate the capability of the diagnostic to simultaneously measure NSP5 antibodies to assess the generalizability of the diagnostic platform.

Sensor design—Electrodes (sensing area) corresponding to multiple sensing sites will be spaced at least one electrode diameter apart to minimize interference in the acoustic waves between neighboring sites. In our prior studies, we have successfully demonstrated the use of multiple microscale acoustic sensors on the same quartz substrate to sense neuronal activity over a period of 9 days in culture, as shown in FIG. 14 [11]. The diameter and thickness of each electrode (along with sensor frequency) will determine final sensitivity. As in previous sections, we will use inkjet printing to build our electrodes (sensing area) on both sides of the quartz substrate. (FIG. 14).

Chemistry—We can pattern biomolecules on these microscale electrodes of gold and conductive polymers. Therefore, we can use that capability to pattern different proteins and their sub-units (S1-protein, N-protein, RBD domain of the spike protein, or NSP5) on each of the microscale electrodes using the same immobilization chemistry outlined in FIG. 18 except that the different proteins (S1, nucleocapsid (N), and the NSP5) will be spot printed on separate electrodes sequentially (as shown in FIG. 15). We can use a drop-on demand system (FluidFM BOT, Switzerland) which can be used for manipulating cells, that our team modified for liquid spotting, that allows for precision control when adding liquids to the surface.

Testing—We will add plasma samples directly to the sensor cartridge housing the quartz substrate with multiple sensors. After allowing for equilibration, we will add anti-human IgG or IgM or IgA depending on whether we are detecting IgG, IgM or IgA respectively.

Completion of multi-channel prototype of Acousto-Ab platform.

The relative proportions of IgM/IgG/IgA will also be validated by different fluorescently bound secondary antibodies. In the event, we don't achieve desired specificity with anti-human IgG, IgM and IgA, we will attempt to create a response profile of frequency changes at harmonics of the resonant frequency that will help distinguish between IgG/IgM/IgA due to differences in binding kinetics to antigen. We will monitor the above profile of frequency changes in response to a combinatorial application of different Ig-specific proteases such as Igase (specific to IgA), IdeZ (specific to IgG), serine proteases (specific to IgM). As the different proteases cleave their respective Ig, the profile of frequency changes in response to those events will determine the different fractions of IgG/IgM/IgA bound to the viral protein on the surface of the sensor. A second alternative is to use Protein A or G (binds to the Fc domain of IgG) to characterize the presence of IgG and jacalin (an IgA specific lectin) to characterize the presence of IgA and its corresponding change in resonant frequency of the resonator.

Testing with serological samples-We will test retrospectively-collected SARS-CoV-2 antibody-positive specimens from patients that have been previously confirmed infected by SARS-COV-2 RT-PCR tests. These specimens will be purchased. The specimens will be accompanied by basic information such as the population from which the sample was drawn and the comparator method, specimen collection date, date of onset of symptoms (if present/known), and comparator method to confirm infection with SARS-COV-2.

### REFERENCES CITED IN THIS EXAMPLE

1. Lee, C. Y. P., et al., Serological Approaches for COVID-19: Epidemiologic Perspective on Surveillance and Control. Frontiers in Immunology, 2020. 11 (April): p. 1--7.
2. Harpaz, R., R. M. Dahl, and K. L. Dooling, Prevalence of Immunosuppression Among US Adults, 2013. JAMA, 2016. 316 (23): p. 2547-2548.
3. Bastos, M. L., et al., Diagnostic accuracy of serological tests for covid-19: Systematic review and meta-analysis. The BMJ, 2020. 370.
4. Ainsworth, M., et al., Performance characteristics of five immunoassays for SARS-COV-2: a head-to-head benchmark comparison. The Lancet Infectious Diseases, 2020. 20 (12): p. 1390--1400.
5. Grzelak, L., et al., A comparison of four serological assays for detecting anti-SARS-COV-2 antibodies in human serum samples from different populations. Science Translational Medicine, 2020. 12 (559).
6. Li, Z., et al., Development and clinical application of a rapid IgM-IgG combined antibody test for SARS-COV-2 infection diagnosis. Journal of Medical Virology, 2020. 92 (9): p. 1518--1524.
7. Liu, G. and J. F. Rusling, COVID-19 Antibody Tests and Their Limitations. ACS Sensors, 2021. 6 (3): p. 593--612.
8. Bartsch, Y. C., et al., Discrete SARS-COV-2 antibody titers track with functional humoral stability. Nature Communications, 2021. 12 (1).
9. Hagin, D., et al., Immunogenicity of Pfizer-BioNTech COVID-19 vaccine in patients with inborn errors of immunity. J Allergy Clin Immunol, 2021. 148 (3): p. 739-749.
10. Zhou, A. and J. Muthuswamy, Acoustic biosensor for monitoring antibody immobilization and neurotransmitter GABA in real-time. Sensors and Actuators B: Chemical, 2004. 101 (1): p. 8--19.
11. Khraiche, M. and J. Muthuswamy, Multi-modal biochip for simultaneous, real-time measurement of adhesion and electrical activity of neurons in culture. Lab on a Chip, 2012.
12. Khraiche, M. L., J. Rogul, and J. Muthuswamy, Design and development of microscale thickness shear mode (TSM) resonators for sensing neuronal adhesion. Frontiers in Neuroscience, 2019.
13. Khraiche, M. L., A. Zhou, and J. Muthuswamy, Acoustic sensor for monitoring adhesion of Neuro-2A cells in real-time. Journal of Neuroscience Methods, 2005.
14. Wang, T., et al., Immobilization and characterization of gamma-aminobutyric acid on gold surface. J Biomed Mater Res A, 2006. 79 (1): p. 201--209.
15. Wang, T. and J. Muthuswamy, Immunosensor for detection of inhibitory neurotransmitter gamma-aminobutyric acid using quartz crystal microbalance. Anal Chem, 2008. 80 (22): p. 8576--8582.
16. Dobano, C., et al., Persistence and baseline determinants of seropositivity and reinfection rates in health care workers up to 12.5 months after COVID-19. BMC Med, 2021. 19 (1): p. 155.
17. Kulikowska, J., et al., The Significance of COVID-19 Immunological Status in Severe Neurological Complications and Multiple Sclerosis-A Literature Review. Int J Mol Sci, 2021. 22 (11).
18. Ivanov, A. and E. Semenova, Long-term monitoring of the development and extinction of IgA and IgG responses to SARS-COV-2 infection. J Med Virol, 2021. 93 (10): p. 5953-5960.
19. Jiang, H. W., et al., SARS-COV-2 proteome microarray for global profiling of COVID-19 specific IgG and IgM responses. Nat Commun, 2020. 11 (1): p. 3581.

### Example 5

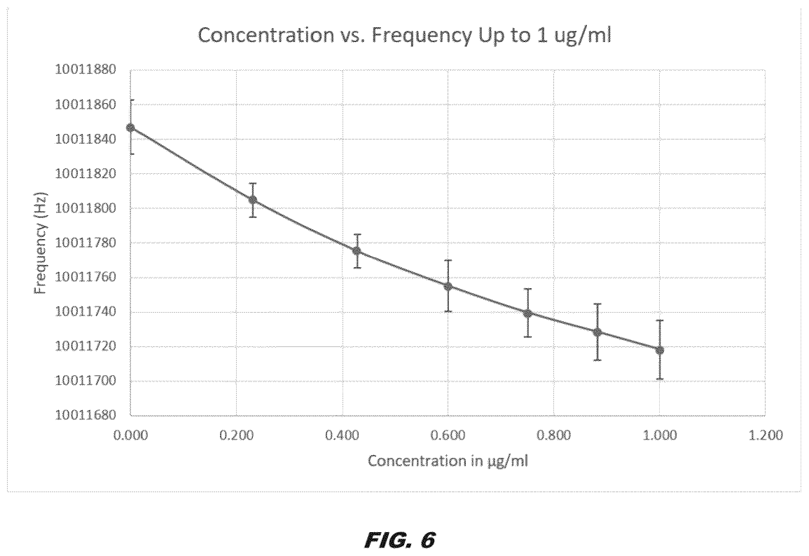

Commercially available, 10 MHz crystals (14 mm diameter) with gold electrodes (QCM Open, Novaetech™ Srl) were sensitized with spike receptor binding domain (RBD) protein as the sensing element using carboiimide (EDC-NHS) chemistry (FIG. 7) and streptavidin-biotin linkage (FIG. 8). The QCM was placed in 20 mM 11-mercaptoundecanoic acid (11-MUA, Chemcruz™) in absolute ethanol for at least 24 hours at room temperature (23° C.). Next the 11-MUA treated QCM was incubated with 1:1 ratio (10 mg/ml) of 1-ethyl-3-(3-dimethylaminopropyl) carboiimide hydrochloride (EDC-HCL) and N-hydroxysuccinimide (NHS) in 0.1 M 4-morpholineethanesulfonic acid (MES) for 1 hour and washed once with MES buffer, followed by streptavidin (Millipore™) (200 μg/ml) in MES buffer for 1 hour. The streptavidin-immobilized surface was washed thrice in PBS (3 min each) followed by 1 M ethanolamine in phosphate buffered saline (0.01 M PBS, pH 8) to remove unreacted NHS for 30 min. After washing again in PBS thrice, the streptavidin immobilized QCM surface was incubated with 2 μg/ml of biotinylated-spike protein (Sino Biological, catalog number 40592-V27H-B) overnight. After washing in PBS, the QCM-Spike-Sensor was blocked in diluted calf-blood serum (1:10) overnight (~8 hours) to minimize non-specific adsorption and washed once in PBS prior to use.

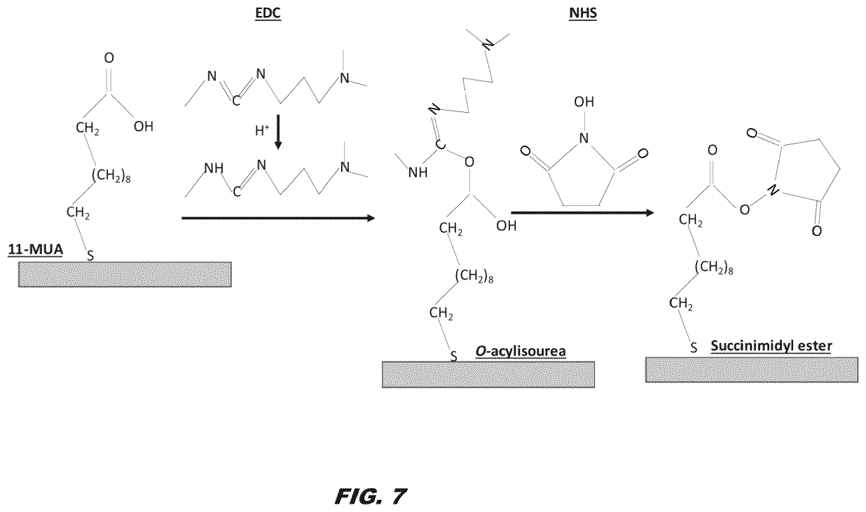

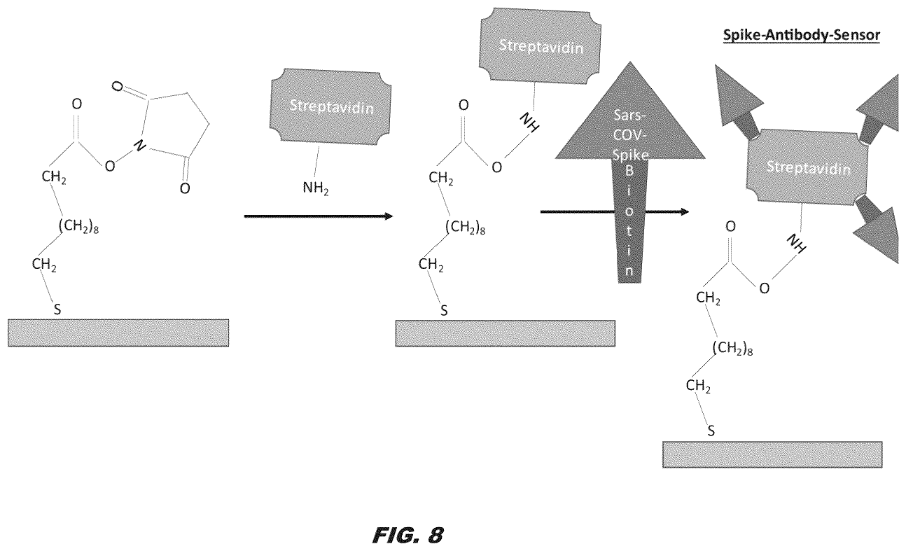

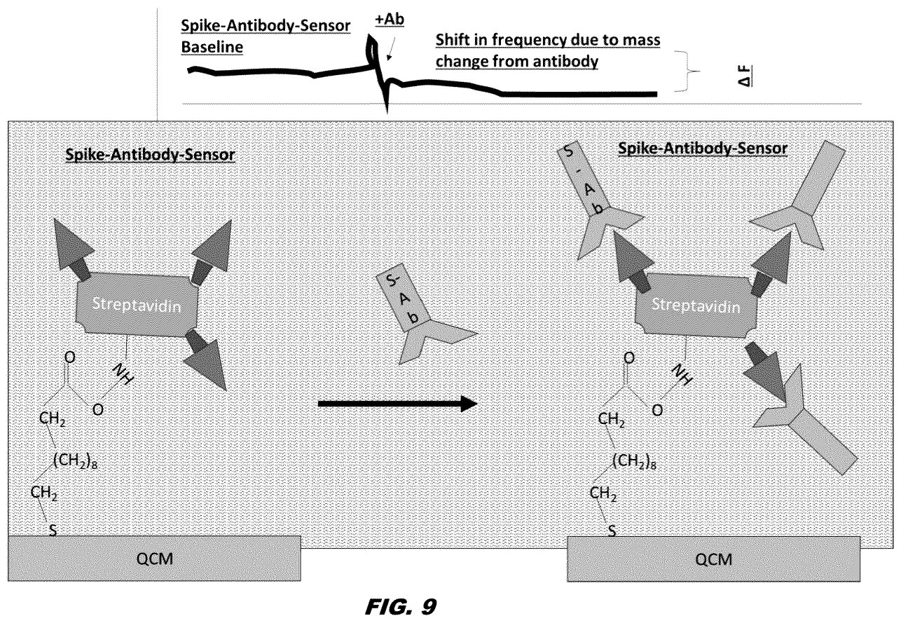

(FIG. 9) For calibration, IgG-Spike was serially diluted (1 μg/ml, 100 ng/ml, 50 ng/ml, 25 ng/ml, 12.5 ng/ml) in diluted calf blood serum isolated by centrifuging heparinized, whole calf blood (Rockland,inc.™) for 60 min at approximately 1000×g, followed by dilution (1:10) in PBS. The QCM-Spike-Sensors were placed in the OpenQCM module and calibrated to their resonance frequency (~10 MHz) in 100 μl of PBS. Sensorograms were generated by manually pipetting in and replacing solutions (100 μl) in the sensor well every 5 min.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

1. A diagnostic platform for detection of at least one biomarker in a fluid sample comprising:
a quantitative sensing device configured to receive the fluid sample, wherein the fluid sample comprises blood, blood serum, or plasma;
an electronic reading platform; and
a computing device,
wherein the sensing device comprises:
a layer of piezoelectric material comprising two faces;
at least one electrode layer, wherein the at least one electrode layer is affixed to one face of the piezoelectric material, wherein the at least one electrode layer comprises:
a thickness of about 33 nm to about 300 nm, and
a diameter of about 150 μm to about 350 μm;
a second reference electrode layer that is affixed to a second face of the piezoelectric material;
one disposable sensor cartridge, wherein the cartridge comprises a means for detecting IgG, IgM, IgA antibodies, or a combination thereof, and wherein the IgG, IgM, IgA antibodies, or a combination thereof are quantitively detected;
a Q-factor of at least 10,000;
an operating frequency of about 10 MHz to about 90 MHz; and
a sensing layer disposed upon the at least one electrode layer, wherein the sensing layer is configured to bind the at least one biomarker for a disease or condition, wherein the biomarker comprises an IgG antibody, an IgM antibody, an IgA antibody, or a combination thereof;
wherein the quantitative sensing device is communicatively linked with the electronic reading platform,
wherein the electronic reading platform is configured to receive sensor data from the sensing device and to communicate the data to the computing device, and
wherein the computing device is configured to determine the presence, absence, or amount of the at least one biomarker; and
wherein the diagnostic platform quantitatively detects the at least one biomarker within about 10 minutes.

2. The diagnostic platform of claim 1, further comprising a fingerstick system configured to obtain the blood from a patient.

3. The diagnostic platform of claim 1, wherein the diagnostic platform comprises a portable, hand-held device.

4. The diagnostic platform of claim 3, wherein the portable, hand-held device is configured to be worn by a user.

5. The diagnostic platform of claim 1, wherein the diagnostic platform is configured to determine the presence, absence, or amount of the at least one biomarker within about 10 minutes after the fluid sample contacts the sensing device.

6. The diagnostic platform of claim 1, wherein the piezoelectric material comprises a quartz crystal, PZT (lead zirconate titanate), lead titanate, Barium titanate, Zinc Oxide, lead magnesium niobate lead titanate (PMNPT), polyvinylidene difluoride, polyvinylidene fluoride (PVDF), Aluminum nitride, Gallium nitride, or a combination thereof.

7. The diagnostic platform of claim 1, wherein the piezoelectric material comprises a diameter of up to about 153 mm.

8. The diagnostic platform of claim 1, wherein the piezoelectric material comprises a thickness of up to about 3 mm.

9. The diagnostic platform of claim 1, wherein the electrode layer comprises at least one working electrode.

10. The diagnostic platform of claim 9, wherein the at least one working electrode comprises a conductive film.

11. The diagnostic platform of claim 10, wherein the conductive film comprises gold, indium tin oxide (ITO), or a combination thereof.

12. The diagnostic platform of claim 1, wherein the sensing device comprises a thickness shear mode (TSM) transducer.

13. The diagnostic platform of claim 1, wherein sensing layer comprises an antigen or an antibody that is specific for the at least one biomarker.

14. The diagnostic platform of claim 13, wherein the antigen or antibody specific for the at least one biomarker is immobilized on the electrode layer.

15. The diagnostic platform of claim 14, wherein the at least one biomarker is immobilized by self-assembled monolayer (SAM).

16. The diagnostic platform of claim 1, wherein the disease or condition is caused by a coronavirus.

17. The diagnostic platform of claim 1, wherein the disease or condition comprises COVID-19, severe acute respiratory syndrome, a tissue inflammation or a combination thereof.

18. The diagnostic platform of claim 1, wherein the sensing layer comprises:
a severe acute respiratory syndrome coronavirus 2 (SARS-COV-2) structural protein or an antibody thereto immobilized on a surface of the at least one electrode layer; and
IgG, IgM, IgA, or a combination thereof, wherein the IgG, IgM, IgA, or a combination thereof is tethered to the SARS-COV-2 structural protein.

19. The diagnostic platform of claim 18, wherein the SARS-COV-2 structural protein or the antibody thereto is immobilized to the surface of the at least one electrode layer via a self-assembled monolayer (SAM).

20. The diagnostic platform of claim 19, wherein the SAM comprises a streptavidin-biotin bond, a thiol-bond, or a combination thereof.

21. The diagnostic platform of claim 19, wherein the SARS-COV-2 structural protein comprises an S Protein, an N protein, an M protein, or a combination thereof.

22. The diagnostic platform of claim 1, comprising at least one stabilizing solution that is configured to extend the shelf life of the diagnostic platform for up to at least 12 months.

23. The diagnostic platform of claim 1, wherein the sensing device is communicatively linked with the electronic reading platform via a USB connection.

24. The diagnostic platform of claim 1, wherein the computing device comprises a mobile computing device, the diagnostic platform further comprising:
an application running on a processor of the mobile computing device, wherein the electronic reading platform is communicatively linked to the mobile computing device; and
the electronic reading platform is configured to transmit sensor data to the mobile computing device.

25. The diagnostic platform of claim 1, wherein the electronic reading platform is communicatively linked to the mobile computing device through one or more wireless communications protocols.

26. The diagnostic platform of claim 25, wherein the mobile computing device comprises a portable digital assistant, a tablet, a smartphone, a laptop, or a combination thereof.

27. The diagnostic platform of claim 1, wherein the at least one electrode layer comprises
a thickness of about 230 nm to about 300 nm, and
a diameter of about 200 μm to about 350 μm;
and the piezoelectric material comprises a thickness of about 10 μm to about 3 mm.

28. A method of predicting the existence or progression of a disease or condition or level of immunity in a patient, the method comprising:
obtaining a fluid sample from the patient;
placing the fluid sample on the sensing device of any one of claims 1-27;
permitting the diagnostic platform to determine the presence, absence, or amount of the at least one biomarker for the disease or condition;
permitting the diagnostic platform to generate a sensor data report;
reviewing the sensor data report; and
predicting the presence or progression of the disease or condition.

29. The method of claim 28, wherein
the disease or condition is COVID-19, SARS, or a combination thereof;
the at least one biomarker is selected from an IgM antibody, an IgA antibody, an IgG antibody, or a combination thereof, wherein
the presence of IgM and IgA antibodies but not IgG antibodies indicates that the patient is in an intermediate stage of infection;
the presence of IgG antibodies but not IgM or IgA antibodies indicates that either the patient is in a late stage or an early stage of recurring infection or the patient is in a convalescent stage of infection;
the presence of IgM, IgA and IgG antibodies indicates that either the patient is in a late phase of the infection or the patient is in recovery stage of infection; and
the absence of IgG, IgA and IgM antibodies indicates that the patient does not have COVID-19, SARS, or a combination thereof.

30. The method of claim 28, wherein
the disease or condition is selected from COVID-19, SARS, or a combination thereof; and
the at least one biomarker comprises one or more monoclonal IgG, IgM, or IgA antibodies for an S protein, an N protein, an M protein, or any combination thereof, wherein the presence of any one or more of the biomarkers indicates the presence or progression of the disease or condition.
